(19) United States
(12) Patent Application Publication
(10) Pub. No.: US 2021/0130485 A1
(43) Pub. Date: May 6, 2021

(54) TRANSFERRIN RECEPTOR-BINDING POLYPEPTIDES AND USES THEREOF

(71) Applicant: Denali Therapeutics Inc., South San Francisco, CA (US)

(72) Inventors: Mark S. Dennis, South San Francisco, CA (US); Mihalis Kariolis, South San Francisco, CA (US); Wanda Kwan, South San Francisco, CA (US); Adam P. Silverman, South San Francisco, CA (US); Zachary K. Sweeney, South San Francisco, CA (US); Joy Yu Zuchero, South San Francisco, CA (US)

(73) Assignee: Denali Therapeutics Inc., South San Francisco, CA (US)

(21) Appl. No.: 16/921,506

(22) Filed: Jul. 6, 2020

Related U.S. Application Data
(63) Continuation of application No. PCT/US2019/012990, filed on Jan. 10, 2019.
(60) Provisional application No. 62/615,914, filed on Jan. 10, 2018.
(60) Provisional application No. 62/631,281, filed on Feb. 15, 2018.
(60) Provisional application No. 62/682,639, filed on Jun. 8, 2018.
(60) Provisional application No. 62/721,275, filed on Aug. 22, 2018.

(51) Int. Cl. C07K 16/28

(52) U.S. Cl. CPC C07K 16/2881; C07K 2317/526; C07K 2317/734; C07K 2317/55; C07K 2317/732; C07K 2317/71

(57) ABSTRACT

The present disclosure relates generally to Fc polypeptide dimers that contain a non-native transferrin receptor (TfR) binding site, do not substantially deplete reticulocytes in vivo, but retain binding to the Fcγ receptor (FcγR). The present disclosure also relates to an Fc polypeptide dimer that contains a non-native site that specifically binds TfR on one of the Fc polypeptides; a modification or modifications on the Fc polypeptide containing the TfR-binding site that reduces FcγR binding when bound to TfR, where the other Fc polypeptide does not contain a TfR-binding site but retains FcγR binding.

### CROSS-REFERENCE TO RELATED APPLICATIONS

The present application is a continuation of International Application No. PCT/US2019/012990, filed Jan. 10, 2019, which claims priority to U.S. Provisional Patent Application No. 62/615,914, filed on Jan. 10, 2018, U.S. Provisional Patent Application No. 62/631,281, filed on Feb. 15, 2018, U.S. Provisional Patent Application No. 62/682,639, filed on Jun. 8, 2018, and U.S. Provisional Patent Application No. 62/721,275, filed on Aug. 22, 2018, the disclosures of which are incorporated herein by reference in their entirety for all purposes.

### FIELD

The present disclosure relates to modified Fc polypeptide dimers that bind transferrin receptor (TfR), can induce at least one effector function activity (e.g., antibody-dependent cellular cytotoxicity (ADCC)), but do not result in substantial depletion of reticulocytes.

### BACKGROUND

TfR has been proposed as a target for receptor-mediated transcytosis of therapeutics across the blood-brain barrier (BBB). While TfR is expressed on the endothelial cells that form the BBB, TfR is also expressed on other cell types, including reticulocytes. Previous work has shown that anti-TfR antibodies can deplete reticulocytes from circulation.

Because reticulocyte depletion is mediated by effector function activity, this toxicity can be overcome by making modifications that reduce or eliminate effector function. This approach, however, precludes the use of therapeutics where effector function is desired or required.

Thus, a means for delivering effector function-positive therapeutics to the brain that do not cause reticulocyte depletion would be advantageous.

### SUMMARY

We have developed Fc polypeptides that have been modified to bind to TfR. These Fc polypeptides are capable of being actively transported into the brain by receptor-mediated transcytosis through binding to TfR at the BBB. Because Fc polypeptides are capable of inducing effector function activity, including ADCC, through binding to Fcγ receptors (FcγR) on immune cells and because TfR is expressed on reticulocytes, simultaneous binding by these polypeptides to reticulocytes and FcγR can lead to reticulocyte depletion. While effector function can be reduced or eliminated by introducing mutations into the Fc polypeptide, this is not desirable in some therapeutic applications.

The present disclosure is based on the development of modified Fc polypeptide dimers that bind TfR, cross the BBB, and retain effector function activity but do not cause substantial TfR-dependent toxicity, including depletion of reticulocytes. Such dimers can be engineered as described herein.

In one aspect, the disclosure features a modified Fc polypeptide dimer, or a dimeric fragment thereof, that: (a) comprises a TfR-binding site that specifically binds TfR; (b) is capable of binding an Fcγ receptor (FcγR); and (c) does not substantially deplete reticulocytes in vivo.

In one aspect, the disclosure features a modified Fc polypeptide dimer, or a dimeric fragment thereof, comprising: (a) a first Fc polypeptide that specifically binds TfR comprising (i) a TfR-binding site and (ii) one or more amino acid modifications that reduce FcγR binding, for example, when bound to TfR (e.g., but has limited or no reduction of FcγR binding when not bound to TfR); and (b) a second Fc polypeptide that does not contain a TfR-binding site or any modifications that reduce FcγR binding.

In some embodiments of this aspect, the TfR-binding site comprises a modified CH3 domain. In some embodiments, the modified CH3 domain is derived from a human IgG1, IgG2, IgG3, or IgG4 CH3 domain. In particular embodiments, the modified CH3 domain comprises five, six, seven, eight, or nine substitutions in a set of amino acid positions comprising 384, 386, 387, 388, 389, 390, 413, 416, and 421, according to EU numbering. In particular embodiments, the modified CH3 domain further comprises one, two, three, or four substitutions at positions comprising 380, 391, 392, and 415.

In some embodiments, the modified CH3 domain further comprises one, two, or three substitutions at positions comprising 414, 424, and 426.

In some embodiments, the modified Fc polypeptide dimer binds to the apical domain of TfR. In some embodiments, the modified Fc polypeptide dimer binds to TfR without inhibiting binding of transferrin to TfR. In particular embodiments, the modified Fc polypeptide dimer binds to an epitope that comprises amino acid 208 of TfR.

In some embodiments, the modified CH3 domain comprises Trp at position 388. In some embodiments, the modified CH3 domain comprises an aromatic amino acid at position 421. In particular embodiments, the aromatic amino acid at position 421 is Trp or Phe.

In some embodiments of this aspect, the modified CH3 domain comprises at least one position selected from the following: position 384 is Leu, Tyr, Met, or Val; position 386 is Leu, Thr, His, or Pro; position 387 is Val, Pro, or an acidic amino acid; position 388 is Trp; position 389 is Val, Ser, or Ala; position 413 is Glu, Ala, Ser, Leu, Thr, or Pro; position 416 is Thr or an acidic amino acid; and position 421 is Trp, Tyr, His, or Phe.

In some embodiments of this aspect, the modified CH3 domain comprises two, three, four, five, six, seven, or eight positions selected from the following: position 384 is Leu, Tyr, Met, or Val; position 386 is Leu, Thr, His, or Pro; position 387 is Val, Pro, or an acidic amino acid; position 388 is Trp; position 389 is Val, Ser, or Ala; position 413 is Glu, Ala, Ser, Leu, Thr, or Pro; position 416 is Thr or an acidic amino acid; and position 421 is Trp, Tyr, His, or Phe.

In some embodiments of this aspect, the modified CH3 domain comprises Leu or Met at position 384; Leu, His, or Pro at position 386; Val at position 387; Trp at position 388; Val or Ala at position 389; Pro at position 413; Thr at position 416; and/or Trp at position 421.

In some embodiments, the modified CH3 domain further comprises Ser, Thr, Gln, or Phe at position 391. In some embodiments, the modified CH3 domain further comprises Trp, Tyr, Leu, or Gln at position 380. In some embodiments, the modified CH3 domain further comprises Gln, Phe, or His at position 392. In some embodiments, the modified CH3 domain further comprises Trp at position 380 and/or Gln at position 392.

In some embodiments, the modified CH3 domain further comprises one, two, or three positions selected from the following: position 414 is Lys, Arg, Gly, or Pro; position 424 is Ser, Thr, Glu, or Lys; and position 426 is Ser, Trp, or Gly.

In some embodiments, the modified CH3 domain comprises Tyr at position 384, Thr at position 386, Glu or Val and position 387, Trp at position 388, Ser at position 389, Ser or Thr at position 413, Glu at position 416, and/or Phe at position 421. In some embodiments, the modified CH3 domain further comprises Trp, Tyr, Leu, or Gln at position 380. In some embodiments, the modified CH3 domain further comprises Glu at position 415. In some embodiments, the modified CH3 domain further comprises Trp at position 380 and/or Glu at position 415. In some embodiments, the modified CH3 domain comprises Asn at position 390.

In some embodiments, the modified CH3 domain comprises one or more of the following substitutions: Trp at position 380; Thr at position 386; Trp at position 388; Val at position 389; Ser or Thr at position 413; Glu at position 415; and/or Phe at position 421.

In some embodiments, the modified CH3 domain has at least 85% identity, at least 90% identity, or at least 95% identity to amino acids 111-217 of any one of SEQ ID NOS:4-29 and 64-127. In particular embodiments, the modified CH3 domain has at least 85% identity, at least 90% identity, or at least 95% identity to amino acids 111-217 of any one of SEQ ID NOS:66, 68, 94, 107-109, 119, and 268-270.

In some embodiments, the modified CH3 domain has at least 85% identity to amino acids 111-217 of SEQ ID NO:1 with the proviso that the percent identity does not include the set of positions 384, 386, 387, 388, 389, 390, 413, 416, and 421, according to EU numbering.

In some embodiments, the modified CH3 domain comprises amino acids 154-160 and/or 183-191 of any one of SEQ ID NOS:4-29 and 125-127.

In some embodiments, the modified CH3 domain comprises at least one position selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe. In some embodiments, the modified CH3 domain comprises 2, 3, 4, 5, 6, 7, 8, 9, 10, or 11 positions selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

In some embodiments, the modified CH3 domain comprises 11 positions as follows: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

In some embodiments, the modified CH3 domain has at least 85% identity, at least 90% identity, or at least 95% identity to amino acids 111-217 of any one of SEQ ID NOS:4-29 and 64-127. In particular embodiments, the modified CH3 domain has at least 85% identity, at least 90% identity, or at least 95% identity to amino acids 111-217 of any one of SEQ ID NOS:66, 68, 94, 107-109, 119, and 268-270.

In some embodiments, the residues at at least 5, 6, 7, 8, 9, 10, 11, 12, 13, 14, 15, or 16 of the positions corresponding to positions 380, 384, 386, 384, 388, 389, 390, 391, 392, 413, 414, 415, 416, 421, 424, and 426, according to EU numbering scheme, are not deleted or substituted.

In some embodiments, the modified CH3 domain comprises a sequence of any one of SEQ ID NOS:38-61 and 131-173.

In some embodiments of this aspect, the modified CH3 domain further comprises (i) a Trp at position 366 or (ii) a Ser at position 366, an Ala at position 368, and a Val at position 407, according to EU numbering scheme.

In some embodiments of this aspect, the corresponding unmodified CH3 domain is a human IgG1, IgG2, IgG3, or IgG4 CH3 domain.

In some embodiments of this aspect, the amino acid modifications that reduce FcγR binding, e.g., when bound to TfR, comprise Ala at position 234 and at position 235, according to EU numbering scheme. In some embodiments, the amino acid modifications that reduce FcγR binding, e.g., when bound to TfR, further comprise Gly at position 329, according to EU numbering scheme.

In some embodiments of this aspect, the first Fc polypeptide and/or the second Fc polypeptide comprises amino acid modifications that increase serum stability (e.g., serum half-life). In some embodiments, the amino acid modifications that increase serum stability (e.g., serum half-life) comprise Tyr at position 252, Thr at position 254, and Glu at position 256, according to EU numbering scheme. In some embodiments, the amino acid modifications that increase serum stability (e.g., serum half-life) comprise (i) a Leu at position 428 and a Ser at position 434, or (ii) a Ser or Ala at position 434, according to EU numbering scheme.

In some embodiments of this aspect, the modified Fc polypeptide dimer is further fused to a Fab. In some embodiments, the first Fc polypeptide and/or the second Fc polypeptide is further fused to a Fab.

In some embodiments, the first Fc polypeptide comprises a knob mutation T366W and the second Fc polypeptide comprises hole mutations T366S, L368A, and Y407V, according to EU numbering scheme. In some embodiments, the first Fc polypeptide comprises hole mutations T366S, L368A, and Y407V and the second Fc polypeptide comprises a knob mutation T366W, according to EU numbering scheme.

In another aspect, the disclosure features a modified Fc polypeptide dimer, comprising: (a) a first Fc polypeptide that specifically binds TfR comprising a TfR-binding site, amino acid modifications L234A and L235A, and a knob mutation T366W, according to EU numbering scheme, and (b) a second Fc polypeptide that comprises hole mutations T366S, L368A, and Y407V, according to EU numbering scheme, and does not contain a TfR-binding site or any modifications that reduce FcγR binding. In some embodiments, the first Fc polypeptide comprises the sequence of any one of SEQ ID NOS:178, 190, 202, 214, 226, 238, 238, 252, 286, 298, and 310. In some embodiments, the second Fc polypeptide comprises the sequence of SEQ ID NO:397.

In another aspect, the disclosure features a modified Fc polypeptide dimer, comprising: (a) a first Fc polypeptide that specifically binds TfR comprising a TfR-binding site, amino acid modifications L234A, L235A, and P329G, and a knob mutation T366W, according to EU numbering scheme, and (b) a second Fc polypeptide that comprises hole mutations T366S, L368A, and Y407V, according to EU numbering scheme, and does not contain a TfR-binding site or any modifications that reduce FcγR binding. In some embodiments, the first Fc polypeptide comprises the sequence of any one of SEQ ID NOS:179, 191, 203, 215, 227, 239, 275, 287, 299, and 311. In some embodiments, the second Fc polypeptide comprises the sequence of SEQ ID NO:397.

In another aspect, the disclosure features a modified Fc polypeptide dimer, comprising: (a) a first Fc polypeptide that specifically binds TfR comprising a TfR-binding site, amino acid modifications L234A and L235A, a knob mutation T366W, and amino acid modifications M252Y, S254T, and T256E, according to EU numbering scheme, and (b) a second Fc polypeptide that comprises hole mutations T366S, L368A, and Y407V, according to EU numbering scheme, and does not contain a TfR-binding site or any modifications that reduce FcγR binding. In some embodiments, the first Fc polypeptide comprises the sequence of any one of SEQ ID NOS:181, 193, 205, 217, 229, 241, 277, 289, 301, and 313. In some embodiments, the second Fc polypeptide comprises the sequence of SEQ ID NO:397.

In another aspect, the disclosure features a modified Fc polypeptide dimer, comprising: (a) a first Fc polypeptide that specifically binds TfR comprising a TfR-binding site, amino acid modifications L234A and L235A, a knob mutation T366W, and amino acid modification N434S with or without M428L, according to EU numbering scheme, and (b) a second Fc polypeptide that comprises hole mutations T366S, L368A, and Y407V, according to EU numbering scheme, and does not contain a TfR-binding site or any modifications that reduce FcγR binding. In some embodiments, the first Fc polypeptide comprises the sequence of any one of SEQ ID NOS:323, 330, 337, 344, 351, 358, 365, 372, 379, and 386. In some embodiments, the second Fc polypeptide comprises the sequence of SEQ ID NO:397.

In another aspect, the disclosure features a modified Fc polypeptide dimer, comprising: (a) a first Fc polypeptide that specifically binds TfR comprising a TfR-binding site, amino acid modifications L234A, L235A, and P329G, a knob mutation T366W, and amino acid modifications M252Y, S254T, and T256E, according to EU numbering scheme, and (b) a second Fc polypeptide that comprises hole mutations T366S, L368A, and Y407V, according to EU numbering scheme, and does not contain a TfR-binding site or any modifications that reduce FcγR binding. In some embodiments, the first Fc polypeptide comprises the sequence of any one of SEQ ID NOS:182, 194, 206, 218, 230, 242, 278, 290, 302, and 314. In some embodiments, the second Fc polypeptide comprises the sequence of SEQ ID NO:397.

In another aspect, the disclosure features a modified Fc polypeptide dimer, comprising: (a) a first Fc polypeptide that specifically binds TfR comprising a TfR-binding site, amino acid modifications L234A, L235A, and P329G, a knob mutation T366W, and amino acid modification N434S with or without M428L, according to EU numbering scheme, and (b) a second Fc polypeptide that comprises hole mutations T366S, L368A, and Y407V, according to EU numbering scheme, and does not contain a TfR-binding site or any modifications that reduce FcγR binding. In some embodiments, the first Fc polypeptide comprises the sequence of any one of SEQ ID NOS:324, 331, 338, 345, 352, 359, 366, 373, 380, and 387. In some embodiments, the second Fc polypeptide comprises the sequence of SEQ ID NO:397.

In another aspect, the disclosure features a modified Fc polypeptide dimer, comprising: (a) a first Fc polypeptide that specifically binds TfR comprising a TfR-binding site, amino acid modifications L234A and L235A, and a knob mutation T366W, according to EU numbering scheme, and (b) a second Fc polypeptide that comprises hole mutations T366S, L368A, and Y407V and amino acid modifications M252Y, S254T, and T256E, according to EU numbering scheme, and does not contain a TfR-binding site or any modifications that reduce FcγR binding. In some embodiments, the first Fc polypeptide comprises the sequence of any one of SEQ ID NOS:178, 190, 202, 214, 226, 238, 252, 286, 298, and 310. In some embodiments, the second Fc polypeptide comprises the sequence of SEQ ID NO:400.

In another aspect, the disclosure features a modified Fc polypeptide dimer, comprising: (a) a first Fc polypeptide that specifically binds TfR comprising a TfR-binding site, amino acid modifications L234A and L235A, and a knob mutation T366W, according to EU numbering scheme, and (b) a second Fc polypeptide that comprises hole mutations T366S, L368A, and Y407V and amino acid modification N434S with or without M428L, according to EU numbering scheme, and does not contain a TfR-binding site or any modifications that reduce FcγR binding. In some embodiments, the first Fc polypeptide comprises the sequence of any one of SEQ ID NOS:178, 190, 202, 214, 226, 238, 252, 286, 298, and 310. In some embodiments, the second Fc polypeptide comprises the sequence of SEQ ID NO:407.

In another aspect, the disclosure features a modified Fc polypeptide dimer, comprising: (a) a first Fc polypeptide that specifically binds TfR comprising a TfR-binding site, amino acid modifications L234A, L235A, and P329G, and a knob mutation T366W, according to EU numbering scheme, and (b) a second Fc polypeptide that comprises hole mutations T366S, L368A, and Y407V and amino acid modifications M252Y, S254T, and T256E, according to EU numbering scheme, and does not contain a TfR-binding site or any modifications that reduce FcγR binding. In some embodiments, the first Fc polypeptide comprises the sequence of any one of SEQ ID NOS:179, 191, 203, 215, 227, 239, 275, 287, 299, and 311. In some embodiments, the second Fc polypeptide comprises the sequence of SEQ ID NO:400.

In another aspect, the disclosure features a modified Fc polypeptide dimer, comprising: (a) a first Fc polypeptide that specifically binds TfR comprising a TfR-binding site, amino acid modifications L234A, L235A, and P329G, and a knob mutation T366W, according to EU numbering scheme, and (b) a second Fc polypeptide that comprises hole mutations T366S, L368A, and Y407V and amino acid modification N434S with or without M428L, according to EU numbering scheme, and does not contain a TfR-binding site or any modifications that reduce FcγR binding. In some embodiments, the first Fc polypeptide comprises the sequence of any one of SEQ ID NOS:179, 191, 203, 215, 227, 239, 275, 287, 299, and 311. In some embodiments, the second Fc polypeptide comprises the sequence of SEQ ID NO:407.

In another aspect, the disclosure features a modified Fc polypeptide dimer, comprising: (a) a first Fc polypeptide that specifically binds TfR comprising a TfR-binding site, amino acid modifications L234A and L235A, a knob mutation T366W, and amino acid modifications M252Y, S254T, and T256E, according to EU numbering scheme, and (b) a second Fc polypeptide that comprises hole mutations T366S, L368A, and Y407V and amino acid modifications M252Y, S254T, and T256E, according to EU numbering scheme, and does not contain a TfR-binding site or any modifications that reduce FcγR binding. In some embodiments, the first Fc polypeptide comprises the sequence of any one of SEQ ID NOS:181, 193, 205, 217, 229, 241, 277, 289, 301, and 313. In some embodiments, the second Fc polypeptide comprises the sequence of SEQ ID NO:400.

In another aspect, the disclosure features a modified Fc polypeptide dimer, comprising: (a) a first Fc polypeptide that specifically binds TfR comprising a TfR-binding site, amino acid modifications L234A and L235A, a knob mutation T366W, and amino acid modification N434S with or without M428L, according to EU numbering scheme, and (b) a second Fc polypeptide that comprises hole mutations T366S, L368A, and Y407V and amino acid modification N434S with or without M428L, according to EU numbering scheme, and does not contain a TfR-binding site or any modifications that reduce FcγR binding. In some embodiments, the first Fc polypeptide comprises the sequence of any one of SEQ ID NOS:323, 330, 337, 344, 351, 358, 365, 372, 379, and 386. In some embodiments, the second Fc polypeptide comprises the sequence of SEQ ID NO:407.

In another aspect, the disclosure features a modified Fc polypeptide dimer, comprising: (a) a first Fc polypeptide that specifically binds TfR comprising a TfR-binding site, amino acid modifications L234A, L235A, and P329G, a knob mutation T366W, and amino acid modifications M252Y, S254T, and T256E, according to EU numbering scheme, and (b) a second Fc polypeptide that comprises hole mutations T366S, L368A, and Y407V and amino acid modifications M252Y, S254T, and T256E, according to EU numbering scheme, and does not contain a TfR-binding site or any modifications that reduce FcγR binding. In some embodiments, the first Fc polypeptide comprises the sequence of any one of SEQ ID NOS:182, 194, 206, 218, 230, 242, 278, 290, 302, and 314. In some embodiments, the second Fc polypeptide comprises the sequence of SEQ ID NO:400.

In another aspect, the disclosure features a modified Fc polypeptide dimer, comprising: (a) a first Fc polypeptide that specifically binds TfR comprising a TfR-binding site, amino acid modifications L234A, L235A, and P329G, a knob mutation T366W, and amino acid modification N434S with or without M428L, according to EU numbering scheme, and (b) a second Fc polypeptide that comprises hole mutations T366S, L368A, and Y407V and amino acid modification N434S with or without M428L, according to EU numbering scheme, and does not contain a TfR-binding site or any modifications that reduce FcγR binding. In some embodiments, the first Fc polypeptide comprises the sequence of any one of SEQ ID NOS:324, 331, 338, 345, 352, 359, 366, 373, 380, and 387. In some embodiments, the second Fc polypeptide comprises the sequence of SEQ ID NO:407.

In another aspect, the disclosure features a modified Fc polypeptide dimer, comprising: (a) a first Fc polypeptide that specifically binds TfR comprising a TfR-binding site, amino acid modifications L234A and L235A, and hole mutations T366S, L368A, and Y407V, according to EU numbering scheme, and (b) a second Fc polypeptide that comprises a knob mutation T366W, according to EU numbering scheme, and does not contain a TfR-binding site or any modifications that reduce FcγR binding. In some embodiments, the first Fc polypeptide comprises the sequence of any one of SEQ ID NOS:184, 196, 208, 220, 232, 244, 280, 292, 304, and 316. In some embodiments, the second Fc polypeptide comprises the sequence of SEQ ID NO:391.

In another aspect, the disclosure features a modified Fc polypeptide dimer, comprising: (a) a first Fc polypeptide that specifically binds TfR comprising a TfR-binding site, amino acid modifications L234A, L235A, and P329G, and hole mutations T366S, L368A, and Y407V, according to EU numbering scheme, and (b) a second Fc polypeptide that comprises a knob mutation T366W, according to EU numbering scheme, and does not contain a TfR-binding site or any modifications that reduce FcγR binding. In some embodiments, the first Fc polypeptide comprises the sequence of any one of SEQ ID NOS:185, 197, 209, 221, 233, 245, 281, 293, 305, and 317. In some embodiments, the second Fc polypeptide comprises the sequence of SEQ ID NO:391.

In another aspect, the disclosure features a modified Fc polypeptide dimer, comprising: (a) a first Fc polypeptide that specifically binds TfR comprising a TfR-binding site, amino acid modifications L234A and L235A, hole mutations T366S, L368A, and Y407V, and amino acid modifications M252Y, S254T, and T256E, according to EU numbering scheme, and (b) a second Fc polypeptide that comprises a knob mutation T366W according to EU numbering scheme, and does not contain a TfR-binding site or any modifications that reduce FcγR binding. In some embodiments, the first Fc polypeptide comprises the sequence of any one of SEQ ID NOS:187, 199, 211, 223, 235, 247, 283, 295, 307, and 319. In some embodiments, the second Fc polypeptide comprises the sequence of SEQ ID NO:391.

In another aspect, the disclosure features a modified Fc polypeptide dimer, comprising: (a) a first Fc polypeptide that specifically binds TfR comprising a TfR-binding site, amino acid modifications L234A and L235A, hole mutations T366S, L368A, and Y407V, and amino acid modification N434S with or without M428L, according to EU numbering scheme, and (b) a second Fc polypeptide that comprises a knob mutation T366W according to EU numbering scheme, and does not contain a TfR-binding site or any modifications that reduce FcγR binding. In some embodiments, the first Fc polypeptide comprises the sequence of any one of SEQ ID NOS:326, 333, 340, 347, 354, 361, 368, 375, 382, and 389. In some embodiments, the second Fc polypeptide comprises the sequence of SEQ ID NO:391.

In another aspect, the disclosure features a modified Fc polypeptide dimer, comprising: (a) a first Fc polypeptide that specifically binds TfR comprising a TfR-binding site, amino acid modifications L234A, L235A, and P329G, hole mutations T366S, L368A, and Y407V, and amino acid modifications M252Y, S254T, and T256E, according to EU numbering scheme, and (b) a second Fc polypeptide that comprises a knob mutation T366W, according to EU numbering scheme, and does not contain a TfR-binding site or any modifications that reduce FcγR binding. In some embodiments, the first Fc polypeptide comprises the sequence of any one of SEQ ID NOS:188, 200, 212, 224, 236, 248, 284, 296, 308, and 320. In some embodiments, the second Fc polypeptide comprises the sequence of SEQ ID NO:391.

In another aspect, the disclosure features a modified Fc polypeptide dimer, comprising: (a) a first Fc polypeptide that specifically binds TfR comprising a TfR-binding site, amino acid modifications L234A, L235A, and P329G, hole mutations T366S, L368A, and Y407V, and amino acid modification N434S with or without M428L, according to EU numbering scheme, and (b) a second Fc polypeptide that comprises a knob mutation T366W, according to EU numbering scheme, and does not contain a TfR-binding site or any modifications that reduce FcγR binding. In some embodiments, the first Fc polypeptide comprises the sequence of any one of SEQ ID NOS:327, 334, 341, 348, 355, 362, 369, 376, 383, and 390. In some embodiments, the second Fc polypeptide comprises the sequence of SEQ ID NO:391.

In another aspect, the disclosure features a modified Fc polypeptide dimer, comprising: (a) a first Fc polypeptide that specifically binds TfR comprising a TfR-binding site, amino acid modifications L234A and L235A, and hole mutations T366S, L368A, and Y407V, according to EU numbering scheme, and (b) a second Fc polypeptide that comprises a knob mutation T366W and amino acid modifications M252Y, S254T, and T256E, according to EU numbering scheme, and does not contain a TfR-binding site or any modifications that reduce FcγR binding. In some embodiments, the first Fc polypeptide comprises the sequence of any one of SEQ ID NOS:184, 196, 208, 220, 232, 244, 280, 292, 304, and 316. In some embodiments, the second Fc polypeptide comprises the sequence of SEQ ID NO:394.

In another aspect, the disclosure features a modified Fc polypeptide dimer, comprising: (a) a first Fc polypeptide that specifically binds TfR comprising a TfR-binding site, amino acid modifications L234A and L235A, and hole mutations T366S, L368A, and Y407V, according to EU numbering scheme, and (b) a second Fc polypeptide that comprises a knob mutation T366W and amino acid modification N434S with or without M428L, according to EU numbering scheme, and does not contain a TfR-binding site or any modifications that reduce FcγR binding. In some embodiments, the first Fc polypeptide comprises the sequence of any one of SEQ ID NOS:184, 196, 208, 220, 232, 244, 280, 292, 304, and 316. In some embodiments, the second Fc polypeptide comprises the sequence of SEQ ID NO:404.

In another aspect, the disclosure features a modified Fc polypeptide dimer, comprising: (a) a first Fc polypeptide that specifically binds TfR comprising a TfR-binding site, amino acid modifications L234A, L235A, and P329G, and hole mutations T366S, L368A, and Y407V, according to EU numbering scheme, and (b) a second Fc polypeptide that comprises a knob mutation T366W and amino acid modifications M252Y, S254T, and T256E, according to EU numbering scheme, and does not contain a TfR-binding site or any modifications that reduce FcγR binding. In some embodiments, the first Fc polypeptide comprises the sequence of any one of SEQ ID NOS:185, 197, 209, 221, 233, 245, 281, 293, 305, and 317. In some embodiments, the second Fc polypeptide comprises the sequence of SEQ ID NO:394.

In another aspect, the disclosure features a modified Fc polypeptide dimer, comprising: (a) a first Fc polypeptide that specifically binds TfR comprising a TfR-binding site, amino acid modifications L234A, L235A, and P329G, and hole mutations T366S, L368A, and Y407V, according to EU numbering scheme, and (b) a second Fc polypeptide that comprises a knob mutation T366W and amino acid modification N434S with or without M428L, according to EU numbering scheme, and does not contain a TfR-binding site or any modifications that reduce FcγR binding. In some embodiments, the first Fc polypeptide comprises the sequence of any one of SEQ ID NOS:185, 197, 209, 221, 233, 245, 281, 293, 305, and 317. In some embodiments, the second Fc polypeptide comprises the sequence of SEQ ID NO:404.

In another aspect, the disclosure features a modified Fc polypeptide dimer, comprising: (a) a first Fc polypeptide that specifically binds TfR comprising a TfR-binding site, amino acid modifications L234A and L235A, hole mutations T366S, L368A, and Y407V, and amino acid modifications M252Y, S254T, and T256E, according to EU numbering scheme, and (b) a second Fc polypeptide that comprises a knob mutation T366W and amino acid modifications M252Y, S254T, and T256E, according to EU numbering scheme, and does not contain a TfR-binding site or any modifications that reduce FcγR binding. In some embodiments, the first Fc polypeptide comprises the sequence of any one of SEQ ID NOS:187, 199, 211, 223, 235, 247, 283, 295, 307, and 319. In some embodiments, the second Fc polypeptide comprises the sequence of SEQ ID NO:394.

In another aspect, the disclosure features a modified Fc polypeptide dimer, comprising: (a) a first Fc polypeptide that specifically binds TfR comprising a TfR-binding site, amino acid modifications L234A and L235A, hole mutations T366S, L368A, and Y407V, and amino acid modification N434S with or without M428L, according to EU numbering scheme, and (b) a second Fc polypeptide that comprises a knob mutation T366W and amino acid modification N434S with or without M428L, according to EU numbering scheme, and does not contain a TfR-binding site or any modifications that reduce FcγR binding. In some embodiments, the first Fc polypeptide comprises the sequence of any one of SEQ ID NOS:326, 333, 340, 347, 354, 361, 368, 375, 382, and 389. In some embodiments, the second Fc polypeptide comprises the sequence of SEQ ID NO:404.

In another aspect, the disclosure features a modified Fc polypeptide dimer, comprising: (a) a first Fc polypeptide that specifically binds TfR comprising a TfR-binding site, amino acid modifications L234A, L235A, and P329G, hole mutations T366S, L368A, and Y407V, and amino acid modifications M252Y, S254T, and T256E, according to EU numbering scheme, and (b) a second Fc polypeptide that comprises a knob mutation T366W and amino acid modifications M252Y, S254T, and T256E, according to EU numbering scheme, and does not contain a TfR-binding site or any modifications that reduce FcγR binding. In some embodiments, the first Fc polypeptide comprises the sequence of any one of SEQ ID NOS:188, 200, 212, 224, 236, 248, 284, 296, 308, and 320. In some embodiments, the second Fc polypeptide comprises the sequence of SEQ ID NO:394.

In another aspect, the disclosure features a modified Fc polypeptide dimer, comprising: (a) a first Fc polypeptide that specifically binds TfR comprising a TfR-binding site, amino acid modifications L234A, L235A, and P329G, hole mutations T366S, L368A, and Y407V, and amino acid modification N434S with or without M428L, according to EU numbering scheme, and (b) a second Fc polypeptide that comprises a knob mutation T366W and amino acid modification N434S with or without M428L, according to EU numbering scheme, and does not contain a TfR-binding site or any modifications that reduce FcγR binding. In some embodiments, the first Fc polypeptide comprises the sequence of any one of SEQ ID NOS:327, 334, 341, 348, 355, 362, 369, 376, 383, and 390. In some embodiments, the second Fc polypeptide comprises the sequence of SEQ ID NO:404.

In any aspects of the modified Fc polypeptide dimer described herein, the modified Fc polypeptide dimer does not substantially deplete reticulocytes (e.g., circulating reticulocytes). In some embodiments, an amount of reticulocytes depleted after administering the modified Fc polypeptide dimer is less than an amount of reticulocytes depleted after administering a control. In some embodiments, an amount of reticulocytes depleted after administering the modified Fc polypeptide dimer is less than 50%, 45%, 40%, 35%, 30%, 25%, 20%, 15%, 10%, 8%, 5%, 3%, 2%, or 1% of an amount of reticulocytes depleted after administering a control. In some embodiments, an amount of reticulocytes remaining after administering the modified Fc polypeptide dimer is more than an amount of reticulocytes remaining after administering a control. In some embodiments, an amount of reticulocytes remaining after administering the modified Fc polypeptide dimer is at least 1%, 5%, 10%, 15%, 20%, 25%, 30%, 35%, 40%, 45%, or 50% more than an amount of reticulocytes remaining after administering a control.

In any aspects of the modified Fc polypeptide dimer described herein, the modified Fc polypeptide dimer does not substantially deplete reticulocytes in bone marrow. In some embodiments, an amount of reticulocytes depleted in the bone marrow after administering the modified Fc polypeptide dimer is less than an amount of reticulocytes depleted in the bone marrow after administering a control. In some embodiments, an amount of reticulocytes depleted in the bone marrow after administering the modified Fc polypeptide dimer is less than 50%, 45%, 40%, 35%, 30%, 25%, 20%, 15%, 10%, 8%, 5%, 3%, 2%, or 1% of an amount of reticulocytes depleted in the bone marrow after administering a control. In some embodiments, an amount of reticulocytes remaining in the bone marrow after administering the modified Fc polypeptide dimer is more than an amount of reticulocytes remaining in the bone marrow after administering a control. In some embodiments, an amount of reticulocytes remaining in the bone marrow after administering the modified Fc polypeptide dimer is at least 1%, 5%, 10%, 15%, 20%, 25%, 30%, 35%, 40%, 45%, or 50% more than an amount of reticulocytes remaining in the bone marrow after administering a control.

In some embodiments, the control is a corresponding TfR-binding Fc dimer (i.e., having the same mutations that result in TfR binding as the modified Fc polypeptide dimer described above) with full effector function and/or contains no mutations that reduce FcγR binding.

In another aspect, the disclosure features an Fc polypeptide dimer-Fab fusion protein that is capable of being actively transported across the BBB, the Fc polypeptide dimer-Fab fusion protein comprising: (a) an antibody variable region that is capable of binding an antigen, or antigen-binding fragment thereof; and (b) a modified Fc polypeptide dimer comprising (i) a first Fc polypeptide that specifically binds TfR comprising a TfR-binding site and one or more amino acid modifications that reduce FcγR binding, for example, when bound to TfR (e.g., but has limited or no reduction of FcγR binding when not bound to TfR), and (ii) a second Fc polypeptide that does not contain a TfR-binding site or any modifications that reduce FcγR binding.

In some embodiments of this aspect, the amino acid modifications that reduce FcγR binding, e.g., when bound to TfR, comprise Ala at position 234 and at position 235, according to EU numbering scheme. In particular embodiments, the amino acid modifications that reduce FcγR binding, e.g., when bound to TfR, further comprise Gly at position 329, according to EU numbering scheme.

In some embodiments of this aspect, the first Fc polypeptide and/or the second Fc polypeptide comprises amino acid modifications that increase serum stability (e.g., serum half-life). In some embodiments, the amino acid modifications that increase serum stability (e.g., serum half-life) comprise Tyr at position 252, Thr at position 254, and Glu at position 256, according to EU numbering scheme. In some embodiments, the amino acid modifications that increase serum stability (e.g., serum half-life) comprise (i) a Leu at position 428 and a Ser at position 434, or (ii) a Ser or Ala at position 434, according to EU numbering scheme.

In some embodiments of this aspect, the antibody variable region sequence comprises a Fab domain. In some embodiments, the antibody variable region sequence comprises two antibody variable region heavy chains and two antibody variable region light chains, or respective fragments thereof.

In some embodiments, the Fc polypeptide or Fc polypeptide dimer is fucose deficient or afucosylated (e.g., as described herein).

In another aspect, the disclosure features a pharmaceutical composition comprising a modified Fc polypeptide dimer described herein and a pharmaceutically acceptable carrier.

In another aspect, the disclosure features a pharmaceutical composition comprising the Fc polypeptide dimer-Fab fusion protein described herein and a pharmaceutically acceptable carrier.

In another aspect, the disclosure features a method of transcytosis of a composition across an endothelium, comprising contacting the endothelium with a composition comprising a modified Fc polypeptide dimer described herein. In some embodiments, the endothelium is the BBB.

In another aspect, the disclosure features a method of transcytosis of a composition across an endothelium, comprising contacting the endothelium with a composition comprising an Fc polypeptide dimer-Fab fusion protein described herein. In some embodiments, the endothelium is the BBB.

### BRIEF DESCRIPTION OF THE DRAWINGS

FIGS. 1A and 1B are graphs showing that the TfR-binding Fc polypeptide dimer fused to an anti-BACE1 Fab, in which the TfR-binding Fc polypeptide dimer was modified to reduce FcγR binding with L234A and L235A (LALA) mutations (numbered with reference to EU numbering scheme) on both Fc polypeptides of the dimer, did not deplete reticulocytes either in blood (FIG. 1A) or bone marrow (FIG. 1B) in human TfR knock-in (TfR^{ms/hu} KI) mice.

FIGS. 2A-2D are graphs showing that the modified Fc polypeptide dimer fused to an anti-BACE1 Fab, in which the TfR-binding Fc polypeptide dimer has the LALA mutations in the cis configuration relative to the TfR-binding site (“cis-LALA”), did not deplete reticulocytes in blood (FIG. 2A: at 25 mg/kg; FIG. 2C: at 50 mg/kg) or bone marrow (FIG. 2B: at 25 mg/kg; FIG. 2D: at 50 mg/kg) in human TfR knock-in (TfR^{ms/hu} KI) mice, whereas the analogously modified Fc polypeptide dimer fused to an anti-BACE1 Fab, in which the Fc polypeptide dimer has the LALA mutations trans to the TfR-binding site, depleted reticulocytes in both blood and bone marrow.

FIGS. 3A and 3B are graphs showing that the cis-LALA modified Fc polypeptide dimer (FIG. 3A: CH3C.35.21; FIG. 3B: CH3C.35.23) fused to an anti-BACE1 Fab and a modified Fc polypeptide with LALA mutations on both Fc polypeptides fused to an anti-BACE1 Fab did not induce TfR-mediated ADCC, whereas the hIgG1 with the TfR-binding site but without LALA mutations induced ADCC on Ramos cells expressing endogenous TfR.

FIG. 4 is a graph showing that TfR-binding Fc polypeptide dimer (CH3C.35.21) had no effect on TfR-mediated complement dependent cytotoxicity (CDC) activity, while anti-TfR control antibody Ab204 induced CDC in CHO-hTfR cells.

FIG. 5 is a graph showing that the cis-LALA modified Fc polypeptide dimer fused to an anti-BACE1 Fab induced pSyk protein levels in primary human microglial cells, similar to that seen in the TfR-binding polypeptide with wild-type hIgG1, whereas the modified Fc polypeptide dimer with LALA mutations on both Fc polypeptides fused to an anti-BACE1 Fab did not induce pSyk.

FIGS. 6A and 6B are graphs showing that hIgG1 with a cis-LALA Fc polypeptide dimer and mCD20 Fab binding site elicited ADCC similar to that of the anti-mCD20 antibody and hIgG1 with a TfR-binding site and mCD20 Fab binding site (FIG. 6A). Similarly, hIgG1 with a cis-LALA Fc polypeptide dimer and hCD20 Fab binding site elicited Fab-mediated CDC to the same degree as anti-hCD20 and hIgG1 with a TfR-binding site and hCD20 Fab binding site (FIG. 6B).

FIGS. 7A and 7B are graphs showing that hIgG1 with a cis-LALA Fc polypeptide dimer and mCD20 Fab binding site elicited robust B cell depletion similar to the anti-mCD20 antibody and hIgG1 with a TfR-binding site and mCD20 Fab binding site (FIGS. 7A and 7B). These results demonstrate that the cis-LALA modified Fc polypeptide dimer retains its Fc function and has Fab-mediated effector function in vivo.

FIGS. 8A, 8B, and 8C are graphs showing that mice treated with anti-Aβ having a TfR-binding site (CH3C.35.23.4) with cis-LALA Fc polypeptide dimer elicited robust microglial recruitment towards Aβ plaques (FIG. 8A: % plaque area with microglial overlap;

FIG. 8B: the same data normalized to the control IgG) and reduced plaques sized at 30-125 μm² (FIG. 8C). These results suggest that anti-Aβ having a cis-LALA Fc polypeptide dimer retains robust effector function for microglial recruitment and the ability to reduce some Aβ plaques similar to anti-Aβ.

### DETAILED DESCRIPTION

### I. Introduction

Modified Fc polypeptide dimers that include a TfR-binding site are capable of crossing the BBB, as well as transporting therapeutics across the BBB. As described herein, these Fc polypeptide dimers, if not engineered to reduce effector function, can also deplete reticulocytes in vivo since reticulocytes also express TfR. Reticulocyte depletion can be avoided by introducing modifications that remove effector function in the Fc polypeptides of the Fc polypeptide dimer, i.e., modifications that remove or reduce Fey receptor (FcγR) binding (e.g., L234A and L235A (LALA) substitutions, numbered with reference to EU numbering scheme). This approach, however, is disadvantageous in cases where effector function is desired when the Fab portion of the molecule is bound to its target (e.g., a therapeutic target protein).

The present disclosure provides modified Fc polypeptide dimers that retain effector function but do not cause substantial depletion of reticulocytes. These modified Fc polypeptide dimers are also referred to as “effector function-positive, TfR-binding Fc polypeptide dimers” herein. In some embodiments, only one of the two Fc polypeptides (but not both Fc polypeptides) of the effector function-positive, TfR-binding Fc polypeptide dimer is modified to both reduce effector function and bind TfR. The other Fc polypeptide of the modified Fc polypeptide dimer does not contain a TfR-binding site or any modifications that reduce effector function, but it may contain mutations that enhance effector function. Effector function-positive, TfR-binding Fc polypeptide dimers that have only one of the two Fc polypeptides containing both the TfR-binding site and modifications that reduce FcγR binding when bound to TfR, while the other Fc polypeptide does not contain a TfR-binding site or any modifications that reduce FcγR binding, are referred to as having the cis configuration. As described herein, these modified Fc polypeptide dimers having the cis configuration were tested for their effect on reticulocytes. These experiments demonstrated that by introducing both the TfR-binding site and mutations that reduce FcγR binding when bound to TfR to only one of the two polypeptides forming the modified Fc polypeptide dimer, it was possible to reduce effector function upon TfR binding, leading to TfR binding without substantial depletion of reticulocytes.

As described in detail herein, modified Fc polypeptide dimers having different configurations were fused to Fabs directed against a therapeutic target (e.g., CD20) to determine whether effector function (e.g., ADCC and CDC) could be retained when the Fab bound its target but not TfR. As described in detail below, particular configurations of (a) modifications that reduce FcγR binding, for example, when bound to TfR and (b) modifications that result in TfR binding in the modified Fc polypeptide dimer can produce, when the Fc polypeptide dimer is fused to a Fab, Fc polypeptide dimer-Fab fusions that still retain effector function (e.g., ADCC or CDC), but do not deplete reticulocytes. This approach allows the use of TfR-mediated transport across the BBB while retaining effector function.

Thus, the present disclosure relates, in part, to modified Fc polypeptide dimers that have been engineered to bind TfR, have reduced effector function (e.g., ADCC or CDC) when bound to TfR, but still retain effector function (e.g., ADCC or CDC) when the Fc polypeptide dimer is fused to a therapeutic Fab and bound to the Fab's target antigen.

### II. Definitions

As used herein, the singular forms “a,” “an,” and “the” include plural referents unless the content clearly dictates otherwise. Thus, for example, reference to “a polypeptide” may include two or more such molecules, and the like.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region. In general, an Fc polypeptide does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

As used herein, the term “Fe polypeptide dimer” refers to a dimer of two Fc polypeptides. In some embodiments, an Fc polypeptide dimer is capable of binding an Fc receptor (e.g., FcγR). In an Fc polypeptide dimer, the two Fc polypeptides dimerize by the interaction between the two CH3 antibody constant domains. In some embodiments, the two Fc polypeptides may also dimerize via one or more disulfide bonds that form between the hinge domains of the two dimerizing Fc domain monomers. An Fc polypeptide dimer may be a wild-type Fc polypeptide dimer or a modified Fc polypeptide dimer. A wild-type Fc polypeptide dimer is formed by the dimerization of two wild-type Fc polypeptides. An Fc polypeptide dimer can be a heterodimer or a homodimer.

As used herein, the term “modified Fc polypeptide dimer” refers to an Fc polypeptide dimer that contains at least one modified Fc polypeptide. In some embodiments, a modified Fc polypeptide dimer contains two modified Fc polypeptides. A modified Fc polypeptide dimer may be a homodimer (i.e., contains two identical modified Fc polypeptides) or a heterodimer (i.e., contains two different Fc polypeptides in which at least one of the two Fc polypeptides is a modified Fc polypeptide).

A “transferrin receptor” or “TfR” as used herein refers to transferrin receptor protein 1. The human transferrin receptor 1 polypeptide sequence is set forth in SEQ ID NO:63. Transferrin receptor protein 1 sequences from other species are also known (e.g., chimpanzee, accession number XP_003310238.1; rhesus monkey, NP 001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full-length TfR protein includes a short N-terminal intracellular region, a transmembrane region, and a large extracellular domain. The extracellular domain is characterized by three domains: a protease-like domain, a helical domain, and an apical domain. The apical domain sequence of human transferrin receptor 1 is set forth in SEQ ID NO:31.

As used herein, the term “Fcγ receptor” or “FcγR” refers to one type of Fc receptors, which are classified based on the type of antibody that they recognized. FcγRs includes several members, FcγRT (CD64), FcγRIIA (CD32), FcγRIM (CD32), FcγRIIIA (CD16a), and FcγRIIIB (CD16b), which differ in their antibody affinities due to different molecular structures. FcγRs bind to the Fc portion of IgG class of antibodies and are crucial for inducing phagocytosis of opsonized microbes. FcγRs are found on the cell surface of cells in the immune system. FcγRs are responsible for eliciting immune system effector functions and are activated upon binding of the Fc portion of an antibody to the receptor. FcγRs mediate immune functions, e.g., binding to antibodies that are attached to infected cells or invading pathogens, stimulating phagocytic or cytotoxic cells to destroy microbes or infected cells by antibody-mediated phagocytosis or ADCC.

As used herein, the term “reduce FcγR binding” refers to a modified Fc polypeptide or a modified Fc polypeptide dimer that contains mutations in the CH3 domain of the modified Fc polypeptide, in which the mutations decrease the affinity of the modified Fc polypeptide to the FcγR by 0.01% to 90% (e.g., 0.1%, 0.5%, 1%, 5%, 10%, 15%, 20%, 25%, 30%, 35%, 40%, 45%, 50%, 55%, 60%, 65%, 70%, 75%, 80%, 85%, or 90%) compared the affinity of an Fc polypeptide that does not contain mutations to reduce FcγR binding (e.g., a wild-type Fc polypeptide dimer). FcγR binding may be measured using, e.g., Surface Plasmon Resonance (SPR) methods (e.g., a Biacore™ system). Alternatively, FcγR binding can be measured using a functional assay, for example, an ADCC assay such as one described herein (e.g., an in vivo or in vitro assay of cell killing). The reduction of FcγR binding may be measured when the modified Fc polypeptide or modified Fc polypeptide dimer is bound to TfR. In some embodiments, the modified Fc polypeptide or modified Fc polypeptide dimer may have reduced FcγR binding when bound to TfR, but limited (e.g., less than 25%, 20%, 15%, 10%, 8%, 5%, 3%, 2%, or 1% reduction) or no reduction when not bound to TfR.

As described further herein, a modified Fc polypeptide dimer may contain a first Fc polypeptide that has both a TfR-binding site and mutations that reduce FcγR binding when bound to TfR and a second Fc polypeptide that has neither a TfR-binding site nor mutations that reduce FcγR binding. Thus, upon TfR engagement, the resulting asymmetrical Fc polypeptide dimer having the first and second Fc polypeptides may have an overall reduced affinity for FcγR. By contrast, there may be limited (e.g., as described above) or no reduction in FcγR binding when not bound to TfR.

The term “FcRn” refers to the neonatal Fc receptor. Binding of Fc polypeptides to FcRn reduces clearance and increases serum half-life of the Fc polypeptide. The human FcRn protein is a heterodimer that is composed of a protein of about 50 kDa in size that is similar to a major histocompatibility (MHC) class I protein and a 02-microglobulin of about 15 kDa in size.

As used herein, an “FcRn binding site” refers to the region of an Fc polypeptide that binds to FcRn. In human IgG, the FcRn binding site, as numbered using the EU numbering scheme, includes L251, M252, 1253, 5254, R255, T256, M428, H433, N434, H435, and Y436. These positions correspond to positions 21 to 26, 198, and 203 to 206 of SEQ ID NO:1.

As used herein, a “native FcRn binding site” refers to a region of an Fc polypeptide that binds to FcRn and that has the same amino acid sequence as the region of a naturally occurring Fc polypeptide that binds to FcRn.

As used herein, the term “does not substantially deplete reticulocytes in vivo” means that the reduction in reticuloctyes (e.g., the reduction in bone marrow reticulocytes or circulating reticuloctyes) caused by an effector function-positive, TfR-binding Fc polypeptide dimer described herein, or an Fc polypeptide dimer-Fab fusion protein described herein that contains an effector function-positive, TfR-binding Fc polypeptide dimer, is less than (e.g., less than 80%, 75%, 70%, 65%, 60%, 55%, 50%, 45%, 40%, 35%, 30%, 25%, 20%, 15%, 10%, 8%, 5%, 3%, 2%, or 1% of) the reduction in reticulocytes (e.g., the reduction in bone marrow reticulocytes or circulating reticuloctyes) caused by a control, e.g., a corresponding TfR-binding Fc dimer with full effector function and/or contains no mutations that reduce FcγR binding, or an antibody containing a corresponding TfR-binding Fc dimer with full effector function and/or contains no mutations that reduce FcγR binding.

The term “does not substantially deplete reticulocytes in vivo” can also mean that the amount or percentage of the remaining reticuloctyes (e.g., the remaining reticuloctyes in the bone marrow or in circulation) after dosing an effector function-positive, TfR-binding Fc polypeptide dimer described herein, or an Fc polypeptide dimer-Fab fusion protein described herein that contains an effector function-positive, TfR-binding Fc polypeptide dimer, is more than (e.g., at least 1%, 5%, 10%, 15%, 20%, 25%, 30%, 35%, 40%, 45%, or 50% more than) the amount or percentage of the remaining reticulocytes (e.g., the remaining reticuloctyes in the bone marrow or in circulation) after dosing a control (e.g., a corresponding TfR-binding Fc dimer with full effector function and/or contains no mutations that reduce FcγR binding, or an antibody containing a corresponding TfR-binding Fc dimer with full effector function and/or contains no mutations that reduce FcγR binding).

The amount or percentage of reticulocyte depletion (e.g., reticulocyte depletion in the bone marrow or in circulation), or the amount or percentage of remaining reticulocytes (e.g., remaining reticulocytes in the bone marrow or in circulation), may be measured in human TfR knock-in (TfR^{ms/hu} KI) mice (e.g., human TfR apical domain knock-in mice (“hTfR_{apical} knock-in mice”)), which are engineered to replace the mouse TfR with human apical domain/mouse chimeric TfR protein or in a non-human primate, such as a cynomolgus monkey. The measurement may be made by dosing the modified Fc dimer or control, e.g., 25 to 50 mg/kg intravenously (e.g., to the TfR^{ms/hu} KI mice) and circulating reticulocytes may be measured at 24 h post-dose by cytochemical reactions using the Advia 120 Hematology System, as described herein. Bone marrow reticulocytes can be measured using FACS sorting to determine the population of Ter119⁺, hCD71^{hi}, and FSC^{low} population, as described herein.

The terms “CH3 domain” and “CH2 domain” as used herein refer to immunoglobulin constant region domain polypeptides. In the context of IgG antibodies, a CH3 domain polypeptide refers to the segment of amino acids from about position 341 to about position 447 as numbered according to the EU numbering scheme, and a CH2 domain polypeptide refers to the segment of amino acids from about position 231 to about position 340 as numbered according to the EU numbering scheme. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. In the context of IgG antibodies, an Fc region refers to the segment of amino acids from about position 231 to about position 447 as numbered according to the EU numbering scheme. As used herein, the term “Fc region” may also include at least a part of a hinge region of an antibody. An illustrative hinge region sequence is set forth in SEQ ID NO:62.

The term “variable region” refers to a domain in an antibody heavy chain or light chain that derived from a germline Variable (V) gene, Diversity (D) gene, or Joining (J) gene (and not derived from a Constant (Cμ and Cδ gene segment), and that gives an antibody its specificity for binding to an antigen. Typically, an antibody variable region comprises four conserved “framework” regions interspersed with three hypervariable “complementarity determining regions.”

The terms “wild-type,” “native,” and “naturally occurring” with respect to a CH3 or CH2 domain are used herein to refer to a domain that has a sequence that occurs in nature.

As used herein, the term “mutant” with respect to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, γ-carboxyglutamate and O-phosphoserine. “Amino acid analogs” refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region as measured using one a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a polypeptide “corresponds to” an amino acid in the region of SEQ ID NO:1, when the residue aligns with the amino acid in SEQ ID NO:1 when optimally aligned to SEQ ID NO:1. The polypeptide that is aligned to the reference sequence need not be the same length as the reference sequence.

As used herein, the term “specifically binds” or “selectively binds” to a target, e.g., TfR or FcγR, when referring to a polypeptide comprising a modified CH3 domain as described herein, refers to a binding reaction whereby the polypeptide binds to the target with greater affinity, greater avidity, and/or greater duration than it binds to a structurally different target. In typical embodiments, the polypeptide has at least 5-fold, 6-fold, 7-fold, 8-fold, 9-fold, 10-fold, 20-fold, 25-fold, 50-fold, 100-fold, 1,000-fold, 10,000-fold, or greater affinity for a specific target, e.g., TfR or FcγR, compared to an unrelated target when assayed under the same affinity assay conditions. The term “specific binding,” “specifically binds to,” or “is specific for” a particular target (e.g., e.g., TfR or FcγR), as used herein, can be exhibited, for example, by a molecule having an equilibrium dissociation constant K_{D} for the target to which it binds of, e.g., 10⁻⁴ M or smaller, e.g., 10⁻⁵M, 10−6 M, 10−7 M, 10⁻⁸ M, 10⁻⁹ M, 10⁻¹⁰ M, 10⁻¹¹ M, or 10⁻¹² M. In some embodiments, a modified CH3 domain polypeptide specifically binds to an epitope on a TfR that is conserved among species (e.g., structurally conserved among species), e.g., conserved between non-human primate and human species (e.g., structurally conserved between non-human primate and human species). In some embodiments, a polypeptide may bind exclusively to a human TfR.

The term “binding affinity” as used herein refers to the strength of the non-covalent interaction between two molecules, e.g., a single binding site on a polypeptide and a target, e.g., TfR, to which it binds. Thus, for example, the term may refer to 1:1 interactions between a polypeptide and its target, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). As used herein, “binding affinity” includes not only formal binding affinities, such as those reflecting 1:1 interactions between a polypeptide and its target, but also apparent affinities for which K_{D}'s are calculated that may reflect avid binding.

### III. TfR-Binding Fc Polypeptides

This section describes generation of modified Fc polypeptides that bind to TfR and are capable of being transported across the blood-brain barrier (BBB).

### CH3 TfR-Binding Polypeptides

In some embodiments, the modified Fc polypeptide contains a modified human Ig CH3 domain, such as an IgG CH3 domain. The CH3 domain can be of any IgG subtype, i.e., from IgG1, IgG2, IgG3, or IgG4. In the context of IgG antibodies, a CH3 domain refers to the segment of amino acids from about position 341 to about position 447 as numbered according to the EU numbering scheme. The positions in the CH3 domain for purposes of identifying the corresponding set of amino acid positions for TfR binding are determined with reference to EU numbering scheme, SEQ ID NO:3, or amino acids 111-217 of SEQ ID NO:1 unless otherwise specified. Substitutions are also determined with reference to EU numbering scheme or SEQ ID NO:1, i.e., an amino acid is considered to be a substitution relative to the amino acid at the corresponding position in EU numbering scheme or SEQ ID NO:1.

As indicated above, sets of residues of a CH3 domain that can be modified are numbered herein with reference to EU numbering scheme or SEQ ID NO:1. Any CH3 domain, e.g., an IgG1, IgG2, IgG3, or IgG4 CH3 domain, may have modifications, e.g., amino acid substitutions, in one or more sets of residues that correspond to residues at the noted positions in EU numbering scheme or SEQ ID NO:1. The positions of each of the IgG1, IgG2, IgG3, and IgG4 sequences that correspond to any given position of EU numbering scheme or SEQ ID NO:1 can be readily determined.

One of skill understands that CH3 domains of other immunoglobulin isotypes, e.g., IgM, IgA, IgE, IgD, etc. may be similarly modified by identifying the amino acids in those domains that correspond to the amino acid positions described herein. Modifications may also be made to corresponding domains from immunoglobulins from other species, e.g., non-human primates, monkey, mouse, rat, rabbit, dog, pig, chicken, and the like.

In one embodiment, a modified CH3 domain polypeptide that specifically binds TfR binds to the apical domain of the TfR at an epitope that comprises position 208 of the full length human TfR sequence (SEQ ID NO:63), which corresponds to position 11 of the human TfR apical domain sequence set forth in SEQ ID NO:31. SEQ ID NO:31 corresponds to amino acids 198-378 of the human TfR-1 uniprotein sequence P02786 (SEQ ID NO:63). In some embodiments, the modified CH3 domain polypeptide binds to the apical domain of the TfR at an epitope that comprises positions 158, 188, 199, 207, 208, 209, 210, 211, 212, 213, 214, 215, and/or 294 of the full length human TfR sequence (SEQ ID NO:63). The modified CH3 domain polypeptide may bind to the TfR without blocking or otherwise inhibiting binding of transferrin to the receptor. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7%, 6%, 5%, 4%, 3%, 2%, or 1%). Illustrative CH3 domain polypeptides that exhibit this binding specificity include polypeptides having amino acid substitutions at positions 380, 384, 386, 387, 388, 389, 390, 413, 415, 416, and 421, according to the EU numbering scheme.

CH3 TfR Binding Set: 384, 386, 387, 388, 389, 390, 413, 416, and 421

In some embodiments, a modified CH3 domain polypeptide comprises one, two, three, four, five, six, seven, eight, nine, ten, or eleven substitutions in a set of amino acid positions comprising 380, 384, 386, 387, 388, 389, 390, 413, 415, 416, and 421, according to the EU numbering scheme (set CH3C). Illustrative substitutions that may be introduced at these positions are shown in Table 3. Additional substitutions are shown in Table 4. In some embodiments, the amino acid at position 388 and/or 421 is an aromatic amino acid, e.g., Trp, Phe, or Tyr. In some embodiments, the amino acid at position 388 is Trp. In some embodiments, the amino acid at position 388 is Gly. In some embodiments, the aromatic amino acid at position 421 is Trp or Phe.

In certain embodiments, the modified CH3 domain polypeptide comprises one, two, three, four, five, six, seven, eight, nine, ten, or eleven positions selected from the following: Glu, Leu, Ser, Val, Trp, Tyr, or Gln at position 380; Leu, Tyr, Phe, Trp, Met, Pro, or Val at position 384; Leu, Thr, His, Pro, Asn, Val, or Phe at position 386; Val, Pro, Ile, or an acidic amino acid at position 387; Trp at position 388; an aliphatic amino acid, Gly, Ser, Thr, or Asn at position 389; Gly, His, Gln, Leu, Lys, Val, Phe, Ser, Ala, Asp, Glu, Asn, Arg, or Thr at position 390; an acidic amino acid, Ala, Ser, Leu, Thr, Pro, Ile, or His at position 413; Glu, Ser, Asp, Gly, Thr, Pro, Gln, or Arg at position 415; Thr, Arg, Asn, or an acidic amino acid at position 416; and/or an aromatic amino acid, His, or Lys at position 421.

In some embodiments, a modified CH3 domain polypeptide that specifically binds to TfR comprises at least one position having a substitution, according to EU numbering scheme, as follows: Leu, Tyr, Met, or Val at position 384; Leu, Thr, His, or Pro at position 386; Val, Pro, or an acidic amino acid at position 387; an aromatic amino acid, e.g., Trp or Gly (e.g., Trp) at position 388; Val, Ser, or Ala at position 389; an acidic amino acid, Ala, Ser, Leu, Thr, or Pro at position 413; Thr or an acidic amino acid at position 416; or Trp, Tyr, His, or Phe at position 421. In some embodiments, a modified CH3 domain polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set. Thus, for example, Ile may be present at position 384, 386, and/or position 413. In some embodiments, the acidic amino acid at position one, two, or each of positions 387, 413, and 416 is Glu. In other embodiments, the acidic amino acid at one, two or each of positions 387, 413, and 416 is Asp. In some embodiments, two, three, four, five, six, seven, or all eight of positions 384, 386, 387, 388, 389, 413, 416, and 421 have an amino acid substitution as specified in this paragraph.

In some embodiments, a CH3 domain polypeptide having modifications in set CH3C comprises a native Asn at position 390. In some embodiments, the modified CH3 domain polypeptide comprises Gly, His, Gln, Leu, Lys, Val, Phe, Ser, Ala, or Asp at position 390. In some embodiments, the modified CH3 domain polypeptide further comprises one, two, three, or four substitutions at positions comprising 380, 391, 392, and 415. In some embodiments, Trp, Tyr, Leu, or Gln may be present at position 380. In some embodiments, Ser, Thr, Gln, or Phe may be present at position 391. In some embodiments, Gln, Phe, or His may be present at position 392. In some embodiments, Glu may be present at position 415.

In certain embodiments, the modified CH3 domain polypeptide comprises two, three, four, five, six, seven, eight nine, or ten positions selected from the following: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421. In some embodiments, the modified CH3 domain polypeptide comprises all eleven positions as follows: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421.

In certain embodiments, the modified CH3 domain polypeptide comprises Leu or Met at position 384; Leu, His, or Pro at position 386; Val at position 387; Trp at position 388; Val or Ala at position 389; Pro at position 413; Thr at position 416; and/or Trp at position 421. In some embodiments, the modified CH3 domain polypeptide further comprises Ser, Thr, Gln, or Phe at position 391. In some embodiments, a modified CH3 domain polypeptide further comprises Trp, Tyr, Leu, or Gln at position 380 and/or Gln, Phe, or His at position 392. In some embodiments, Trp is present at position 380 and/or Gln is present at position 392. In some embodiments, a modified CH3 domain polypeptide does not have a Trp at position 380.

In other embodiments, a modified CH3 domain polypeptide comprises Tyr at position 384; Thr at position 386; Glu or Val and position 387; Trp at position 388; Ser at position 389; Ser or Thr at position 413; Glu at position 416; and/or Phe at position 421. In some embodiments, the modified CH3 domain polypeptide comprises a native Asn at position 390. In certain embodiments, the modified CH3 domain polypeptide further comprises Trp, Tyr, Leu, or Gln at position 380; and/or Glu at position 415. In some embodiments, the modified CH3 domain polypeptide further comprises Trp at position 380 and/or Glu at position 415.

In additional embodiments, the modified CH3 domain further comprises one, two, or three positions selected from the following: position 414 is Lys, Arg, Gly, or Pro; position 424 is Ser, Thr, Glu, or Lys; and position 426 is Ser, Trp, or Gly.

In some embodiments, the modified CH3 domain comprises one or more of the following substitutions: Trp at position 380; Thr at position 386; Trp at position 388; Val at position 389; Ser or Thr at position 413; Glu at position 415; and/or Phe at position 421.

In some embodiments, a modified CH3 domain polypeptide that specifically binds TfR has at least 70% identity, at least 75% identity, at least 80% identity, at least 85% identity, at least 90% identity, or at least 95% identity to amino acids 111-217 of any one of SEQ ID NOS:4-29, 64-127, and 268-274 (e.g., SEQ ID NOS:66, 68, 94, 107-109, 119, and 268-270). In some embodiments, such a modified CH3 domain polypeptide comprises amino acids 154-160 and/or 183-191 of any one of SEQ ID NOS:4-29, 64-127, and 268-274 (e.g., SEQ ID NOS:66, 68, 94, 107-109, 119, and 268-270). In some embodiments, such a modified CH3 domain polypeptide comprises amino acids 150-160 and/or 183-191 of any one of SEQ ID NOS:4-29, 64-127, and 268-274 (e.g., SEQ ID NOS:66, 68, 94, 107-109, 119, and 268-270). In some embodiments, a modified CH3 domain polypeptide comprises amino acids 150-160 and/or 183-196 of any one of SEQ ID NOS:4-29, 64-127, and 268-274 (e.g., SEQ ID NOS:66, 68, 94, 107-109, 119, and 268-270).

In some embodiments, a modified CH3 domain polypeptide has at least 70% identity, at least 75% identity, at least 80% identity, at least 85% identity, at least 90% identity, or at least 95% identity to amino acids 111-217 of SEQ ID NO:1, with the proviso that the percent identity does not include the set of positions 154, 156, 157, 158, 159, 160, 183, 186, and 191 of SEQ ID NO:1 (positions 384, 386, 387, 388, 389, 390, 413, 416, and 421, according to EU numbering scheme). In some embodiments, the modified CH3 domain polypeptide comprises amino acids 154-160 and/or amino acids 183-191 as set forth in any one of SEQ ID NOS:4-29, 64-127, and 268-274 (e.g., SEQ ID NOS:66, 68, 94, 107-109, 119, and 268-270).

In some embodiments, a modified CH3 domain polypeptide has at least 70% identity, at least 75% identity, at least 80% identity, at least 85% identity, at least 90% identity, or at least 95% identity to any one of SEQ ID NOS:4-29, 64-127, and 268-274 (e.g., SEQ ID NOS:66, 68, 94, 107-109, 119, and 268-270), with the proviso that at least five, six, seven, eight, nine, ten, eleven, twelve, thirteen, fourteen, fifteen or sixteen of the positions that correspond to positions 150, 154, 156, 157, 158, 159, 160, 161, 162, 183, 184, 185, 186, 191, 194, and 196 of any one of SEQ ID NOS:4-29, 64-127, and 268-274 (e.g., SEQ ID NOS:66, 68, 94, 107-109, 119, and 268-270) (positions 380, 384, 386, 384, 388, 389, 390, 391, 392, 413, 414, 415, 416, 421, 424, and 426, according to EU numbering scheme) are not deleted or substituted.

In some embodiments, the modified CH3 domain polypeptide has at least 75% identity, at least 80% identity, at least 85% identity, at least 90% identity, or at least 95% identity to any one of SEQ ID NOS:4-29, 64-127, and 268-274 (e.g., SEQ ID NOS:66, 68, 94, 107-109, 119, and 268-270) and also comprises at least five, six, seven, eight, nine, ten, eleven, twelve, thirteen, fourteen, fifteen or sixteen of the positions as follows: Trp, Tyr, Leu, Gln, or Glu at position 380; Leu, Tyr, Met, or Val at position 384; Leu, Thr, His, or Pro at position 386; Val, Pro, or an acidic amino acid at position 387; an aromatic amino acid, e.g., Trp, at position 388; Val, Ser, or Ala at position 389; Ser or Asn at position 390; Ser, Thr, Gln, or Phe at position 391; Gln, Phe, or His at position 392; an acidic amino acid, Ala, Ser, Leu, Thr, or Pro at position 413; Lys, Arg, Gly or Pro at position 414; Glu or Ser at position 415; Thr or an acidic amino acid at position 416; Trp, Tyr, His or Phe at position 421; Ser, Thr, Glu or Lys at position 424; and Ser, Trp, or Gly at position 426.

In some embodiments, a TfR-binding polypeptide comprises the amino acid sequence of any one of SEQ ID NOS:38-52. In other embodiments, a TfR-binding polypeptide comprises the amino acid sequence of any one of SEQ ID NOS:38-52, but in which one or two amino acids are substituted. In some embodiments, the polypeptide comprises the amino acid sequence of any one of SEQ ID NOS:38-52, but in which three amino acids are substituted.

In some embodiments, a TfR-binding polypeptide comprises the amino acid sequence of any one of SEQ ID NOS:53-61. In other embodiments, a TfR-binding polypeptide comprises the amino acid sequence of any one of SEQ ID NOS:53-61, but in which one or two amino acids are substituted. In some embodiments, the polypeptide comprises the amino acid sequence of any one of SEQ ID NOS:53-61, but in which three or four amino acids are substituted.

In some embodiments, a TfR-binding polypeptide comprises the amino acid sequence of any one of SEQ ID NOS:131-167. In other embodiments, a TfR-binding polypeptide comprises the amino acid sequence of any one of SEQ ID NOS:131-167, but in which one or two amino acids are substituted. In some embodiments, the polypeptide comprises the amino acid sequence of any one of SEQ ID NOS:131-167, but in which three amino acids are substituted.

In some embodiments, a TfR-binding polypeptide comprises the amino acid sequence of any one of SEQ ID NOS:58, 60, and 168-173. In other embodiments, a TfR-binding polypeptide comprises the amino acid sequence of any one of SEQ ID NOS:58, 60, and 168-173, but in which one or two amino acids are substituted. In some embodiments, the polypeptide comprises the amino acid sequence of any one of SEQ ID NOS:58, 60, and 168-173, but in which three or four amino acids are substituted.

In additional embodiments, a TfR-binding polypeptide comprises amino acids 157-194, amino acids 153-194, or amino acids 153-199, of any one of SEQ ID NOS:4-29, 64-127, and 268-274 (e.g., SEQ ID NOS:66, 68, 94, 107-109, 119, and 268-270). In further embodiments, the polypeptide comprises an amino acid sequence having at least 75% identity, at least 80% identity, at least 85% identity, at least 90% identity, or at least 95% identity to amino acids 157-194 of any one of SEQ ID NOS:4-29, 64-127, and 268-274 (e.g., SEQ ID NOS:66, 68, 94, 107-109, 119, and 268-270), or to amino acids 153-194, or to amino acids 153-199, of any one of SEQ ID NOS:4-29, 64-127, and 268-274 (e.g., SEQ ID NOS:66, 68, 94, 107-109, 119, and 268-270).

In some embodiments, the polypeptide comprises any one of SEQ ID NOS:4-29, 64-127, and 268-274 (e.g., SEQ ID NOS:66, 68, 94, 107-109, 119, and 268-270). In further embodiments, the polypeptide may have at least 75% identity, at least 80% identity, at least 85% identity, at least 90% identity, or at least 95% identity to any one of SEQ ID NOS:4-29, 64-127, and 268-274 (e.g., SEQ ID NOS:66, 68, 94, 107-109, 119, and 268-270).

### FcRn Binding Sites

A polypeptide described herein that can be transported across the BBB additionally may comprise an FcRn binding site. In some embodiments, the FcRn binding site is within the modified Fc polypeptide or a fragment thereof.

In some embodiments, the FcRn binding site comprises a native FcRn binding site. In some embodiments, the FcRn binding site does not comprise amino acid changes relative to the amino acid sequence of a native FcRn binding site. In some embodiments, the native FcRn binding site is an IgG binding site, e.g., a human IgG binding site. In some embodiments, the FcRn binding site comprises a modification that alters FcRn binding.

In some embodiments, an FcRn binding site has one or more amino acid residues that are mutated, e.g., substituted, wherein the mutation(s) increase serum half-life or do not substantially reduce serum half-life (i.e., reduce serum half-life by no more than 25% compared to a counterpart protein having the wild-type residues at the mutated positions when assayed under the same conditions). In some embodiments, an FcRn binding site has one or more amino acid residues that are substituted at positions 21 to 26, 198, and 203 to 206, wherein the positions are determined with reference to SEQ ID NO:1.

In some embodiments, the FcRn binding site comprises one or more mutations, relative to a native human IgG sequence, that extend serum half-life of the modified polypeptide. In some embodiments, a mutation, e.g., a substitution, is introduced at one or more of positions 14-27, 49-54, 77-87, 153-160, and 198-205 as determined with reference to SEQ ID NO:1 (which positions correspond to positions 244-257, 279-284, 307-317, 383-390, and 428-435 using EU numbering). In some embodiments, one or more mutations are introduced at positions 21, 22, 24, 25, 26, 77, 78, 79, 81, 82, 84, 155, 156, 157, 159, 198, 203, 204, or 206 as determined with reference to SEQ ID NO:1 (which positions correspond to positions 251, 252, 254, 255, 256, 307, 308, 309, 311, 312, 314, 385, 386, 387, 389, 428, 433, 434, or 436 using EU numbering). In some embodiments, mutations are introduced into one, two, or three of positions 22, 24, and 25 as determined with reference to SEQ ID NO:1 (which correspond to positions 252, 254, and 256 using EU numbering). In some embodiments, the mutations are M22Y, S24T, and T26E as numbered with reference to SEQ ID NO:1. In some embodiments, a modified Fc polypeptide described herein further comprises mutations M22Y, S24T, and T26E. In some embodiments, mutations are introduced into one or two of positions 198 and 204 as determined with reference to SEQ ID NO:1 (which correspond to positions 428 and 434 using EU numbering). In some embodiments, the mutations are M198L and N204S as numbered with reference to SEQ ID NO:1. In some embodiments, a modified Fc polypeptide described herein further comprises mutation N204S with or without M198L. In some embodiments, a modified Fc polypeptide comprises a substitution at one, two or all three of positions T307, E380, and N434 according to EU numbering (which correspond to T77, E150, and N204 as numbered with reference to SEQ ID NO:1). In some embodiments, the mutations are T307Q and N434A (SEQ ID NO:1, T77Q and N204A). In some embodiments, a modified Fc polypeptide comprises mutations T307A, E380A, and N434A (SEQ ID NO:1, T77A, E150A, and N204A). In some embodiments, a modified Fc polypeptide comprises substitutions at positions T250 and M428 (which correspond to T20 and M198 as numbered with reference to SEQ ID NO:1). In some embodiments, the Fc polypeptide comprises mutations T250Q and/or M428L (SEQ ID NO:1, T20Q and M198L). In some embodiments, a modified Fc polypeptide comprises substitutions at positions M428 and N434 (which correspond to M198 and N204 as numbered with reference to SEQ ID NO:1). In some embodiments, a modified Fc polypeptide comprises substitutions M428L and N434S (which correspond to M198L and N204S as numbered with reference to SEQ ID NO:1). In some embodiments, a modified Fc polypeptide comprises an N434S or N434A substitution (which corresponds to N204S or N204A as numbered with reference to SEQ ID NO:1).

### IV. Mutations that Reduce Effector Function or FcγR Binding

An Fc polypeptide as provided herein that is modified to bind TfR and initiate transport across the BBB may also comprise additional mutations to reduce effector function. As described herein, by introducing both the TfR-binding site and mutations that reduce FcγR binding to the same Fc polypeptide of the Fc polypeptide dimer, it was possible to reduce effector function upon TfR binding, leading to TfR binding without substantial depletion of reticulocytes, but still retain effector function (e.g., ADCC or CDC) when the Fc polypeptide dimer is fused to a therapeutic Fab and bound to the Fab's target antigen.

In some embodiments, an Fc polypeptide comprising a modified CH3 domain has an effector function, i.e., the ability to induce certain biological functions upon binding to an Fc receptor expressed on an effector cell that mediates the effector function. Effector cells include, but are not limited to, monocytes, macrophages, neutrophils, dendritic cells, eosinophils, mast cells, platelets, B cells, large granular lymphocytes, Langerhans' cells, natural killer (NK) cells, and cytotoxic T cells.

Examples of effector functions include, but are not limited to, C1q binding and CDC, Fc receptor binding, ADCC, antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, an Fc polypeptide having an TfR-binding site as described herein may include additional modifications that reduce effector function, i.e., reduce effector function upon TfR binding. Having reduced effector function upon TfR binding of the Fc polypeptide dimer is desirable because it leads to reduced reticulocyte depletion since reticulocytes also have TfR on the cell surface. As described in detail herein, Fc polypeptide dimers having the cis configuration, i.e., Fc polypeptide dimers having both the TfR-binding site and mutations that reduce effector function on the same Fc polypeptide of the Fc polypeptide dimer, exhibit TfR binding without substantial depletion of reticulocytes, but still retain effector function (e.g., ADCC or CDC) when the Fc polypeptide dimer is fused to a therapeutic Fab and bound to the Fab's target antigen. Having effector function when the Fc polypeptide dimer is fused to a therapeutic Fab that is bound to the Fab's target antigen is desirable in, e.g., cancer therapeutics (e.g., brain cancer therapeutics).

Illustrative Fc polypeptide mutations that modulate an effector function include, but are not limited to, substitutions in a CH2 domain, e.g., at positions corresponding to positions 4 and 5 of SEQ ID NO:1 (positions 234 and 235 according to EU numbering scheme). In some embodiments, the substitutions in a modified CH2 domain comprise Ala at positions 4 and 5 of SEQ ID NO:1. In some embodiments, the substitutions in a modified CH2 domain comprise Ala at positions 4 and 5 and Gly at position 99 of SEQ ID NO:1.

Additional Fc polypeptide mutations that modulate an effector function include, but are not limited to, one or more substitutions at positions 238, 265, 269, 270, 297, 327 and 329 (EU numbering scheme, which correspond to positions 8, 35, 39, 40, 67, 97, and 99 as numbered with reference to SEQ ID NO:1). Illustrative substitutions (as numbered with EU numbering scheme), include the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, and P331 S. Multiple substitutions may also be present, e.g., L234A and L235A of a human IgG1 Fc region; L234A, L235A, and P329G of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region. In some embodiments, an Fc polypeptide may have one or more amino acid substitutions that modulate ADCC, e.g., substitutions at positions 298, 333, and/or 334 of the Fc region, according to the EU numbering scheme.

In some embodiments, a polypeptide as described herein may have one or more amino acid substitutions that increase or decrease ADCC or may have mutations that alter C1q binding and/or CDC.

In particular embodiments, an Fc polypeptide having a TfR-binding site may be modified to reduce effector function, i.e., reduce FcγR binding. In some embodiments, an Fc polypeptide having a TfR-binding site may include mutations L234A and L235A (EU numbering scheme, which correspond to positions 4 and 5 as numbered with reference to SEQ ID NO:1). In other embodiments, an Fc polypeptide having a TfR-binding site may include mutations L234A, L235A, and P329G (EU numbering scheme, which correspond to positions 4, 5, and 99 as numbered with reference to SEQ ID NO:1).

### V. Effector Function-Positive, TfR-Binding Fc Polypeptide Dimers

In certain aspects, the present disclosure provides effector function-positive, TfR-binding Fc polypeptide dimers that are modified to bind to TfR and to have reduced FcγR binding when bound to TfR, but have limited or no reduction of FcγR binding when not bound to TfR. These modified Fc polypeptide dimers may be fused to therapeutic Fabs to transport them across the BBB. These modified Fc polypeptide dimers are demonstrated to have reduced effector function upon TfR binding. When the modified Fc polypeptide dimer is fused to a Fab, the Fc polypeptide dimer retains effector function when the Fab is bound to its target (e.g., a target on a cancer cell). In this manner, the effector function-positive, TfR-binding Fc polypeptide dimers described herein are able to transport the Fab across the BBB without substantial depletion of reticulocytes (which also contain TfR on the cell surface), and also serve its therapeutic purpose by exhibiting effector function that can target extracellular aggregates (e.g., plaque) or certain diseased cells (e.g., cancer cells) in the brain to destruction when the Fab is bound to its target.

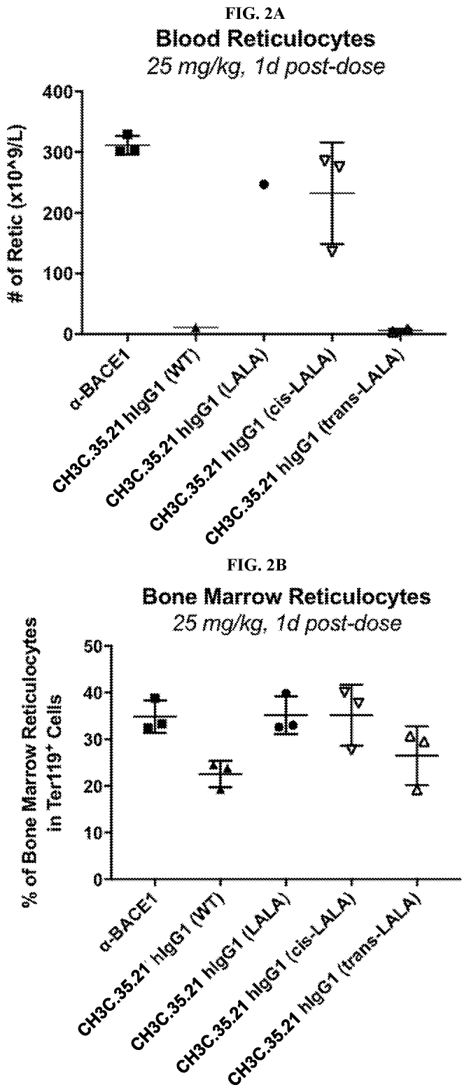

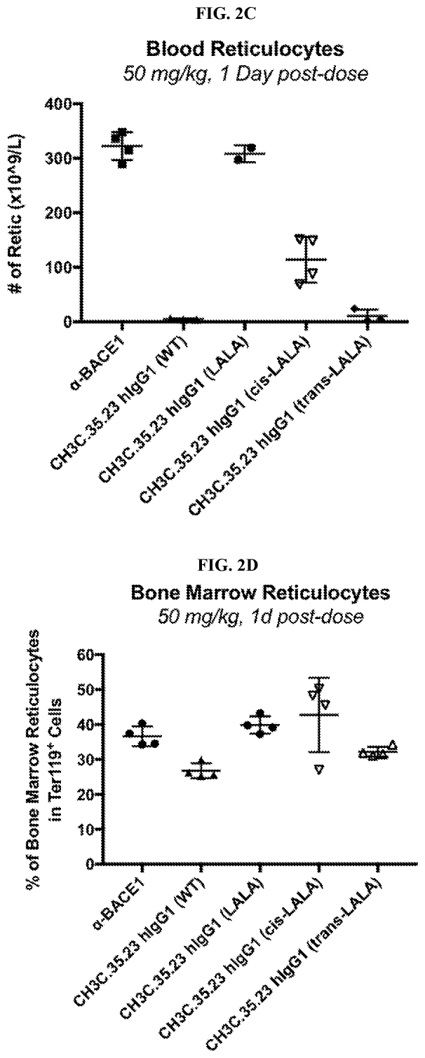

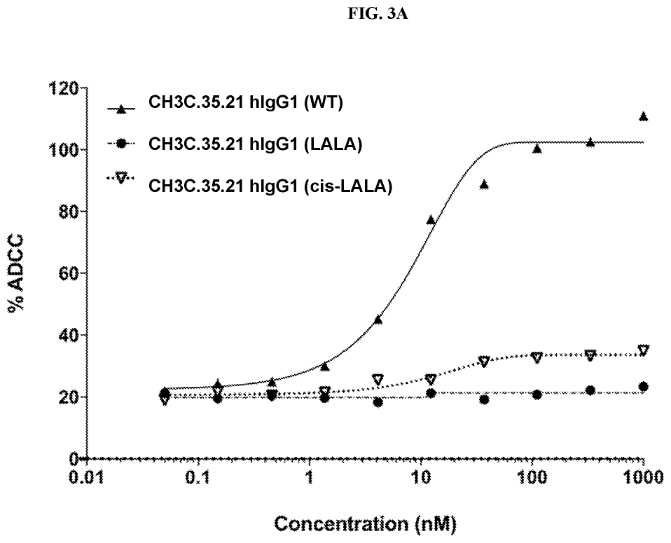

The effector function-positive, TfR-binding Fc polypeptide dimers described herein have a cis configuration, which means that only one (not both) of the Fc polypeptides in the Fc polypeptide dimer is modified to have a TfR-binding site and modifications that reduce FcγR binding when bound to TfR. The other Fc polypeptide in the Fc polypeptide dimer does not contain either a TfR-binding site or modifications that substantially reduce FcγR binding. A trans configuration of the modified Fc polypeptide dimers refers to an Fc polypeptide dimer in which one of the two Fc polypeptides contains a TfR-binding site, while the other Fc polypeptide contains modifications that reduce FcγR binding, e.g., when bound to TfR. As demonstrated herein, modified Fc polypeptide dimers having the cis configuration, but not the trans configuration, are able to reduce reticulocyte depletion in the blood and bone marrow (see, e.g., FIGS. 2A-2D).

In one embodiment, an effector function-positive, TfR-binding Fc polypeptide dimer comprises: (a) a first Fc polypeptide comprising a TfR-binding site that specifically binds TfR, and amino acid modifications L234A and L235A, according to EU numbering scheme, and (b) a second Fc polypeptide that does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, and amino acid modification N434S with or without M428L, according to EU numbering scheme, and (b) a second Fc polypeptide that does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, and amino acid modification N434S with or without M428L, according to EU numbering scheme, and (b) a second Fc polypeptide that comprises amino acid modification N434S with or without M428L and does not contain a TfR-binding site or any modifications that reduce FcγR binding.

In one embodiment, an effector function-positive, TfR-binding Fc polypeptide dimer comprises: (a) a first Fc polypeptide comprising a TfR-binding site that specifically binds TfR, amino acid modifications L234A and L235A, and a knob mutation T366W, according to EU numbering scheme, and (b) a second Fc polypeptide that comprises hole mutations T366S, L368A, and Y407V, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, and a knob mutation T366W, according to EU numbering scheme, and (b) a second Fc polypeptide that comprises hole mutations T366S, L368A, and Y407V, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, a knob mutation T366W, and amino acid modifications M252Y, S254T, and T256E, according to EU numbering scheme, and (b) a second Fc polypeptide that comprises hole mutations T366S, L368A, and Y407V, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, a knob mutation T366W, and amino acid modification N434S with or without M428L, according to EU numbering scheme, and (b) a second Fc polypeptide that comprises hole mutations T366S, L368A, and Y407V, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, a knob mutation T366W, and amino acid modifications M252Y, S254T, and T256E, according to EU numbering scheme, and (b) a second Fc polypeptide that comprises hole mutations T366S, L368A, and Y407V, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, a knob mutation T366W, and amino acid modification N434S with or without M428L, according to EU numbering scheme, and (b) a second Fc polypeptide that comprises hole mutations T366S, L368A, and Y407V, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, and a knob mutation T366W, according to EU numbering scheme, and (b) a second Fc polypeptide that comprises hole mutations T366S, L368A, and Y407V and amino acid modifications M252Y, S254T, and T256E, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, and a knob mutation T366W, according to EU numbering scheme, and (b) a second Fc polypeptide that comprises hole mutations T366S, L368A, and Y407V and amino acid modification N434S with or without M428L, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, and a knob mutation T366W, according to EU numbering scheme, and (b) a second Fc polypeptide that comprises hole mutations T366S, L368A, and Y407V and amino acid modifications M252Y, S254T, and T256E, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, and a knob mutation T366W, according to EU numbering scheme, and (b) a second Fc polypeptide that comprises hole mutations T366S, L368A, and Y407V and amino acid modification N434S with or without M428L, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, a knob mutation T366W, and amino acid modifications M252Y, S254T, and T256E, according to EU numbering scheme, and (b) a second Fc polypeptide that comprises hole mutations T366S, L368A, and Y407V and amino acid modifications M252Y, S254T, and T256E, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, a knob mutation T366W, and amino acid modification N434S with or without M428L, according to EU numbering scheme, and (b) a second Fc polypeptide that comprises hole mutations T366S, L368A, and Y407V and amino acid modification N434S with or without M428L, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, a knob mutation T366W, and amino acid modifications M252Y, S254T, and T256E, according to EU numbering scheme, and (b) a second Fc polypeptide that comprises hole mutations T366S, L368A, and Y407V and amino acid modifications M252Y, S254T, and T256E, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, a knob mutation T366W, and amino acid modification N434S with or without M428L, according to EU numbering scheme, and (b) a second Fc polypeptide that comprises hole mutations T366S, L368A, and Y407V and amino acid modification N434S with or without M428L, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, and hole mutations T366S, L368A, and Y407V, according to EU numbering scheme, and (b) a second Fc polypeptide that comprises a knob mutation T366W, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, and hole mutations T366S, L368A, and Y407V, according to EU numbering scheme, and (b) a second Fc polypeptide that comprises a knob mutation T366W, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, hole mutations T366S, L368A, and Y407V, and amino acid modifications M252Y, S254T, and T256E, according to EU numbering scheme, and (b) a second Fc polypeptide that comprises a knob mutation T366W according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, hole mutations T366S, L368A, and Y407V, and amino acid modification N434S with or without M428L, according to EU numbering scheme, and (b) a second Fc polypeptide that comprises a knob mutation T366W according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, hole mutations T366S, L368A, and Y407V, and amino acid modifications M252Y, S254T, and T256E, according to EU numbering scheme, and (b) a second Fc polypeptide that comprises a knob mutation T366W, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, hole mutations T366S, L368A, and Y407V, and amino acid modification N434S with or without M428L, according to EU numbering scheme, and (b) a second Fc polypeptide that comprises a knob mutation T366W, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, and hole mutations T366S, L368A, and Y407V, according to EU numbering scheme, and (b) a second Fc polypeptide that comprises a knob mutation T366W and amino acid modifications M252Y, S254T, and T256E, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, and hole mutations T366S, L368A, and Y407V, according to EU numbering scheme, and (b) a second Fc polypeptide that comprises a knob mutation T366W and amino acid modification N434S with or without M428L, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, and hole mutations T366S, L368A, and Y407V, according to EU numbering scheme, and (b) a second Fc polypeptide that comprises a knob mutation T366W and amino acid modifications M252Y, S254T, and T256E, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, and hole mutations T366S, L368A, and Y407V, according to EU numbering scheme, and (b) a second Fc polypeptide that comprises a knob mutation T366W and amino acid modification N434S with or without M428L, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, hole mutations T366S, L368A, and Y407V, and amino acid modifications M252Y, S254T, and T256E, according to EU numbering scheme, and (b) a second Fc polypeptide that comprises a knob mutation T366W and amino acid modifications M252Y, S254T, and T256E, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A and L235A, hole mutations T366S, L368A, and Y407V, and amino acid modification N434S with or without M428L, according to EU numbering scheme, and (b) a second Fc polypeptide that comprises a knob mutation T366W and amino acid modification N434S with or without M428L, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, hole mutations T366S, L368A, and Y407V, and amino acid modifications M252Y, S254T, and T256E, according to EU numbering scheme, and (b) a second Fc polypeptide that comprises a knob mutation T366W and amino acid modifications M252Y, S254T, and T256E, according to EU numbering scheme, and does not contain a TfR-binding site or any modifications that reduce FcγR binding.

In another embodiment, an effector function-positive, TfR-binding Fc polypeptide dimer comprises: (a) a first Fc polypeptide comprising a TfR-binding site that specifically binds TfR, amino acid modifications L234A, L235A, and P329G, hole mutations T366S, L368A, and Y407V, and amino acid modification N434S with or without M428L, according to EU numbering scheme, and (b) a second Fc polypeptide that comprises a knob mutation T366W and amino acid modification N434S with or without M428L, according to EU numbering scheme, and does not contain a TfR-binding site or any modifications that reduce FcγR binding.

### VI. Measuring Effector Function or FcγR Binding

Methods for analyzing binding affinity, binding kinetics, and cross-reactivity between an Fc polypeptide dimer and FcγR are known in the art. These methods include, but are not limited to, solid-phase binding assays (e.g., ELISA assay), immunoprecipitation, surface plasmon resonance (e.g., Biacore™ (GE Healthcare, Piscataway, N.J.)), kinetic exclusion assays (e.g., KinExA®), flow cytometry, fluorescence-activated cell sorting (FACS), BioLayer interferometry (e.g., Octet® (ForteBio, Inc., Menlo Park, Calif.)), and Western blot analysis. In some embodiments, ELISA is used to determine binding affinity and/or cross-reactivity. Methods for performing ELISA assays are known in the art. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity.

ADCC is a type of immune response in which antibodies bind to antigens on the surface of pathogenic or tumorigenic target cells and identifies them for destruction by effector cells, e.g., peripheral blood mononuclear cells (e.g., natural killer (NK) cells, T cells, and B cells). Effector cells bearing FcγR recognize and bind the Fc region of the antibodies bound to the target cell. The antibodies thus confer specificity to the target cell killing. CDC is initiated when C1q, the initiating component of the classical complement pathway, is bound to the Fc region of target-bound antibodies. ADCC and CDC activities may be determined in a standard in vivo or in vitro assay of cell killing. Methods for determining ADCC and CDC activities are available in the art. In some embodiments, the methods may involve labeling target cells with a radioactive material, such as ⁵¹Cr, or a fluorescent dye, such as Calcein-AM. The labeled cells may be incubated with the antibody and effector cells and killing of the target cells by ADCC or CDC may be detected by the release of radioactivity or fluorescence.

Other assays to measure ADCC and CDC activities include, e.g., a lactate dehydrogenase (LDH) release assay. When the cell membranes are compromised or damaged in any way, LDH, a soluble yet stable enzyme in the cytoplasm, is released into the surrounding extracellular space. The presence of this enzyme in the culture medium can be used as a cell death marker. The relative amounts of live and dead cells within the medium can then be quantitated by measuring the amount of released LDH using a colorimetric or fluorometric LDH cytotoxicity assay.

### VII. Additional Mutations in an Fc Region that Comprises a Modified CH3 Domain Polypeptide

An Fc polypeptide as provided herein that is modified to bind TfR and initiate transport across the BBB may also comprise additional mutations, e.g., to increase serum stability or serum half-life, to modulate effector function, to influence glycosylation, to reduce immunogenicity in humans, and/or to provide for knob and hole heterodimerization of Fc polypeptides.

In some embodiments, a modified Fc polypeptide described herein has an amino acid sequence identity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a corresponding wild-type Fc polypeptide (e.g., a human IgG1, IgG2, IgG3, or IgG4 Fc polypeptide).

A modified Fc polypeptide described herein may also have other mutations introduced outside of the specified sets of amino acids, e.g., to influence glycosylation, to increase serum half-life or, for CH3 domains, to provide for knob and hole heterodimerization of polypeptides that comprise the modified CH3 domain. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). Such additional mutations are at a position in the polypeptide that does not have a negative effect on binding of the modified CH3 domain to the TfR.

In one illustrative embodiment of a knob and hole approach for dimerization, a position corresponding to position 136 of SEQ ID NO:1 of a first Fc polypeptide subunit to be dimerized has a tryptophan in place of a native threonine and a second Fc polypeptide subunit of the dimer has a valine at a position corresponding to position 177 of SEQ ID NO:1 in place of the native tyrosine. The second subunit of the Fc polypeptide may further comprise a substitution in which the native threonine at the position corresponding to position 136 of SEQ ID NO:1 is substituted with a serine and a native leucine at the position corresponding to position 138 of SEQ ID NO:1 is substituted with an alanine.

A modified Fc polypeptide as described herein may also be engineered to contain other modifications for heterodimerization, e.g., electrostatic engineering of contact residues within a CH3-CH3 interface that are naturally charged or hydrophobic patch modifications.

In some embodiments, modifications to enhance serum half-life may be introduced. For example, in some embodiments, a modified Fc polypeptide as described herein comprises a CH2 domain comprising a Tyr at a position corresponding to position 22 of SEQ ID NO:1, Thr at a position corresponding to 24 of SEQ ID NO:1, and Glu at a position corresponding to position 26 of SEQ ID NO:1. Alternatively, a modified Fc polypeptide as described herein may comprise M198L and N204S substitutions as numbered with reference to SEQ ID NO:1. Alternatively, a modified Fc polypeptide as described herein may comprise an N204S or N204A substitution as numbered with reference to SEQ ID NO:1.

### Illustrative Fc Polypeptides Comprising Additional Mutations

A modified Fc polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.35.23.2.1, and CH3C.35.23.1.1) may comprise additional mutations including a knob mutation (e.g., T136W as numbered with reference to SEQ ID NO:1), hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), and/or mutations that increase serum stability or serum half-life (e.g., (i) M22Y, S24T, and T26E as numbered with reference to SEQ ID NO:1, or (ii) N204S with or without M198L as numbered with reference to SEQ ID NO:1).

In some embodiments, a modified Fc polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.35.23.2.1, and CH3C.35.23.1.1) may have a knob mutation (e.g., T136W as numbered with reference to SEQ ID NO:1) and at least 85% identity, at least 90% identity, or at least 95% identity to the sequence of any one of SEQ ID NOS:4-29, 64-127, and 268-274 (e.g., SEQ ID NOS:66, 68, 94, 107-109, 119, and 268-270). In some embodiments, a modified Fc polypeptide having the sequence of any one of SEQ ID NOS:4-29, 64-127, and 268-274 (e.g., SEQ ID NOS:66, 68, 94, 107-109, 119, and 268-270) may be modified to have a knob mutation.

In some embodiments, a modified Fc polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.35.23.2.1, and CH3C.35.23.1.1) may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of any one of SEQ ID NOS:4-29, 64-127, and 268-274 (e.g., SEQ ID NOS:66, 68, 94, 107-109, 119, and 268-270). In some embodiments, a modified Fc polypeptide having the sequence of any one of SEQ ID NOS:4-29, 64-127, and 268-274 (e.g., SEQ ID NOS:66, 68, 94, 107-109, 119, and 268-270) may be modified to have a knob mutation and mutations that modulate effector function.

In some embodiments, a modified Fc polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.35.23.2.1, and CH3C.35.23.1.1) may have a knob mutation (e.g., T136W as numbered with reference to SEQ ID NO:1), mutations that increase serum stability or serum half-life (e.g., (i) M22Y, S24T, and T26E as numbered with reference to SEQ ID NO:1, or (ii) N204S with or without M198L as numbered with reference to SEQ ID NO:1), and at least 85% identity, at least 90% identity, or at least 95% identity to the sequence of any one of SEQ ID NOS:4-29, 64-127, and 268-274 (e.g., SEQ ID NOS:66, 68, 94, 107-109, 119, and 268-270). In some embodiments, a modified Fc polypeptide having the sequence of any one of SEQ ID NOS:4-29, 64-127, and 268-274 (e.g., SEQ ID NOS:66, 68, 94, 107-109, 119, and 268-270) may be modified to have a knob mutation and mutations that increase serum stability or serum half-life.

In some embodiments, a modified Fc polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.35.23.2.1, and CH3C.35.23.1.1) may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., (i) M22Y, S24T, and T26E as numbered with reference to SEQ ID NO:1, or (ii) N204S with or without M198L as numbered with reference to SEQ ID NO:1), and at least 85% identity, at least 90% identity, or at least 95% identity to the sequence of any one of SEQ ID NOS:4-29, 64-127, and 268-274 (e.g., SEQ ID NOS:66, 68, 94, 107-109, 119, and 268-270). In some embodiments, a modified Fc polypeptide having the sequence of any one of SEQ ID NOS:4-29, 64-127, and 268-274 (e.g., SEQ ID NOS:66, 68, 94, 107-109, 119, and 268-270) may be modified to have a knob mutation, mutations that modulate effector function, and mutations that increase serum stability or serum half-life.

In some embodiments, a modified Fc polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.35.23.2.1, and CH3C.35.23.1.1) may have hole mutations (e.g., T136S, L138A, and Y177V as numbered with reference to SEQ ID NO:1) and at least 85% identity, at least 90% identity, or at least 95% identity to the sequence of any one of SEQ ID NOS:4-29, 64-127, and 268-274 (e.g., SEQ ID NOS:66, 68, 94, 107-109, 119, and 268-270). In some embodiments, a modified Fc polypeptide having the sequence of any one of SEQ ID NOS:4-29, 64-127, and 268-274 (e.g., SEQ ID NOS:66, 68, 94, 107-109, 119, and 268-270) may be modified to have hole mutations.

In some embodiments, a modified Fc polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.35.23.2.1, and CH3C.35.23.1.1) may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of any one of SEQ ID NOS:4-29, 64-127, and 268-274 (e.g., SEQ ID NOS:66, 68, 94, 107-109, 119, and 268-270). In some embodiments, a modified Fc polypeptide having the sequence of any one of SEQ ID NOS:4-29, 64-127, and 268-274 (e.g., SEQ ID NOS:66, 68, 94, 107-109, 119, and 268-270) may be modified to have hole mutations and mutations that modulate effector function.

In some embodiments, a modified Fc polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.35.23.2.1, and CH3C.35.23.1.1) may have hole mutations (e.g., T136S, L138A, and Y177V as numbered with reference to SEQ ID NO:1), mutations that increase serum stability or serum half-life (e.g., (i) M22Y, S24T, and T26E as numbered with reference to SEQ ID NO:1, or (ii) N204S with or without M198L as numbered with reference to SEQ ID NO:1), and at least 85% identity, at least 90% identity, or at least 95% identity to the sequence of any one of SEQ ID NOS:4-29, 64-127, and 268-274 (e.g., SEQ ID NOS:66, 68, 94, 107-109, 119, and 268-270). In some embodiments, a modified Fc polypeptide having the sequence of any one of SEQ ID NOS:4-29, 64-127, and 268-274 (e.g., SEQ ID NOS:66, 68, 94, 107-109, 119, and 268-270) may be modified to have hole mutations and mutations that increase serum stability or serum half-life.

In some embodiments, a modified Fc polypeptide as described herein (e.g., any one of clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.35.23.2.1, and CH3C.35.23.1.1) may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., (i) M22Y, S24T, and T26E as numbered with reference to SEQ ID NO:1, or (ii) N204S with or without M198L as numbered with reference to SEQ ID NO:1), and at least 85% identity, at least 90% identity, or at least 95% identity to the sequence of any one of SEQ ID NOS:4-29, 64-127, and 268-274 (e.g., SEQ ID NOS:66, 68, 94, 107-109, 119, and 268-270). In some embodiments, a modified Fc polypeptide having the sequence of any one of SEQ ID NOS:4-29, 64-127, and 268-274 (e.g., SEQ ID NOS:66, 68, 94, 107-109, 119, and 268-270) may be modified to have hole mutations, mutations that modulate effector function, and mutations that increase serum stability or serum half-life.

Clone CH3C.35.20.1

In some embodiments, clone CH3C.35.20.1 may have a knob mutation (e.g., T136W as numbered with reference to SEQ ID NO:1) and at least 85% identity, at least 90% identity, or at least 95% identity to the sequence of SEQ ID NO:177. In some embodiments, clone CH3C.35.20.1 with the knob mutation has the sequence of SEQ ID NO:177.

In some embodiments, clone CH3C.35.20.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:178 or 179. In some embodiments, clone CH3C.35.20.1 with the knob mutation and the mutations that modulate effector function has the sequence of SEQ ID NO:178 or 179.

In some embodiments, clone CH3C.35.20.1 may have a knob mutation (e.g., T136W as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:180. In some embodiments, clone CH3C.35.20.1 with the knob mutation and the mutations that increase serum stability or serum half-life has the sequence of SEQ ID NO:180.

In some embodiments, clone CH3C.35.20.1 may have a knob mutation (e.g., T136W as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:322. In some embodiments, clone CH3C.35.20.1 with the knob mutation and the mutations that increase serum stability or serum half-life has the sequence of SEQ ID NO:322.

In some embodiments, clone CH3C.35.20.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:181 or 182. In some embodiments, clone CH3C.35.20.1 with the knob mutation, the mutations that modulate effector function, and the mutations that increase serum stability or serum half-life has the sequence of SEQ ID NO:181 or 182.

In some embodiments, clone CH3C.35.20.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:323 or 324. In some embodiments, clone CH3C.35.20.1 with the knob mutation, the mutations that modulate effector function, and the mutations that increase serum stability or serum half-life has the sequence of SEQ ID NO:323 or 324.

In some embodiments, clone CH3C.35.20.1 may have hole mutations (e.g., T136S, L138A, and Y177V as numbered with reference to SEQ ID NO:1) and at least 85% identity, at least 90% identity, or at least 95% identity to the sequence of SEQ ID NO:183. In some embodiments, clone CH3C.35.20.1 with the hole mutations has the sequence of SEQ ID NO:183.

In some embodiments, clone CH3C.35.20.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:184 or 185. In some embodiments, clone CH3C.35.20.1 with the hole mutations and the mutations that modulate effector function has the sequence of SEQ ID NO:184 or 185.

In some embodiments, clone CH3C.35.20.1 may have hole mutations (e.g., T136S, L138A, and Y177V as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:186. In some embodiments, clone CH3C.35.20.1 with the hole mutations and the mutations that increase serum stability or serum half-life has the sequence of SEQ ID NO:186.

In some embodiments, clone CH3C.35.20.1 may have hole mutations (e.g., T136S, L138A, and Y177V as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:325. In some embodiments, clone CH3C.35.20.1 with the hole mutations and the mutations that increase serum stability or serum half-life has the sequence of SEQ ID NO:325.

In some embodiments, clone CH3C.35.20.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:187 or 188. In some embodiments, clone CH3C.35.20.1 with the hole mutations, the mutations that modulate effector function, and the mutations that increase serum stability or serum half-life has the sequence of SEQ ID NO:187 or 188.

In some embodiments, clone CH3C.35.20.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:326 or 327. In some embodiments, clone CH3C.35.20.1 with the hole mutations, the mutations that modulate effector function, and the mutations that increase serum stability or serum half-life has the sequence of SEQ ID NO:326 or 327.

Clone CH3C.35.23.2

In some embodiments, clone CH3C.35.23.2 may have a knob mutation (e.g., T136W as numbered with reference to SEQ ID NO:1) and at least 85% identity, at least 90% identity, or at least 95% identity to the sequence of SEQ ID NO:189. In some embodiments, clone CH3C.35.23.2 with the knob mutation has the sequence of SEQ ID NO:189.

In some embodiments, clone CH3C.35.23.2 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:190 or 191. In some embodiments, clone CH3C.35.23.2 with the knob mutation and the mutations that modulate effector function has the sequence of SEQ ID NO:190 or 191.

In some embodiments, clone CH3C.35.23.2 may have a knob mutation (e.g., T136W as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:192. In some embodiments, clone CH3C.35.23.2 with the knob mutation and the mutations that increase serum stability or serum half-life has the sequence of SEQ ID NO:192.

In some embodiments, clone CH3C.35.23.2 may have a knob mutation (e.g., T136W as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:329. In some embodiments, clone CH3C.35.23.2 with the knob mutation and the mutations that increase serum stability or serum half-life has the sequence of SEQ ID NO:329.

In some embodiments, clone CH3C.35.23.2 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:193 or 194. In some embodiments, clone CH3C.35.23.2 with the knob mutation, the mutations that modulate effector function, and the mutations that increase serum stability or serum half-life has the sequence of SEQ ID NO:193 or 194.

In some embodiments, clone CH3C.35.23.2 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:330 or 331. In some embodiments, clone CH3C.35.23.2 with the knob mutation, the mutations that modulate effector function, and the mutations that increase serum stability or serum half-life has the sequence of SEQ ID NO:330 or 331.

In some embodiments, clone CH3C.35.23.2 may have hole mutations (e.g., T136S, L138A, and Y177V as numbered with reference to SEQ ID NO:1) and at least 85% identity, at least 90% identity, or at least 95% identity to the sequence of SEQ ID NO:195. In some embodiments, clone CH3C.35.23.2 with the hole mutations has the sequence of SEQ ID NO:195.

In some embodiments, clone CH3C.35.23.2 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:196 or 197. In some embodiments, clone CH3C.35.23.2 with the hole mutations and the mutations that modulate effector function has the sequence of SEQ ID NO:196 or 197.

In some embodiments, clone CH3C.35.23.2 may have hole mutations (e.g., T136S, L138A, and Y177V as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:198. In some embodiments, clone CH3C.35.23.2 with the hole mutations and the mutations that increase serum stability or serum half-life has the sequence of SEQ ID NO:198.

In some embodiments, clone CH3C.35.23.2 may have hole mutations (e.g., T136S, L138A, and Y177V as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:332. In some embodiments, clone CH3C.35.23.2 with the hole mutations and the mutations that increase serum stability or serum half-life has the sequence of SEQ ID NO:332.

In some embodiments, clone CH3C.35.23.2 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:199 or 200. In some embodiments, clone CH3C.35.23.2 with the hole mutations, the mutations that modulate effector function, and the mutations that increase serum stability or serum half-life has the sequence of SEQ ID NO:199 or 200.

In some embodiments, clone CH3C.35.23.2 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:333 or 334. In some embodiments, clone CH3C.35.23.2 with the hole mutations, the mutations that modulate effector function, and the mutations that increase serum stability or serum half-life has the sequence of SEQ ID NO:333 or 334.

Clone CH3C.35.23.3

In some embodiments, clone CH3C.35.23.3 may have a knob mutation (e.g., T136W as numbered with reference to SEQ ID NO:1) and at least 85% identity, at least 90% identity, or at least 95% identity to the sequence of SEQ ID NO:201. In some embodiments, clone CH3C.35.23.3 with the knob mutation has the sequence of SEQ ID NO:201.

In some embodiments, clone CH3C.35.23.3 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:202 or 203. In some embodiments, clone CH3C.35.23.3 with the knob mutation and the mutations that modulate effector function has the sequence of SEQ ID NO:202 or 203.

In some embodiments, clone CH3C.35.23.3 may have a knob mutation (e.g., T136W as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:204. In some embodiments, clone CH3C.35.23.3 with the knob mutation and the mutations that increase serum stability or serum half-life has the sequence of SEQ ID NO:204.

In some embodiments, clone CH3C.35.23.3 may have a knob mutation (e.g., T136W as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:336. In some embodiments, clone CH3C.35.23.3 with the knob mutation and the mutations that increase serum stability or serum half-life has the sequence of SEQ ID NO:336.

In some embodiments, clone CH3C.35.23.3 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:205 or 206. In some embodiments, clone CH3C.35.23.3 with the knob mutation, the mutations that modulate effector function, and the mutations that increase serum stability or serum half-life has the sequence of SEQ ID NO:205 or 206.

In some embodiments, clone CH3C.35.23.3 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:337 or 338. In some embodiments, clone CH3C.35.23.3 with the knob mutation, the mutations that modulate effector function, and the mutations that increase serum stability or serum half-life has the sequence of SEQ ID NO:337 or 338.

In some embodiments, clone CH3C.35.23.3 may have hole mutations (e.g., T136S, L138A, and Y177V as numbered with reference to SEQ ID NO:1) and at least 85% identity, at least 90% identity, or at least 95% identity to the sequence of SEQ ID NO:207. In some embodiments, clone CH3C.35.23.3 with the hole mutations and the sequence of SEQ ID NO:207.

In some embodiments, clone CH3C.35.23.3 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:208 or 209. In some embodiments, clone CH3C.35.23.3 with the hole mutations and the mutations that modulate effector function has the sequence of SEQ ID NO:208 or 209.

In some embodiments, clone CH3C.35.23.3 may have hole mutations (e.g., T136S, L138A, and Y177V as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:210. In some embodiments, clone CH3C.35.23.3 with the hole mutations and the mutations that increase serum stability or serum half-life has the sequence of SEQ ID NO:210.

In some embodiments, clone CH3C.35.23.3 may have hole mutations (e.g., T136S, L138A, and Y177V as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:339. In some embodiments, clone CH3C.35.23.3 with the hole mutations and the mutations that increase serum stability or serum half-life has the sequence of SEQ ID NO:339.

In some embodiments, clone CH3C.35.23.3 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:211 or 212. In some embodiments, clone CH3C.35.23.3 with the hole mutations, the mutations that modulate effector function, and the mutations that increase serum stability or serum half-life has the sequence of SEQ ID NO:211 or 212.

In some embodiments, clone CH3C.35.23.3 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:340 or 341. In some embodiments, clone CH3C.35.23.3 with the hole mutations, the mutations that modulate effector function, and the mutations that increase serum stability or serum half-life has the sequence of SEQ ID NO:340 or 341.

Clone CH3C. 35.23.4

In some embodiments, clone CH3C.35.23.4 may have a knob mutation (e.g., T136W as numbered with reference to SEQ ID NO:1) and at least 85% identity, at least 90% identity, or at least 95% identity to the sequence of SEQ ID NO:213. In some embodiments, clone CH3C.35.23.4 with the knob mutation has the sequence of SEQ ID NO:213.

In some embodiments, clone CH3C.35.23.4 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:214 or 215. In some embodiments, clone CH3C.35.23.4 with the knob mutation and the mutations that modulate effector function has the sequence of SEQ ID NO:214 or 215.

In some embodiments, clone CH3C.35.23.4 may have a knob mutation (e.g., T136W as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:216. In some embodiments, clone CH3C.35.23.4 with the knob mutation and the mutations that increase serum stability or serum half-life has the sequence of SEQ ID NO:216.

In some embodiments, clone CH3C.35.23.4 may have a knob mutation (e.g., T136W as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:343. In some embodiments, clone CH3C.35.23.4 with the knob mutation and the mutations that increase serum stability or serum half-life has the sequence of SEQ ID NO:343.

In some embodiments, clone CH3C.35.23.4 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:217 or 218. In some embodiments, clone CH3C.35.23.4 with the knob mutation, the mutations that modulate effector function, and the mutations that increase serum stability or serum half-life has the sequence of SEQ ID NO:217 or 218.

In some embodiments, clone CH3C.35.23.4 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:344 or 345. In some embodiments, clone CH3C.35.23.4 with the knob mutation, the mutations that modulate effector function, and the mutations that increase serum stability or serum half-life has the sequence of SEQ ID NO:344 or 345.

In some embodiments, clone CH3C.35.23.4 may have hole mutations (e.g., T136S, L138A, and Y177V as numbered with reference to SEQ ID NO:1) and at least 85% identity, at least 90% identity, or at least 95% identity to the sequence of SEQ ID NO:219. In some embodiments, clone CH3C.35.23.4 with the hole mutations has the sequence of SEQ ID NO:219.

In some embodiments, clone CH3C.35.23.4 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:220 or 221. In some embodiments, clone CH3C.35.23.4 with the hole mutations and the mutations that modulate effector function has the sequence of SEQ ID NO:220 or 221.

In some embodiments, clone CH3C.35.23.4 may have hole mutations (e.g., T136S, L138A, and Y177V as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:222. In some embodiments, clone CH3C.35.23.4 with the hole mutations and the mutations that increase serum stability or serum half-life has the sequence of SEQ ID NO:222.

In some embodiments, clone CH3C.35.23.4 may have hole mutations (e.g., T136S, L138A, and Y177V as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:346. In some embodiments, clone CH3C.35.23.4 with the hole mutations and the mutations that increase serum stability or serum half-life has the sequence of SEQ ID NO:346.

In some embodiments, clone CH3C.35.23.4 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:223 or 224. In some embodiments, clone CH3C.35.23.4 with the hole mutations, the mutations that modulate effector function, and the mutations that increase serum stability or serum half-life has the sequence of SEQ ID NO:223 or 224.

In some embodiments, clone CH3C.35.23.4 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:347 or 348. In some embodiments, clone CH3C.35.23.4 with the hole mutations, the mutations that modulate effector function, and the mutations that increase serum stability or serum half-life has the sequence of SEQ ID NO:347 or 348.

Clone CH3C.35.21.17.2

In some embodiments, clone CH3C.35.21.17.2 may have a knob mutation (e.g., T136W as numbered with reference to SEQ ID NO:1) and at least 85% identity, at least 90% identity, or at least 95% identity to the sequence of SEQ ID NO:225. In some embodiments, clone CH3C.35.21.17.2 with the knob mutation has the sequence of SEQ ID NO:225.

In some embodiments, clone CH3C.35.21.17.2 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:226 or 227. In some embodiments, clone CH3C.35.21.17.2 with the knob mutation and the mutations that modulate effector function has the sequence of SEQ ID NO:226 or 227.

In some embodiments, clone CH3C.35.21.17.2 may have a knob mutation (e.g., T136W as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:228. In some embodiments, clone CH3C.35.21.17.2 with the knob mutation and the mutations that increase serum stability or serum half-life has the sequence of SEQ ID NO:228.

In some embodiments, clone CH3C.35.21.17.2 may have a knob mutation (e.g., T136W as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:350. In some embodiments, clone CH3C.35.21.17.2 with the knob mutation and the mutations that increase serum stability or serum half-life has the sequence of SEQ ID NO:350.

In some embodiments, clone CH3C.35.21.17.2 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:229 or 230. In some embodiments, clone CH3C.35.21.17.2 with the knob mutation, the mutations that modulate effector function, and the mutations that increase serum stability or serum half-life has the sequence of SEQ ID NO:229 or 230.

In some embodiments, clone CH3C.35.21.17.2 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:351 or 352. In some embodiments, clone CH3C.35.21.17.2 with the knob mutation, the mutations that modulate effector function, and the mutations that increase serum stability or serum half-life has the sequence of SEQ ID NO:351 or 352.

In some embodiments, clone CH3C.35.21.17.2 may have hole mutations (e.g., T136S, L138A, and Y177V as numbered with reference to SEQ ID NO:1) and at least 85% identity, at least 90% identity, or at least 95% identity to the sequence of SEQ ID NO:231. In some embodiments, clone CH3C.35.21.17.2 with the hole mutations has the sequence of SEQ ID NO:231.

In some embodiments, clone CH3C.35.21.17.2 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:232 or 233. In some embodiments, clone CH3C.35.21.17.2 with the hole mutations and the mutations that modulate effector function has the sequence of SEQ ID NO:232 or 233.

In some embodiments, clone CH3C.35.21.17.2 may have hole mutations (e.g., T136S, L138A, and Y177V as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:234. In some embodiments, clone CH3C.35.21.17.2 with the hole mutations and the mutations that increase serum stability or serum half-life has the sequence of SEQ ID NO:234.

In some embodiments, clone CH3C.35.21.17.2 may have hole mutations (e.g., T136S, L138A, and Y177V as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:353. In some embodiments, clone CH3C.35.21.17.2 with the hole mutations and the mutations that increase serum stability or serum half-life has the sequence of SEQ ID NO:353.

In some embodiments, clone CH3C.35.21.17.2 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:235 or 236. In some embodiments, clone CH3C.35.21.17.2 with the hole mutations, the mutations that modulate effector function, and the mutations that increase serum stability or serum half-life has the sequence of SEQ ID NO:235 or 236.

In some embodiments, clone CH3C.35.21.17.2 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:354 or 355. In some embodiments, clone CH3C.35.21.17.2 with the hole mutations, the mutations that modulate effector function, and the mutations that increase serum stability or serum half-life has the sequence of SEQ ID NO:354 or 355.

Clone CH3C.35.23

In some embodiments, clone CH3C.35.23 may have a knob mutation (e.g., T136W as numbered with reference to SEQ ID NO:1) and at least 85% identity, at least 90% identity, or at least 95% identity to the sequence of SEQ ID NO:237. In some embodiments, clone CH3C.35.23 with the knob mutation has the sequence of SEQ ID NO:237.

In some embodiments, clone CH3C.35.23 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:238 or 239. In some embodiments, clone CH3C.35.23 with the knob mutation and the mutations that modulate effector function has the sequence of SEQ ID NO:238 or 239.

In some embodiments, clone CH3C.35.23 may have a knob mutation (e.g., T136W as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:240. In some embodiments, clone CH3C.35.23 with the knob mutation and the mutations that increase serum stability or serum half-life has the sequence of SEQ ID NO:240.

In some embodiments, clone CH3C.35.23 may have a knob mutation (e.g., T136W as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:357. In some embodiments, clone CH3C.35.23 with the knob mutation and the mutations that increase serum stability or serum half-life has the sequence of SEQ ID NO:357.

In some embodiments, clone CH3C.35.23 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:241 or 242. In some embodiments, clone CH3C.35.23 with the knob mutation, the mutations that modulate effector function, and the mutations that increase serum stability or serum half-life has the sequence of SEQ ID NO:241 or 242.

In some embodiments, clone CH3C.35.23 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:358 or 359. In some embodiments, clone CH3C.35.23 with the knob mutation, the mutations that modulate effector function, and the mutations that increase serum stability or serum half-life has the sequence of SEQ ID NO:358 or 359.

In some embodiments, clone CH3C.35.23 may have hole mutations (e.g., T136S, L138A, and Y177V as numbered with reference to SEQ ID NO:1) and at least 85% identity, at least 90% identity, or at least 95% identity to the sequence of SEQ ID NO:243. In some embodiments, clone CH3C.35.23 with the hole mutations has the sequence of SEQ ID NO:243.

In some embodiments, clone CH3C.35.23 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:244 or 245. In some embodiments, clone CH3C.35.23 with the hole mutations and the mutations that modulate effector function has the sequence of SEQ ID NO:244 or 245.

In some embodiments, clone CH3C.35.23 may have hole mutations (e.g., T136S, L138A, and Y177V as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:246. In some embodiments, clone CH3C.35.23 with the hole mutations and the mutations that increase serum stability or serum half-life has the sequence of SEQ ID NO:246.

In some embodiments, clone CH3C.35.23 may have hole mutations (e.g., T136S, L138A, and Y177V as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:360. In some embodiments, clone CH3C.35.23 with the hole mutations and the mutations that increase serum stability or serum half-life has the sequence of SEQ ID NO:360.

In some embodiments, clone CH3C.35.23 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:247 or 248. In some embodiments, clone CH3C.35.23 with the hole mutations, the mutations that modulate effector function, and the mutations that increase serum stability or serum half-life has the sequence of SEQ ID NO:247 or 248.

In some embodiments, clone CH3C.35.23 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:361 or 362. In some embodiments, clone CH3C.35.23 with the hole mutations, the mutations that modulate effector function, and the mutations that increase serum stability or serum half-life has the sequence of SEQ ID NO:361 or 362.

Clone CH3C.35.21

In some embodiments, clone CH3C.35.21 may have a knob mutation (e.g., T136W as numbered with reference to SEQ ID NO:1) and at least 85% identity, at least 90% identity, or at least 95% identity to the sequence of SEQ ID NO:250. In some embodiments, clone CH3C.35.21 with the knob mutation has the sequence of SEQ ID NO:250.

In some embodiments, clone CH3C.35.21 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:252 or 275. In some embodiments, clone CH3C.35.21 with the knob mutation and the mutations that modulate effector function has the sequence of SEQ ID NO:252 or 275.

In some embodiments, clone CH3C.35.21 may have a knob mutation (e.g., T136W as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:276. In some embodiments, clone CH3C.35.21 with the knob mutation and the mutations that increase serum stability or serum half-life has the sequence of SEQ ID NO:276.

In some embodiments, clone CH3C.35.21 may have a knob mutation (e.g., T136W as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:364. In some embodiments, clone CH3C.35.21 with the knob mutation and the mutations that increase serum stability or serum half-life has the sequence of SEQ ID NO:364.

In some embodiments, clone CH3C.35.21 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:277 or 278. In some embodiments, clone CH3C.35.21 with the knob mutation, the mutations that modulate effector function, and the mutations that increase serum stability or serum half-life has the sequence of SEQ ID NO:277 or 278.

In some embodiments, clone CH3C.35.21 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:365 or 366. In some embodiments, clone CH3C.35.21 with the knob mutation, the mutations that modulate effector function, and the mutations that increase serum stability or serum half-life has the sequence of SEQ ID NO:365 or 366.

In some embodiments, clone CH3C.35.21 may have hole mutations (e.g., T136S, L138A, and Y177V as numbered with reference to SEQ ID NO:1) and at least 85% identity, at least 90% identity, or at least 95% identity to the sequence of SEQ ID NO:279. In some embodiments, clone CH3C.35.21 with the hole mutations has the sequence of SEQ ID NO:279.

In some embodiments, clone CH3C.35.21 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:280 or 281. In some embodiments, clone CH3C.35.21 with the hole mutations and the mutations that modulate effector function has the sequence of SEQ ID NO:280 or 281.

In some embodiments, clone CH3C.35.21 may have hole mutations (e.g., T136S, L138A, and Y177V as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:282. In some embodiments, clone CH3C.35.21 with the hole mutations and the mutations that increase serum stability or serum half-life has the sequence of SEQ ID NO:282.

In some embodiments, clone CH3C.35.21 may have hole mutations (e.g., T136S, L138A, and Y177V as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:367. In some embodiments, clone CH3C.35.21 with the hole mutations and the mutations that increase serum stability or serum half-life has the sequence of SEQ ID NO:367.

In some embodiments, clone CH3C.35.21 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:283 or 284. In some embodiments, clone CH3C.35.21 with the hole mutations, the mutations that modulate effector function, and the mutations that increase serum stability or serum half-life has the sequence of SEQ ID NO:283 or 284.

In some embodiments, clone CH3C.35.21 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:368 or 369. In some embodiments, clone CH3C.35.21 with the hole mutations, the mutations that modulate effector function, and the mutations that increase serum stability or serum half-life has the sequence of SEQ ID NO:368 or 369.

Clone CH3C.35.20.1.1

In some embodiments, clone CH3C.35.20.1.1 may have a knob mutation (e.g., T136W as numbered with reference to SEQ ID NO:1) and at least 85% identity, at least 90% identity, or at least 95% identity to the sequence of SEQ ID NO:285. In some embodiments, clone CH3C.35.20.1.1 with the knob mutation has the sequence of SEQ ID NO:285.

In some embodiments, clone CH3C.35.20.1.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:286 or 287. In some embodiments, clone CH3C.35.20.1.1 with the knob mutation and the mutations that modulate effector function has the sequence of SEQ ID NO:286 or 287.

In some embodiments, clone CH3C.35.20.1.1 may have a knob mutation (e.g., T136W as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:288. In some embodiments, clone CH3C.35.20.1.1 with the knob mutation and the mutations that increase serum stability or serum half-life has the sequence of SEQ ID NO:288.

In some embodiments, clone CH3C.35.20.1.1 may have a knob mutation (e.g., T136W as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:371. In some embodiments, clone CH3C.35.20.1.1 with the knob mutation and the mutations that increase serum stability or serum half-life has the sequence of SEQ ID NO:371.

In some embodiments, clone CH3C.35.20.1.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:289 or 290. In some embodiments, clone CH3C.35.20.1.1 with the knob mutation, the mutations that modulate effector function, and the mutations that increase serum stability or serum half-life has the sequence of SEQ ID NO:289 or 290.

In some embodiments, clone CH3C.35.20.1.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:372 or 373. In some embodiments, clone CH3C.35.20.1.1 with the knob mutation, the mutations that modulate effector function, and the mutations that increase serum stability or serum half-life has the sequence of SEQ ID NO:372 or 373.

In some embodiments, clone CH3C.35.20.1.1 may have hole mutations (e.g., T136S, L138A, and Y177V as numbered with reference to SEQ ID NO:1) and at least 85% identity, at least 90% identity, or at least 95% identity to the sequence of SEQ ID NO:291. In some embodiments, clone CH3C.35.20.1.1 with the hole mutations has the sequence of SEQ ID NO:291.

In some embodiments, clone CH3C.35.20.1.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:292 or 293. In some embodiments, clone CH3C.35.20.1.1 with the hole mutations and the mutations that modulate effector function has the sequence of SEQ ID NO:292 or 293.

In some embodiments, clone CH3C.35.20.1.1 may have hole mutations (e.g., T136S, L138A, and Y177V as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:294. In some embodiments, clone CH3C.35.20.1.1 with the hole mutations and the mutations that increase serum stability or serum half-life has the sequence of SEQ ID NO:294.

In some embodiments, clone CH3C.35.20.1.1 may have hole mutations (e.g., T136S, L138A, and Y177V as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:374. In some embodiments, clone CH3C.35.20.1.1 with the hole mutations and the mutations that increase serum stability or serum half-life has the sequence of SEQ ID NO:374.

In some embodiments, clone CH3C.35.20.1.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:295 or 296. In some embodiments, clone CH3C.35.20.1.1 with the hole mutations, the mutations that modulate effector function, and the mutations that increase serum stability or serum half-life has the sequence of SEQ ID NO:295 or 296.

In some embodiments, clone CH3C.35.20.1.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:375 or 376. In some embodiments, clone CH3C.35.20.1.1 with the hole mutations, the mutations that modulate effector function, and the mutations that increase serum stability or serum half-life has the sequence of SEQ ID NO:375 or 376.

Clone CH3C.35.23.2.1

In some embodiments, clone CH3C.35.23.2.1 may have a knob mutation (e.g., T136W as numbered with reference to SEQ ID NO:1) and at least 85% identity, at least 90% identity, or at least 95% identity to the sequence of SEQ ID NO:297. In some embodiments, clone CH3C.35.23.2.1 with the knob mutation has the sequence of SEQ ID NO:297.

In some embodiments, clone CH3C.35.23.2.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:298 or 299. In some embodiments, clone CH3C.35.23.2.1 with the knob mutation and the mutations that modulate effector function has the sequence of SEQ ID NO:298 or 299.

In some embodiments, clone CH3C.35.23.2.1 may have a knob mutation (e.g., T136W as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:300. In some embodiments, clone CH3C.35.23.2.1 with the knob mutation and the mutations that increase serum stability or serum half-life has the sequence of SEQ ID NO:300.

In some embodiments, clone CH3C.35.23.2.1 may have a knob mutation (e.g., T136W as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:378. In some embodiments, clone CH3C.35.23.2.1 with the knob mutation and the mutations that increase serum stability or serum half-life has the sequence of SEQ ID NO:378.

In some embodiments, clone CH3C.35.23.2.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:301 or 302. In some embodiments, clone CH3C.35.23.2.1 with the knob mutation, the mutations that modulate effector function, and the mutations that increase serum stability or serum half-life has the sequence of SEQ ID NO:301 or 302.

In some embodiments, clone CH3C.35.23.2.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:379 or 380. In some embodiments, clone CH3C.35.23.2.1 with the knob mutation, the mutations that modulate effector function, and the mutations that increase serum stability or serum half-life has the sequence of SEQ ID NO:379 or 380.

In some embodiments, clone CH3C.35.23.2.1 may have hole mutations (e.g., T136S, L138A, and Y177V as numbered with reference to SEQ ID NO:1) and at least 85% identity, at least 90% identity, or at least 95% identity to the sequence of SEQ ID NO:303. In some embodiments, clone CH3C.35.23.2.1 with the hole mutations has the sequence of SEQ ID NO:303.

In some embodiments, clone CH3C.35.23.2.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:304 or 305. In some embodiments, clone CH3C.35.23.2.1 with the hole mutations and the mutations that modulate effector function has the sequence of SEQ ID NO:304 or 305.

In some embodiments, clone CH3C.35.23.2.1 may have hole mutations (e.g., T136S, L138A, and Y177V as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:306. In some embodiments, clone CH3C.35.23.2.1 with the hole mutations and the mutations that increase serum stability or serum half-life has the sequence of SEQ ID NO:306.

In some embodiments, clone CH3C.35.23.2.1 may have hole mutations (e.g., T136S, L138A, and Y177V as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:381. In some embodiments, clone CH3C.35.23.2.1 with the hole mutations and the mutations that increase serum stability or serum half-life has the sequence of SEQ ID NO:381.

In some embodiments, clone CH3C.35.23.2.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:307 or 308. In some embodiments, clone CH3C.35.23.2.1 with the hole mutations, the mutations that modulate effector function, and the mutations that increase serum stability or serum half-life has the sequence of SEQ ID NO:307 or 308.

In some embodiments, clone CH3C.35.23.2.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:382 or 383. In some embodiments, clone CH3C.35.23.2.1 with the hole mutations, the mutations that modulate effector function, and the mutations that increase serum stability or serum half-life has the sequence of SEQ ID NO:382 or 383.

Clone CH3C.35.23.1.1

In some embodiments, clone CH3C.35.23.1.1 may have a knob mutation (e.g., T136W as numbered with reference to SEQ ID NO:1) and at least 85% identity, at least 90% identity, or at least 95% identity to the sequence of SEQ ID NO:309. In some embodiments, clone CH3C.35.23.1.1 with the knob mutation has the sequence of SEQ ID NO:309.

In some embodiments, clone CH3C.35.23.1.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:310 or 311. In some embodiments, clone CH3C.35.23.1.1 with the knob mutation and the mutations that modulate effector function has the sequence of SEQ ID NO:310 or 311.

In some embodiments, clone CH3C.35.23.1.1 may have a knob mutation (e.g., T136W as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:312. In some embodiments, clone CH3C.35.23.1.1 with the knob mutation and the mutations that increase serum stability or serum half-life has the sequence of SEQ ID NO:312.

In some embodiments, clone CH3C.35.23.1.1 may have a knob mutation (e.g., T136W as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:385. In some embodiments, clone CH3C.35.23.1.1 with the knob mutation and the mutations that increase serum stability or serum half-life has the sequence of SEQ ID NO:385.

In some embodiments, clone CH3C.35.23.1.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:313 or 314. In some embodiments, clone CH3C.35.23.1.1 with the knob mutation, the mutations that modulate effector function, and the mutations that increase serum stability or serum half-life has the sequence of SEQ ID NO:313 or 314.

In some embodiments, clone CH3C.35.23.1.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:386 or 387. In some embodiments, clone CH3C.35.23.1.1 with the knob mutation, the mutations that modulate effector function, and the mutations that increase serum stability or serum half-life has the sequence of SEQ ID NO:386 or 387.

In some embodiments, clone CH3C.35.23.1.1 may have hole mutations (e.g., T136S, L138A, and Y177V as numbered with reference to SEQ ID NO:1) and at least 85% identity, at least 90% identity, or at least 95% identity to the sequence of SEQ ID NO:315. In some embodiments, clone CH3C.35.23.1.1 with the hole mutations has the sequence of SEQ ID NO:315.

In some embodiments, clone CH3C.35.23.1.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:316 or 317. In some embodiments, clone CH3C.35.23.1.1 with the hole mutations and the mutations that modulate effector function has the sequence of SEQ ID NO:316 or 317.

In some embodiments, clone CH3C.35.23.1.1 may have hole mutations (e.g., T136S, L138A, and Y177V as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:318. In some embodiments, clone CH3C.35.23.1.1 with the hole mutations and the mutations that increase serum stability or serum half-life has the sequence of SEQ ID NO:318.

In some embodiments, clone CH3C.35.23.1.1 may have hole mutations (e.g., T136S, L138A, and Y177V as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:388. In some embodiments, clone CH3C.35.23.1.1 with the hole mutations and the mutations that increase serum stability or serum half-life has the sequence of SEQ ID NO:388.

In some embodiments, clone CH3C.35.23.1.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:319 or 320. In some embodiments, clone CH3C.35.23.1.1 with the hole mutations, the mutations that modulate effector function, and the mutations that increase serum stability or serum half-life has the sequence of SEQ ID NO:319 or 320.

In some embodiments, clone CH3C.35.23.1.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:389 or 390. In some embodiments, clone CH3C.35.23.1.1 with the hole mutations, the mutations that modulate effector function, and the mutations that increase serum stability or serum half-life has the sequence of SEQ ID NO:389 or 390.

### VIII. Formats for TfR-Binding Proteins

In some embodiments, a modified TfR-binding polypeptide as described herein is a subunit of a protein dimer. In some embodiments, the dimer is a heterodimer. In some embodiments, the dimer is a homodimer. In some embodiments, the dimer comprises a single Fc polypeptide that binds to the TfR receptor, i.e., is monovalent for TfR receptor binding. In some embodiments, the dimer comprises a second polypeptide that binds to the TfR receptor. The second polypeptide may comprise the same modified Fc polypeptide to provide a bivalent homodimer protein, or a second modified Fc polypeptide described herein may provide a second TfR receptor-binding site.

TfR-binding polypeptides described herein and dimeric or multimeric proteins comprising polypeptides may have a broad range of binding affinities, e.g., based on the format of the polypeptide. For example, in some embodiments, a polypeptide comprising a modified Fc polypeptide as described herein has an affinity for the TfR ranging anywhere from 1 pM to 10 μM. In some embodiments, affinity may be measured in a monovalent format. In other embodiments, affinity may be measured in a bivalent format, e.g., as a protein dimer comprising a modified Fc polypeptide.

Methods for analyzing binding affinity, binding kinetics, and cross-reactivity to analyze binding to TfR are known in the art. These methods include, but are not limited to, solid-phase binding assays (e.g., ELISA assay), immunoprecipitation, surface plasmon resonance (e.g., Biacore™ (GE Healthcare, Piscataway, N.J.)), kinetic exclusion assays (e.g., KinExA®), flow cytometry, fluorescence-activated cell sorting (FACS), BioLayer interferometry (e.g., Octet® (FortéBio, Inc., Menlo Park, Calif.)), and Western blot analysis. In some embodiments, ELISA is used to determine binding affinity and/or cross-reactivity. Methods for performing ELISA assays are known in the art and are also described in the Example section below. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity. FcRn binding of TfR-binding polypeptide may also be evaluated using these types of assays. FcRn binding is typically assayed under acidic conditions, e.g., at a pH of about 5 to about 6.

### IX. TfR-Binding Protein Conjugates

In some embodiments, a modified polypeptide that binds TfR and initiates transport across the BBB comprises a modified Fc polypeptide as described herein and further comprises a partial or full hinge region. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:62). In further embodiments, the polypeptide, which may comprise a hinge or partial hinge region, is further fused to another moiety, for example, an immunoglobulin variable region, thus generating a TfR-binding polypeptide-variable region fusion polypeptide. The variable region may bind to any antigen of interest, e.g., a therapeutic neurological target, or a diagnostic neurological target.

In some embodiments, the TfR-binding polypeptide (e.g., modified Fc polypeptide) is fused to a variable region via a linker. As indicated in the preceding paragraph, the TfR-binding polypeptide (e.g., modified Fc polypeptide) may be fused to the variable region by a hinge region. In some embodiments, the TfR-binding polypeptide (e.g., modified Fc polypeptide) may be fused to the variable region by a peptide linker. The peptide linker may be configured such that it allows for the rotation of the variable region and the TfR-binding polypeptide relative to each other; and/or is resistant to digestion by proteases. In some embodiments, the linker may be a flexible linker, e.g., containing amino acids such as Gly, Asn, Ser, Thr, Ala, and the like. Such linkers are designed using known parameters. For example, the linker may have repeats, such as Gly-Ser repeats.

The variable region may be in any antibody format, e.g., a Fab or scFv format. In some embodiments, an antibody variable region sequence comprises two antibody variable region heavy chains and two antibody variable region light chains, or respective fragments thereof.

A TfR-binding polypeptide (e.g., modified Fc polypeptide) may also be fused to a polypeptide other than an immunoglobulin variable region that targets an antigen of interest. In some embodiments, such a polypeptide is fused to the TfR-binding polypeptide using a peptide linker, e.g., a flexible linker, as described above.

In some embodiments, a TfR-binding polypeptide may be fused to a polypeptide, e.g., a therapeutic polypeptide, that is desirable to target to a cell expressing the TfR-binding polypeptide. In some embodiments, the TfR-binding polypeptide is fused to a biologically active polypeptide for transport across the BBB, e.g., a soluble protein, e.g., an extracellular domain of a receptor or a growth factor, a cytokine, or an enzyme.

In still other embodiments, the TfR-binding polypeptide may be fused to a peptide or protein useful in protein purification, e.g., polyhistidine, epitope tags, e.g., FLAG, c-Myc, hemagglutinin tags and the like, glutathione S transferase (GST), thioredoxin, protein A, protein G, or maltose binding protein (MBP). In some cases, the peptide or protein to which the TfR-binding polypeptide is fused may comprise a protease cleavage site, such as a cleavage site for Factor Xa or Thrombin. In certain embodiments, the linkage is cleavable by an enzyme present in the central nervous system.

Non-polypeptide agents may also be attached to a TfR-binding polypeptide. Such agents include cytotoxic agents, imaging agents, a DNA or RNA molecule, or a chemical compound. In some embodiments, the agent may be a therapeutic or imaging chemical compound. In some embodiments, the agent is a small molecule, e.g., less than 1000 Da, less than 750 Da, or less than 500 Da.

An agent, either a polypeptide or non-polypeptide, may be attached to the N-terminal or C-terminal region of the TfR-binding polypeptide, or attached to any region of the polypeptide, so long as the agent does not interfere with binding of the TfR-binding polypeptide to TfR.

In various embodiments, the conjugates can be generated using well-known chemical cross-linking reagents and protocols. For example, there are a large number of chemical cross-linking agents that are known to those skilled in the art and useful for cross-linking the polypeptide with an agent of interest. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers.

The agent of interest may be a therapeutic agent, including cytotoxic agents and the like, or a chemical moiety. In some embodiments, the agent may be a peptide or small molecule therapeutic or imaging agent.

### X. Methods to Increase Effector Function

For some applications, it is desirable to introduce modifications into modified Fc polypeptides or modified Fc polypeptide dimers described herein that increase effector function (e.g., ADCC). One method for increasing effector function involves producing modified Fc polypeptides or modified Fc polypeptide dimers that are afucosylated or fucose-deficient.

One approach for generating fucose-deficient modified Fc polypeptides or modified Fc polypeptide dimers is to use a fucose analog such as 2-fluorofucose (2-FF). Fucose analogs can deplete or decrease the availability of GDP-fucose, which is a substrate required by fucosyltransferases to incorporate fucose into proteins.

An alternative approach for generating fucose-deficient modified Fc polypeptides or modified Fc polypeptide dimers, commonly used for commercial production, is to employ an alpha-1,6 fucosyltransferase (FUT8) knockout cell line for expression of the modified Fc polypeptides or modified Fc polypeptide dimers. A non-limiting example of a suitable FUT8 knockout cell line is the Chinese hamster ovary (CHO) FUT8 knockout cell line available from Lonza Biologics. Furthermore, as described in Mori et al. (Biotechnol. Bioeng. (2004) 88:901-908; hereby incorporated by reference in its entirety), FUT8 small interfering RNA (siRNA) can be used to convert CHO cell lines (e.g., by constitutive expression of the FUT8 siRNA) for the production of fucose-deficient proteins.

### XI. Nucleic Acids, Vectors, and Host Cells

Modified TfR-binding polypeptides as described herein are typically prepared using recombinant methods. Accordingly, isolated nucleic acids comprising a nucleic acid sequence encoding any of the polypeptides comprising modified Fc polypeptides as described herein, and host cells into which the nucleic acids are introduced that are used to replicate the polypeptide-encoding nucleic acids and/or to express the polypeptides. In some embodiments, the host cell is eukaryotic, e.g., a human cell.

In another aspect, polynucleotides are provided that comprise a nucleotide sequence that encodes the polypeptides described herein. The polynucleotides may be single-stranded or double-stranded. In some embodiments, the polynucleotide is DNA. In particular embodiments, the polynucleotide is cDNA. In some embodiments, the polynucleotide is RNA.

In some embodiments, the polynucleotide is included within a nucleic acid construct. In some embodiments, the construct is a replicable vector. In some embodiments, the vector is selected from a plasmid, a viral vector, a phagemid, a yeast chromosomal vector, and a non-episomal mammalian vector.

In some embodiments, the polynucleotide is operably linked to one or more regulatory nucleotide sequences in an expression construct. In one series of embodiments, the nucleic acid expression constructs are adapted for use as a surface expression library. In some embodiments, the library is adapted for surface expression in yeast. In some embodiments, the library is adapted for surface expression in phage. In another series of embodiments, the nucleic acid expression constructs are adapted for expression of the polypeptide in a system that permits isolation of the polypeptide in milligram or gram quantities. In some embodiments, the system is a mammalian cell expression system. In some embodiments, the system is a yeast cell expression system.

Expression vehicles for production of a recombinant polypeptide include plasmids and other vectors. For instance, suitable vectors include plasmids of the following types: pBR322-derived plasmids, pEMBL-derived plasmids, pEX-derived plasmids, pBTac-derived plasmids, and pUC-derived plasmids for expression in prokaryotic cells, such as E. coli. The pcDNAI/amp, pcDNAI/neo, pRc/CMV, pSV2gpt, pSV2neo, pSV2-dhfr, pTk2, pRSVneo, pMSG, pSVT7, pko-neo, and pHyg-derived vectors are examples of mammalian expression vectors suitable for transfection of eukaryotic cells. Alternatively, derivatives of viruses such as the bovine papilloma virus (BPV-1), or Epstein-Barr virus (pHEBo, pREP-derived, and p205) can be used for transient expression of polypeptides in eukaryotic cells. In some embodiments, it may be desirable to express the recombinant polypeptide by the use of a baculovirus expression system. Examples of such baculovirus expression systems include pVL-derived vectors (such as pVL1392, pVL1393, and pVL941), pAcUW-derived vectors (such as pAcUW1), and pBlueBac-derived vectors. Additional expression systems include adenoviral, adeno-associated virus, and other viral expression systems.

Vectors may be transformed into any suitable host cell. In some embodiments, the host cells, e.g., bacteria or yeast cells, may be adapted for use as a surface expression library. In some cells, the vectors are expressed in host cells to express relatively large quantities of the polypeptide. Such host cells include mammalian cells, yeast cells, insect cells, and prokaryotic cells. In some embodiments, the cells are mammalian cells, such as Chinese Hamster Ovary (CHO) cell, baby hamster kidney (BHK) cell, NS0 cell, Y0 cell, HEK293 cell, COS cell, Vero cell, or HeLa cell.

A host cell transfected with an expression vector encoding a TfR-binding polypeptide can be cultured under appropriate conditions to allow expression of the polypeptide to occur. The polypeptides may be secreted and isolated from a mixture of cells and medium containing the polypeptides. Alternatively, the polypeptide may be retained in the cytoplasm or in a membrane fraction and the cells harvested, lysed, and the polypeptide isolated using a desired method.

### XII. Examples

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1. Generation of TfR-Binding Polypeptides

Fc polypeptides that bind to TfR were engineered using combinatorial libraries as specific positions in the CH3 region, and selecting these libraries for binders to human TfR. Affinity maturation of initial TfR binding sequences led to identification of specific TfR-binding Fc polypeptides, for example, Fc polypeptide having the sequence of SEQ ID NO:66. An Fc polypeptide dimer-Fab fusion containing a heterodimeric Fc polypeptide dimer was constructed by co-expressing the following three polypeptides in a 1:1:2 ratio, respectively. The resulting tetrameric Fc polypeptide dimer-Fab fusion protein is termed BACE1-3C.35.21.

(1) Heavy chain containing a Fab region, a hinge region, and a modified Fc polypeptide fused to each other in this order in tandem series. The Fab region contains the heavy chain variable region of a BACE1-binding antibody. The hinge region has the sequence of SEQ ID NO:62. The Fc polypeptide has the sequence of SEQ ID NO:250 and contains the “knob” mutation (T366W according to EU numbering scheme) and the TfR-binding mutations.

(2) Heavy chain containing a Fab region, a hinge region, and a modified Fc polypeptide fused to each other in this order in tandem series. The Fab region contains the heavy chain variable region of a BACE1-binding antibody. The hinge region has the sequence of SEQ ID NO:62. The Fc polypeptide has the sequence of SEQ ID NO:251 and contains “hole” mutations (T366S, L368A, and Y407V according to EU numbering scheme).

(3) Light chain containing the light chain variable region of a BACE1-binding antibody.

DNA encoded for genes to express the three polypeptides above was cloned into expression vectors and transfected into ExpiCHO cells (Thermo Fisher Scientific) at a ratio of 1:1:2, respectively. After 5-7 days, the cells were harvested, and the resulting polypeptide was purified by Protein A followed by HIC using methods familiar to one with skill in the art. The resulting polypeptide was analyzed by mass spectrometry to demonstrate that no fusion proteins having homodimeric Fc polypeptide dimers (i.e., Fc polypeptide dimer having two Fc polypeptides both having the “knob” mutation, or Fc polypeptide dimer having two Fc polypeptides both having “hole” mutations) were present after purification. Additional tetramer polypeptides were generated analogously.

### Example 2. Generation of Human Apical Domain Knock-in Mice (Human TfR Knock-In (TfR^{ms/hu} KI) Mice)

Methods for generating knock-in/knock-out mice have been published in the literature and are well known to those with skill in the art. In summary, TfR^{ms/hu} KI mice were generated using CRISPR/Cas9 technology to express human Tfrc apical domain within the murine Tfrc gene; the resulting chimeric TfR was expressed in vivo under the control of the endogenous promoter. As described in International Patent Application No. PCT/US2018/018302, which is incorporated by reference in its entirety herein, C57B16 mice were used to generate a knock-in of the human apical TfR mouse line via pronuclear microinjection into single cell embryos, followed by embryo transfer to pseudo pregnant females. Specifically, Cas9, single guide RNAs having the sequences of SEQ ID NOS:264 and 265, and a donor DNA having the sequence of SEQ ID NO:267, were introduced into the embryos. The donor DNA comprised the human apical domain coding sequence (SEQ ID NO:266 that has been codon optimized for expression in mouse). The apical domain coding sequence was flanked with a left (nucleotides 1-817 of SEQ ID NO:267) and right homology arm (nucleotides 1523-2329 of SEQ ID NO:267). The donor sequence was designed in this manner such that the apical domain was to be inserted after the fourth mouse exon, and was immediately flanked at the 3′ end by the ninth mouse exon. A founder male from the progeny of the female that received the embryos was bred to wild-type females to generate F1 heterozygous mice. Homozygous mice were subsequently generated from breeding of F1 generation heterozygous mice.

### Example 3. TfR-Binding Fc Polypeptides Having LALA Mutations in Both Fc Polypeptides Prevent Reticulocyte Depletion in Mice

Antibodies that bind to TfR have been shown to deplete both circulating reticulocytes and bone marrow reticulocytes when administered to mice (see, e.g., Couch et al., Sci Transl Med, 5:183ra57, 1-12, 2013). An Fc polypeptide dimer-Fab fusion analogous to BACE1-3C.35.21 described in Example 1 was generated. The Fc polypeptide dimer-Fab fusion, termed “BACE1-3C.35.21^{2XLALA},” contains the following: (1) heavy chain containing a Fab region having heavy chain variable region of a BACE1-binding antibody, a hinge region, and a modified Fc polypeptide having the “knob” mutation (T366W according to EU numbering scheme), the mutations that reduce effector function (L234A and L235A according to EU numbering scheme), and the TfR-binding mutations (an anti-BACE1 Fab region fused to hinge region (SEQ ID NO:62) and SEQ ID NO:252 (clone CH3C.35.21 with knob and LALA mutations)); (2) heavy chain containing a Fab region having heavy chain variable region of a BACE1-binding antibody, a hinge region, and a modified Fc polypeptide having the “hole” mutations (T366S, L368A, and Y407V according to EU numbering scheme) and the mutations that reduce effector function (L234A and L235A according to EU numbering scheme) (an anti-BACE1 Fab region fused to hinge region (SEQ ID NO:62) and SEQ ID NO:253 (human Fc sequence with hole mutations and LALA mutations)); and (3) two light chain each comprising the light chain variable region of a BACE1-binding antibody.

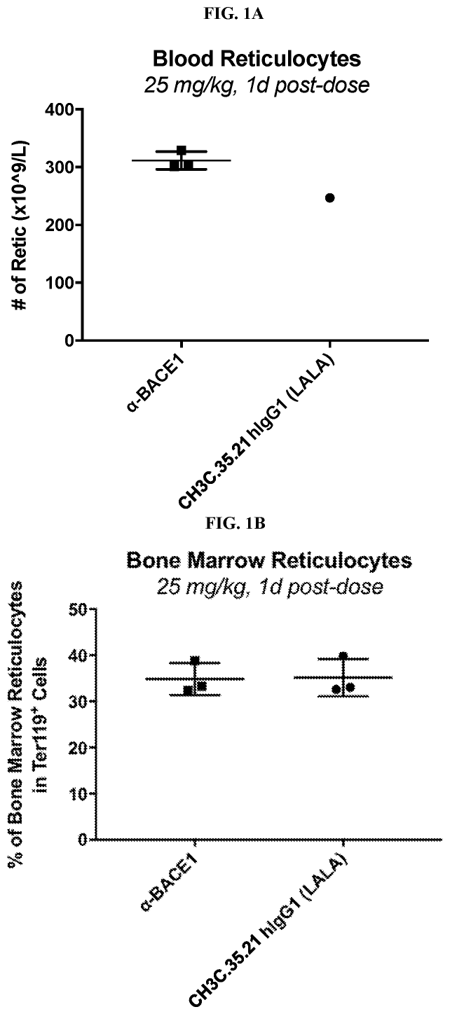

Homozygous human TfR knock-in (TfR^{ms/hu} KI) mice were generated to evaluate the Fc polypeptide dimer-Fab fusion proteins in vivo. Briefly, these mice were engineered to replace the mouse TfR with human apical domain/mouse chimeric TfR protein (see Example 2). These mice were dosed with BACE1-Fc dimer^{2XLALAPG} (anti-BACE1 Fab fused to an Fc dimer having LALA mutations and P329G mutation (according to EU numbering scheme) in both Fc polypeptides) or BACE1-3C.35.21^{2XLALA} at 25 mg/kg. Circulating and bone marrow reticulocytes were evaluated 24 hours post-dose by CBC and FACS analysis, respectively. Bone marrow reticulocytes were identified as the Ter119⁺, hCD71^{hi}, and FSC^{low} population. Consistent with previous studies, the Fc polypeptide dimer-Fab fusion, termed “BACE1-3C.35.21^{2XLALA},” did not induce reticulocyte depletion in vivo (FIGS. 1A and 1B), similar to a non-TfR binding Fc polypeptide dimer.

### Example 4. Design of TfR-Binding Fc Polypeptides Having Asymmetric LALA Mutations

TfR is highly expressed on reticulocytes, which are immature red blood cells present both in bone marrow and in circulation. It has previously been shown that TfR antibodies with full effector function can rapidly deplete reticulocytes in both blood and bone marrow. Thus, the depletion of reticulocytes is a major safety liability for TfR-based antibody therapeutics. However, complete removal of effector function in TfR-based antibodies is not an adequate solution because in some cases, it would be desirable to have effector function triggered upon Fab binding, but not upon TfR binding with an engineered TfR-binding Fc region fused to a therapeutic Fab. Because Fc mutations such as L234A and L235A (LALA) according to EU numbering scheme reduce FcγR binding to the Fc polypeptide dimer, engineered TfR-binding Fc polypeptide dimers fused to Fabs containing such mutations on both Fc polypeptides of the Fc polypeptide dimer are unable to elicit any effector function, with either TfR binding or Fab bound to target.

An engineered TfR-binding Fc polypeptide dimer fused to a therapeutic Fab that could elicit effector function when the Fab is bound to its target, but not when the TfR-binding site is bound to TfR, is desirable. To this end, Fc polypeptide dimers in which one of the two Fc polypeptides (but not the other) containing mutations that reduce FcγR binding when bound to TfR were developed. A series of TfR-binding Fc polypeptide dimers containing LALA mutations in one, both, or neither Fc polypeptides, fused to antigen-binding Fab regions that bind to either BACE1, human CD20 (hCD20), or mouse CD20 (mCD20), was generated as described in Tables 1 and 2 below. Table 1 describes the mutations in the Fc region of each of the two heavy chains. In all the variants described in Table 1, heavy chain 1 contains the TfR-binding site and the knob mutation T366W (according to EU numbering scheme) in the Fc region, and heavy chain 2 contains the hole mutations T366S, L368A, and Y407V (according to EU numbering scheme) in the Fc region. Variant zz-3C.35.21 does not contain any additional mutations in the Fc regions; variant zz-3C.35.21^{2XLALA} contains LALA mutations in both Fc regions of heavy chains 1 and 2; variant zz-3C.35.21^{cisLALA} contains LALA mutations in the same Fc region as the one that also contains the TfR-binding site (cisLALA or cis configuration); variant zz-3C.35.21^{ransLALA} contains LALA mutations in the Fc region that does not contain the TfR-binding site (transLALA or trans configuration). The same applies to variant zz-3C.35.23, variant zz-3C.35.23^{2XLALA}, variant zz-3C.35.23^{cisLALA}, and variant zz-3C.35.23^{transLALA}.

Table 2 lists the SEQ ID NO for each heavy chain and light chain of each Fc polypeptide dimer-Fab fusion. For example, BACE1-3C.35.21^{2XLALA} contains the variant zz-3C.35.21^{2XLALA} described in Table 1 and the Fab region that targets BACE1.

TABLE 1
Effector function mutations in Fc polypeptides

| | Heavy chain 1: | |
|---|---|---|
| | TfR-binding Fc | Heavy chain 2: |
| Variant name | (knob) | Fc (hole) |
| zz-3C.35.21 | None | None |
| zz-3C.35.21^{2xLALA} | LALA | LALA |
| zz-3C.35.21^{cisLALA} | LALA | None |
| zz-3C.35.21^{transLALA} | None | LALA |
| zz-3C.35.23 | None | None |
| zz-3C.35.23^{2xLALA} | LALA | LALA |
| zz-3C.35.23^{cisLALA} | LALA | None |
| zz-3C.35.23^{transLALA} | None | LALA |

TABLE 2
SEQ ID NOS for Fc polypeptide dimer-Fab fusions

| | Heavy chain 1 | Light chain 1 | Heavy chain 2 | Light chain 2 |
|---|---|---|---|---|
| BACE1-3C.35.21 | an anti-BACE1 | light chain | an anti-BACE1 | light chain |
| | Fab region | variable region | Fab region | variable region |
| | fused to hinge | of a BACE1- | fused to hinge | of a BACE1- |
| | region (SEQ ID | binding | region (SEQ ID | binding |
| | NO: 62) and | antibody | NO: 62) and | antibody |
| | SEQ ID | | SEQ ID | |
| | NO: 250 | | NO: 251 | |
| BACE1-3C.35.21^{2xLALA} | an anti-BACE1 | light chain | an anti-BACE1 | light chain |
| | Fab region | variable region | Fab region | variable region |
| | fused to hinge | of a BACE1- | fused to hinge | of a BACE1- |
| | region (SEQ ID | binding | region (SEQ ID | binding |
| | NO: 62) and | antibody | NO: 62) and | antibody |
| | SEQ ID | | SEQ ID | |
| | NO: 252 | | NO: 253 | |
| BACE1-3C.35.21^{cisLALA} | an anti-BACE1 | light chain | an anti-BACE1 | light chain |
| | Fab region | variable region | Fab region | variable region |
| | fused to hinge | of a BACE1- | fused to hinge | of a BACE1- |
| | region (SEQ ID | binding | region (SEQ ID | binding |
| | NO: 62) and | antibody | NO: 62) and | antibody |
| | SEQ ID | | SEQ ID | |
| | NO: 252 | | NO: 251 | |
| BACE1-3C.35.21^{transLALA} | an anti-BACE1 | light chain | an anti-BACE1 | light chain |
| | Fab region | variable region | Fab region | variable region |
| | fused to hinge | of a BACE1- | fused to hinge | of a BACE1- |
| | region (SEQ ID | binding | region (SEQ ID | binding |
| | NO: 62) and | antibody | NO: 62) and | antibody |
| | SEQ ID | | SEQ ID | |
| | NO: 250 | | NO: 253 | |
| hCD20-3C.35.21 | SEQ ID | SEQ ID | SEQ ID | SEQ ID |
| | NO: 254 | NO: 258 | NO: 256 | NO: 258 |
| hCD20-3C.35.21^{2xLALA} | SEQ ID | SEQ ID | SEQ ID | SEQ ID |
| | NO: NO: 255 | NO: 258 | NO: 257 | NO: 258 |
| hCD20-3C.35.21^{cisLALA} | SEQ ID | SEQ ID | SEQ ID | SEQ ID |
| | NO: NO: 255 | NO: 258 | NO: 257 | NO: 258 |
| hCD20-3C.35.21^{transLALA} | SEQ ID | SEQ ID | SEQ ID | SEQ ID |
| | NO: 254 | NO: 258 | NO: 256 | NO: 258 |
| mCD20-3C.35.21 | SEQ ID | SEQ ID | SEQ ID | SEQ ID |
| | NO: 259 | NO: 263 | NO: 261 | NO: 263 |
| mCD20-3C.35.21^{2xLALA} | SEQ ID | SEQ ID | SEQ ID | SEQ ID |
| | NO: 260 | NO: 263 | NO: 262 | NO: 263 |
| mCD20-3C.35.21^{cisLALA} | SEQ ID | SEQ ID | SEQ ID | SEQ ID |
| | NO: 260 | NO: 263 | NO: 261 | NO: 263 |
| BACE1-3C.35.23 | an anti-BACE1 | light chain | an anti-BACE1 | light chain |
| | Fab region | variable region | Fab region | variable region |
| | fused to hinge | of a BACE1- | fused to hinge | of a BACE1- |
| | region (SEQ ID | binding | region (SEQ ID | binding |
| | NO: 62) and | antibody | NO: 62) and | antibody |
| | SEQ ID | | SEQ ID | |
| | NO: 237 | | NO: 251 | |
| BACE1-3C.35.23^{2xLALA} | an anti-BACE1 | light chain | an anti-BACE1 | light chain |
| | Fab region | variable region | Fab region | variable region |
| | fused to hinge | of a BACE1- | fused to hinge | of a BACE1- |
| | region (SEQ ID | binding | region (SEQ ID | binding |
| | NO: 62) and | antibody | NO: 62) and | antibody |
| | SEQ ID | | SEQ ID | |
| | NO: 238 | | NO: 253 | |
| BACE1-3C.35.23^{cisLALA} | an anti-BACE1 | light chain | an anti-BACE1 | light chain |
| | Fab region | variable region | Fab region | variable region |
| | fused to hinge | of a BACE1- | fused to hinge | of a BACE1- |
| | region (SEQ ID | binding | region (SEQ ID | binding |
| | NO: 62) and | antibody | NO: 62) and | antibody |
| | SEQ ID | | SEQ ID | |
| | NO: 238 | | NO: 251 | |
| BACE1-3C.35.23^{transLALA} | an anti-BACE1 | light chain | an anti-BACE1 | light chain |
| | Fab region | variable region | Fab region | variable region |
| | fused to hinge | of a BACE1- | fused to hinge | of a BACE1- |
| | region (SEQ ID | binding | region (SEQ ID | binding |
| | NO: 62) and | antibody | NO: 62) and | antibody |
| | SEQ ID | | SEQ ID | |
| | NO: 237 | | NO: 253 | |
| hCD20-3C.35.23 | SEQ ID | SEQ ID | SEQ ID | SEQ ID |
| | NO: 410 | NO: 258 | NO: 256 | NO: 258 |
| hCD20-3C.35.23^{2xLALA} | SEQ ID | SEQ ID | SEQ ID | SEQ ID |
| | NO: NO: 411 | NO: 258 | NO: 257 | NO: 258 |
| hCD20-3C.35.23^{cisLALA} | SEQ ID | SEQ ID | SEQ ID | SEQ ID |
| | NO: NO: 411 | NO: 258 | NO: 257 | NO: 258 |
| hCD20-3C.35.23^{transLALA} | SEQ ID | SEQ ID | SEQ ID | SEQ ID |
| | NO: 410 | NO: 258 | NO: 256 | NO: 258 |
| mCD20-3C.35.23 | SEQ ID | SEQ ID | SEQ ID | SEQ ID |
| | NO: 412 | NO: 263 | NO: 261 | NO: 263 |
| mCD20-3C.35.23^{2xLALA} | SEQ ID | SEQ ID | SEQ ID | SEQ ID |
| | NO: 413 | NO: 263 | NO: 262 | NO: 263 |
| mCD20-3C.35.23^{cisLALA} | SEQ ID | SEQ ID | SEQ ID | SEQ ID |
| | NO: 413 | NO: 263 | NO: 261 | NO: 263 |

### Example 5. TfR-Binding Polypeptides Having Cis Configuration Attenuate Reticulocyte Depletion in Mice

To determine whether an Fc polypeptide dimer having the cis configuration or the trans configuration could attenuate reticulocyte depletion, we treated homozygous human TfR knock-in (TfR^{ms/hu} KI) mice with these Fc polypeptide dimers, as well as the wild-type human IgG (hIgG) counterpart, and analyzed reticulocytes from both the peripheral blood and the bone marrow in these animals. Circulating reticulocytes were quantified from peripheral blood using the Advia 120 Hematology System. Briefly, cells were stained with the ADVIA autoRETIC reagent and reticulocytes were identified based on RNA content and differential light absorption. For bone marrow reticulocytes, bone marrow cells were harvested from the femur of each animal, blocked with Fc-blocker (anti-mouse CD16/CD32), stained with anti-mTer119 and anti-hCD71, and analyzed by fluorescence-activated cell sorting (FACS). Reticulocytes were gated as the mTer119⁺, hCD71^{hi}, and FSC^{low} populations using the FlowJo analysis software. Upon analysis, the Fc polypeptide dimer having the cis configuration (i.e., only one Fc polypeptide contains both the TfR-binding site and the LALA mutations, while the other Fc polypeptide contains neither the TfR-binding site nor the LALA mutations) mitigated reticulocyte loss in both blood and bone marrow that was otherwise seen in the wild-type hIgG control and the Fc polypeptide dimer having the trans configuration (i.e., one Fc polypeptide contains the TfR-binding site and the other Fc polypeptide contains the LALA mutations). Remarkably, the Fc polypeptide dimer with the cis configuration at 25 mg/kg does not possess the major safety liability that is normally seen in TfR-binding polypeptides with effector function (FIGS. 2A and 2B). Furthermore, the system was stressed with a higher dose at 50 mg/kg, using a TfR-binding polypeptide with lower TfR affinity. Although the cis configuration partially attenuated blood reticulocyte loss, bone marrow reticulocyte loss, which is more reflective of clinical safety, was spared (FIGS. 2C and 2D). On the other hand, the Fc polypeptide dimer having the trans configuration depleted blood and bone marrow reticulocytes in a similar magnitude as the wild-type IgG control. These results demonstrate that the Fc polypeptide dimer with the cis configuration is able to mitigate reticulocyte loss in vivo.

### Example 6. TfR-Binding Polypeptides Having Cis Configuration Attenuate TfR-Mediated ADCC Activity In Vitro

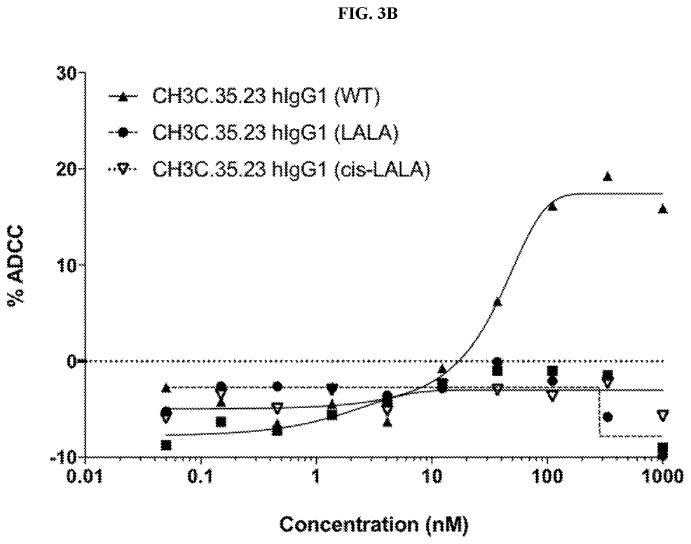

It was determined that the lack of reticulocyte loss in vivo by the Fc polypeptide dimer having the cis configuration is due to its inability to elicit TfR-mediated ADCC. Ramos cells, which express high levels of human TfR, were used as the target cells in an in vitro ADCC assay. The target cells were plated at 10,000 cells/well and opsonized with (1) hIgG1 with TfR-binding site, (2) hIgG1 with TfR-binding site and with LALA mutations in both Fc polypeptides, and (3) hIgG1 with an Fc polypeptide dimer having the cis configuration for 30 minutes. Effector natural killer (NK) cells were isolated from human peripheral blood, incubated overnight with IL-21 (20 ng/mL), and incubated with target cells at 25:1 effector:target cells ratio (250,000 cells/well) for 4 h. Cytotoxicity was evaluated by LDH expression, normalized to the control without any polypeptides, and calculated as the percentage of maximum lysis in target cells. Because effector immune cells, in this case natural killer cells, express FcγRs, the Fc portion of a wild-type IgG1 would bind to FcγRs and trigger an ADCC response. Indeed, the hIgG1 with TfR-binding site elicited a robust ADCC response, while both the hIgG1 with LALA mutations in both Fc polypeptides, and the hIgG1 with an Fc polypeptide dimer having the cis configuration did not. This data is consistent with the in vivo reticulocytes data: Fc polypeptide dimer having the cis configuration and Fc polypeptide dimer having the TfR-binding site and LALA mutations on both Fc polypeptides (at two different TfR affinities: FIG. 3A: CH3C.35.21; FIG. 3B: CH3C.35.23) prevent TfR-mediated ADCC and hence mitigated reticulocyte depletion.

### Example 7. TfR-Binding Polypeptides Prevent TfR-Mediated In Vitro CDC Activity

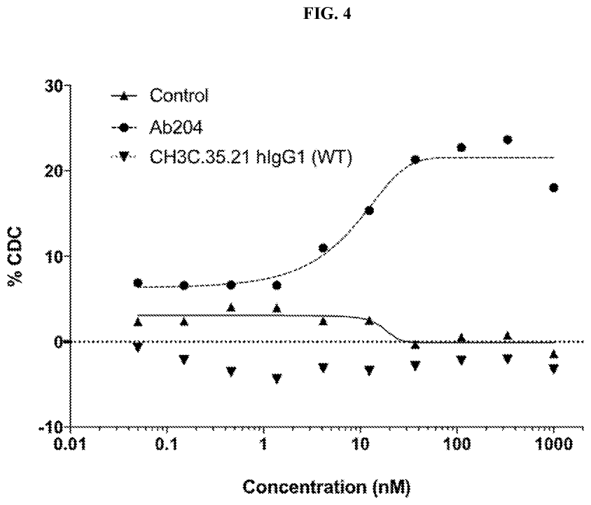

Reticulocyte loss in vivo by antibodies that bind to TfR could also be contributed by TfR-mediated CDC. Interestingly, the Fc polypeptide dimer that binds to TfR is unable to elicit CDC, likely due to its inability to sterically form polypeptide hexamers to trigger a complement response. CHO cells that were engineered to overexpress TfR (CHO-hTfR) were plated at 200,000 cells/well in serum-free media. The cells were opsonized with (1) control hIgG, (2) Ab204 (an anti-TfR positive control antibody), and (3) hIgG1 with TfR-binding site for 30 minutes. 50 μl it of diluted baby rabbit serum was added to each well and cells were incubated for 4 h. Cytotoxicity was evaluated by LDH expression, normalized to the control without any polypeptides, and calculated as the percentage of maximum lysis in target cells. Indeed, while anti-TfR Ab204 was able to induce CDC in CHO-hTfR cells, hIgG1 with TfR-binding site at the Fc region had no effect on CDC (FIG. 4).

### Example 8. TfR-Binding Polypeptides Having Cis Configuration Stimulate pSyk Activity in Primary Human Microglia

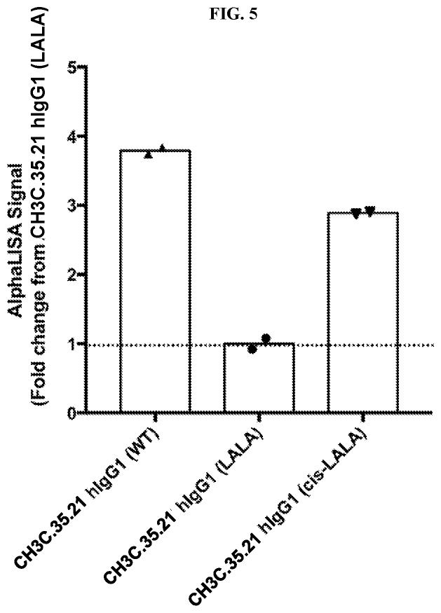

It was confirmed that the modified Fc polypeptide dimer has a functional Fc as determined by FcγR-induced phosphorylated-spleen tyrosine kinase (pSYK) activity, using primary human microglial cells. FcγRs on effector immune cells normally bind to the Fc region of an antibody to trigger a number of responses important for innate immunity. One of those responses include the Syk tyrosine kinase signaling pathway, which plays a critical role in immune cell activation such as phagocytosis, cytokine release, and ADCC (DeFranco et al., J. of Exp Med., 1997, 186(7):1027-39). When FcγRs of an immune cell bind to an immune complex, the immunoreceptor tyrosine-based activation motif (ITAM) enables Syk kinase to be recruited and phosphorylated by the Src family kinases, which results in downstream signaling pathways that trigger immune cell activation (Hirose et al., J of Biol Chem, 2004, 279:32308-15). Thus, pSyk is used as a readout for FcγR-induced immune cell activation. Microglial cells from a mixed glial culture derived from human fetal tissue were harvested and were used to evaluate pSyk activity in human microglial cells upon binding to TfR-binding polypeptides. Microglia were then added onto a TfR-binding polypeptides-coated 96-well plate, incubated at 37° C. for 10 min, lysed, and quantified for the levels of pSyk using a pSyk sandwich immunoassay. Even though the cis-LALA mutation did not elicit TfR-mediated ADCC, it was able to elicit a pSyk response in human microglial cells similar to the wild-type IgG peptide (about 3 fold increase from the LALA mutation control, FIG. 5). This result demonstrates that the modified Fc polypeptide dimer having the cis configuration retains its Fc function and could have effector function.

### Example 9. TfR-Binding Polypeptides Having Cis Configuration Elicit Fab-Mediated ADCC and CDC in Target Cells

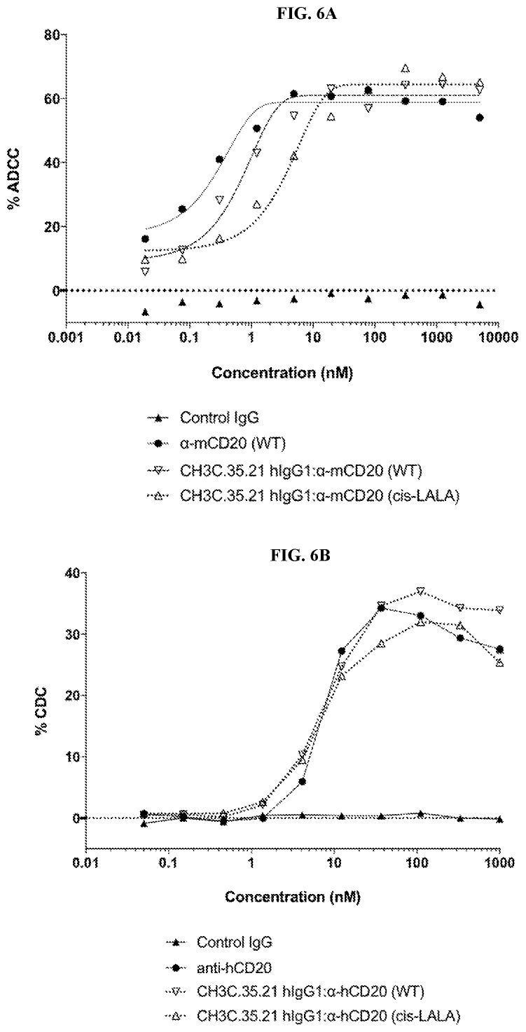

In addition to pSYK activity induction, the ability of TfR-binding polypeptides with the cis configuration to elicit Fab-mediated ADCC and CDC was evaluated. Target cells that express mouse CD20 (A20 cell line) were used to evaluate Fab-mediated ADCC. Similar to TfR-mediated ADCC described in Example 8, target cells were plated at 10,000 cells/well, opsonized, incubated with NK cells at 25:1 effector:target cells ratio, and evaluated for cytotoxicity by LDH expression. Target cells were opsonized with (1) control hIgG, (2) anti-mCD20 antibody, (3) hIgG1 with TfR-binding site and mCD20 Fab binding site (CH3C.35.21 hIgG1:α-mCD20 (WT)), and (4) hIgG1 with an Fc polypeptide dimer having the cis configuration and mCD20 Fab binding site (CH3C.35.21 hIgG1:α-mCD20 (cis-LALA)). The assay is designed to evaluate solely Fab binding since the target cells do not express human TfR. Consistent with pSYK activity induction, hIgG1 with an Fc polypeptide dimer having the cis configuration and mCD20 Fab binding site elicited ADCC similar to anti-mCD20 antibody and hIgG1 with TfR-binding site and mCD20 Fab binding site (FIG. 6A).

In addition to ADCC, CDC was also evaluated. Raji cells have previously been shown to be sensitive in anti-hCD20-mediated CDC; therefore, they were used as target cells to evaluate hIgG1 with TfR-binding site and hCD20 Fab binding site. Raji cells are plated at 200,000 cells/well in serum-free media and opsonized with (1) control hIgG, (2) anti-hCD20 antibody, (3) hIgG1 with TfR-binding site and hCD20 Fab binding site (CH3C.35.21 hIgG1:α-hCD20 (WT)), and (4) hIgG1 with an Fc polypeptide dimer having the cis configuration and hCD20 Fab binding site (CH3C.35.21 hIgG1:α-hCD20 (cis-LALA)) for 30 minutes. 50 μL of diluted baby rabbit serum was added to each well and cells were incubated for 4 h. Cytotoxicity was evaluated by LDH expression, normalized to the control without any polypeptides, and calculated as the percentage of maximum lysis in target cells. Similar to Fab-mediated ADCC, hIgG1 with an Fc polypeptide dimer having the cis configuration and hCD20 Fab binding site elicited CDC to the same degree as anti-hCD20 and hIgG1 with TfR-binding site and hCD20 Fab binding site (FIG. 6B). Taken together, these functional in vitro cytotoxicity assays demonstrate that hIgG1 with an Fc polypeptide dimer having the cis configuration does not interfere with its ability to elicit Fab-mediated effector function.

### Example 10. TfR-Binding Polypeptides Having Cis Configuration and mCD20 Fab Binding Site Elicit Effector Function In Vivo

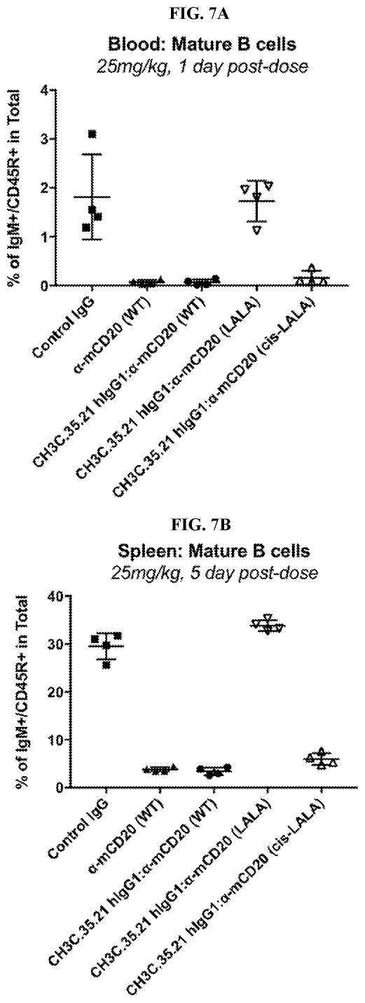

As demonstrated in in vivo safety analysis, hIgG1 with an Fc polypeptide dimer having the cis configuration mitigated reticulocyte loss upon binding to TfR. We then tested whether this configuration could elicit Fab-mediated effector function in vivo, which is essential to induce a therapeutic response. Antibodies against mCD20 have previously been demonstrated to severely deplete peripheral and splenic B cells in vivo and have therefore been utilized to evaluate Fab-mediated effector function. The ability of hIgG1 with an Fc polypeptide dimer having the cis configuration and mCD20 Fab binding site to deplete blood and splenic B cells was evaluated in WT mice, and compared to the response observed in anti-mCD20 antibody and hIgG1 with TfR-binding site and mCD20 Fab binding site. WT mice were treated at 25 mg/kg with (1) control IgG, (2) anti-mCD20 antibody, (3) hIgG1 with TfR-binding site and mCD20 Fab binding site (CH3C.35.21 hIgG1:α-mCD20 (WT)), (4) hIgG1 with TfR-binding site and with LALA mutations in both Fc polypeptides and mCD20 Fab binding site (CH3C.35.21 hIgG1:α-mCD20 (LALA)), and (5) hIgG1 with an Fc polypeptide dimer having the cis configuration and mCD20 Fab binding site (CH3C.35.21 hIgG1:α-mCD20 (cis-LALA)). Mature peripheral B cells and splenic B cells were evaluated on Day 1 and Day 5, respectively. Briefly, peripheral blood and spleen were harvested. Cells from peripheral blood and splenocytes were treated with ACK lysis buffer, incubated with Fc blocker, and stained with anti-B220 and anti-IgM. Mature B cells were identified as the B220^{high}IgM^{high} population upon FACS analysis. Consistent with Fab-mediated in vitro ADCC and CDC assays, hIgG1 with an Fc polypeptide dimer having the cis configuration and mCD20 Fab binding site elicited robust B cell depletion similar to the anti-mCD20 antibody and hIgG1 with TfR-binding site and mCD20 Fab binding site (FIGS. 7A and 7B). These results demonstrate that the modified Fc polypeptide dimer having the cis configuration retains its Fc function and has Fab-mediated effector function in vivo.

### Example 11. Modified Fc Polypeptides that Bind to TfR

This example describes modifications to Fc polypeptides to confer TfR binding and transport across the BBB.

Unless otherwise indicated, the positions of amino acid residues in this section are numbered based on EU index numbering for a human IgG1 wild-type Fc region.

### Generation and Characterization of Fc Polypeptides Comprising Modifications at Positions 384, 386, 387, 388, 389, 390, 413, 416, and 421 (CH3C Clones)

Yeast libraries containing Fc regions having modifications introduced into positions including amino acid positions 384, 386, 387, 388, 389, 390, 413, 416, and 421 were generated as described below. Illustrative clones that bind to TfR are shown in Tables 3 and 4.

After an additional two rounds of sorting, single clones were sequenced and four unique sequences were identified. These sequences had a conserved Trp at position 388, and all had an aromatic residue (i.e., Trp, Tyr, or His) at position 421. There was a great deal of diversity at other positions.

The four clones selected from the library were expressed as Fc fusions to Fab fragments in CHO or 293 cells, and purified by Protein A and size-exclusion chromatography, and then screened for binding to human TfR in the presence or absence of holo-Tf by ELISA. The clones all bound to human TfR and the binding was not affected by the addition of excess (5 μM) holo-Tf. Clones were also tested for binding to 293F cells, which endogenously express human TfR. The clones bound to 293F cells, although the overall binding was substantially weaker than the high-affinity positive control.

Next, it was tested whether clones could internalize in TfR-expressing cells using clone CH3C.3 as a test clone. Adherent HEK 293 cells were grown in 96-well plates to about 80% confluence, media was removed, and samples were added at 1 μM concentrations: clone CH3C.3, anti-TfR benchmark positive control antibody (Ab204), anti-BACE1 benchmark negative control antibody (Ab107), and human IgG isotype control (obtained from Jackson Immunoresearch). The cells were incubated at 37° C. and 8% CO₂ concentration for 30 minutes, then washed, permeabilized with 0.1% Triton™ X-100, and stained with anti-human-IgG-Alexa Fluor® 488 secondary antibody. After additional washing, the cells were imaged under a high content fluorescence microscope (i.e., an Opera Phenix™ system), and the number of puncta per cell was quantified. At 1 μM, clone CH3C.3 showed a similar propensity for internalization to the positive anti-TfR control, while the negative controls showed no internalization.

### Further Engineering of Clones

Additional libraries were generated to improve the affinity of the initial hits against human TfR using a soft randomization approach, wherein DNA oligos were generated to introduce soft mutagenesis based on each of the original four hits. Additional clones were identified that bound TfR and were selected. The selected clones fell into two general sequence groups. Group 1 clones (i.e., clones CH3C.18, CH3C.21, CH3C.25, and CH3C.34) had a semi-conserved Leu at position 384, a Leu or His at position 386, a conserved and a semi-conserved Val at positions 387 and 389, respectively, and a semi-conserved P-T-W motif at positions 413, 416, and 421, respectively. Group 2 clones had a conserved Tyr at position 384, the motif TXWSX at positions 386-390, and the conserved motif S/T-E-F at positions 413, 416, and 421, respectively. Clones CH3C.18 and CH3C.35 were used in additional studies as representative members of each sequence group.

### Epitope Mapping

To determine whether the engineered Fc regions bound to the apical domain of TfR, TfR apical domain was expressed on the surface of phage. To properly fold and display the apical domain, one of the loops had to be truncated and the sequence needed to be circularly permuted. Clones CH3C.18 and CH3C.35 were coated on ELISA plates and a phage ELISA protocol was followed. Briefly, after washing and blocking with 1% PBSA, dilutions of phage displaying were added and incubated at room temperature for 1 hour. The plates were subsequently washed and anti-M13-HRP was added, and after additional washing the plates were developed with TMB substrate and quenched with 2N H₂SO₄. Both clones CH3C.18 and CH3C.35 bound to the apical domain in this assay.

### Paratope Mapping

To understand which residues in the Fc domain were most important for TfR binding, a series of mutant clone CH3C.18 and clone CH3C.35 Fc regions was created in which each mutant had a single position in the TfR binding register mutated back to wild-type. The resulting variants were expressed recombinantly as Fab-Fc fusions and tested for binding to human or cyno TfR. For clone CH3C.35, positions 388 and 421 were important for binding; reversion of either of these to wild-type completely ablated binding to human TfR.

### Binding Characterization of Maturation Clones

Binding ELISAs were conducted with purified Fab-Fc fusion variants with human or cyno TfR coated on the plate, as described above. The variants from the clone CH3C.18 maturation library, clone CH3C.3.2-1, clone CH3C.3.2-5, and clone CH3C.3.2-19, bound human and cyno TfR with approximately equivalent EC₅₀ values, whereas the parent clones CH3C.18 and CH3C.35 had greater than 10-fold better binding to human versus cyno TfR.

Next, it was tested whether the modified Fc polypeptides internalized in human and monkey cells. Using the protocol described above, internalization in human HEK 293 cells and rhesus LLC-MK2 cells was tested. The variants that similarly bound human and cyno TfR, clones CH3C.3.2-5 and CH3C.3.2-19, had significantly improved internalization in LLC-MK2 cells as compared with clone CH3C.35.

### Additional Engineering of Clones

Additional engineering to further affinity mature clones CH3C.18 and CH3C.35 involved adding additional mutations to the positions that enhanced binding through direct interactions, second-shell interactions, or structure stabilization. This was achieved via generation and selection from an “NNK walk” or “NNK patch” library. The NNK walk library involved making one-by-one NNK mutations of residues that are near to the paratope. By looking at the structure of Fc bound to FcγRI (PDB ID: 4W4O), 44 residues near the original modification positions were identified as candidates for interrogation. Specifically, the following residues were targeted for NNK mutagenesis: K248, R255, Q342, R344, E345, Q347, T359, K360, N361, Q362, S364, K370, E380, E382, S383, G385, Y391, K392, T393, D399, S400, D401, S403, K409, L410, T411, V412, K414, S415, Q418, Q419, G420, V422, F423, S424, S426, Q438, S440, S442, L443, S444, P4458, G446, and K447. The 44 single point NNK libraries were generated using Kunkel mutagenesis, and the products were pooled and introduced to yeast via electroporation, as described above for other yeast libraries.

The combination of these mini-libraries (each of which had one position mutated, resulting in 20 variants) generated a small library that was selected using yeast surface display for any positions that lead to higher affinity binding. Selections were performed as described above, using TfR apical domain proteins. After three rounds of sorting, clones from the enriched yeast library were sequenced, and several “hot-spot” positions were identified where certain point mutations significantly improved the binding to apical domain proteins. For clone CH3C.35, these mutations included E380 (mutated to Trp, Tyr, Leu, or Gln) and 5415 (mutated to Glu). The sequences of the clone CH3C.35 single and combination mutants are set forth in SEQ ID NOS:21-23, 64-69, and 125-127. For clone CH3C.18, these mutations included E380 (mutated to Trp, Tyr, or Leu) and K392 (mutated to Gln, Phe, or His). The sequences of the clone CH3C.18 single mutants are set forth in SEQ ID NOS:70-75.

### Additional Maturation Libraries to Improve Clone CH3C.35 Affinity

An additional library to identify combinations of mutations from the NNK walk library, while adding several additional positions on the periphery of these, was generated as described for previous yeast libraries. In this library, the YxTEWSS (SEQ ID NO:414) and TxxExxxxF (SEQ ID NO:415) motifs were kept constant, and six positions were completely randomized: E380, K392, K414, 5415, 5424, and 5426. Positions E380 and 5415 were included because they were “hot spots” in the NNK walk library. Positions K392, S424, and S426 were included because they make up part of the core that may position the binding region, while K414 was selected due to its adjacency to position 415.

This library was sorted, as previously described, with the cyno TfR apical domain only. The enriched pool was sequenced after five rounds, and the sequences of the modified regions of the identified unique clones are set forth in SEQ ID NOS:76-93.

The next libraries were designed to further explore acceptable diversity in the main binding paratope. Each of the original positions (384, 386, 387, 388, 389, 390, 413, 416, and 421) plus the two hot spots (380 and 415) were individually randomized with NNK codons to generate a series of single-position saturation mutagenesis libraries on yeast. In addition, each position was individually reverted to the wild-type residue, and these individual clones were displayed on yeast. It was noted that positions 380, 389, 390, and 415 were the only positions that retained substantial binding to TfR upon reversion to the wild-type residue (some residual but greatly diminished binding was observed for reversion of 413 to wild-type).

The single-position NNK libraries were sorted for three rounds against the human TfR apical domain to collect the top ˜5% of binders, and then at least 16 clones were sequenced from each library. The results indicate what amino acids at each position can be tolerated without significantly reducing binding to human TfR, in the context of clone CH3C.35. A summary is below:

### Position 380: Trp, Leu, or Glu;

### Position 384: Tyr or Phe;

Position 386: Thr only;
Position 387: Glu only;
Position 388: Trp only;
Position 389: Ser, Ala, or Val (although the wild type Asn residue seems to retain some binding, it did not appear following library sorting);

### Position 390: Ser or Asn;

### Position 413: Thr or Ser;

### Position 415: Glu or Ser;

Position 416: Glu only; and
Position 421: Phe only.

The above residues, when substituted into clone CH3C.35 as single changes or in combinations, represent paratope diversity that retains binding to TfR apical domain. Clones having mutations at these positions include those shown in Table 4, and the sequences of the CH3 domains of these clones are set forth in SEQ ID NOS:65-69, 92, 94-124, and 268-274.

### Example 12. Methods

### Generation of Phage-Display Libraries

A DNA template coding for the wild-type human Fc sequence was synthesized and incorporated into a phagemid vector. The phagemid vector contained an ompA or pelB leader sequence, the Fc insert fused to c-Myc and 6×His (SEQ ID NO:421) epitope tags, and an amber stop codon followed by M13 coat protein pIII.

Primers containing “NNK” tricodons at the desired positions for modifications were generated, where N is any DNA base (i.e., A, C, G, or T) and K is either G or T. Alternatively, primers for “soft” randomization were used, where a mix of bases corresponding to 70% wild-type base and 10% of each of the other three bases was used for each randomization position. Libraries were generated by performing PCR amplification of fragments of the Fc region corresponding to regions of randomization and then assembled using end primers containing SfiI restriction sites, then digested with SfiI and ligated into the phagemid vectors. Alternatively, the primers were used to conduct Kunkel mutagenesis. The ligated products or Kunkel products were transformed into electrocompetent E. coli cells of strain TG1 (obtained from Lucigen). The E. coli cells were infected with M13K07 helper phage after recovery and grown overnight, after which library phage were precipitated with 5% PEG/NaCl, resuspended in 15% glycerol in PBS, and frozen until use. Typical library sizes ranged from about 10⁹ to about 10¹¹ transformants. Fc-dimers were displayed on phage via pairing between pIII-fused Fc and soluble Fc not attached to pIII (the latter being generated due to the amber stop codon before pIII).

### Generation of Yeast-Display Libraries

A DNA template coding for the wild-type human Fc sequence was synthesized and incorporated into a yeast display vector. For CH2 and CH3 libraries, the Fc polypeptides were displayed on the Aga2p cell wall protein. Both vectors contained prepro leader peptides with a Kex2 cleavage sequence, and a c-Myc epitope tag fused to the terminus of the Fc.

Yeast display libraries were assembled using methods similar to those described for the phage libraries, except that amplification of fragments was performed with primers containing homologous ends for the vector. Freshly prepared electrocompetent yeast (i.e., strain EBY100) were electroporated with linearized vector and assembled library inserts. Electroporation methods will be known to one of skill in the art. After recovery in selective SD-CAA media, the yeast were grown to confluence and split twice, then induced for protein expression by transferring to SG-CAA media. Typical library sizes ranged from about 10⁷ to about 10⁹ transformants. Fc-dimers were formed by pairing of adjacently displayed Fc monomers.

### General Methods for Phage Selection

Phage methods were adapted from Phage Display: A Laboratory Manual (Barbas, 2001). Additional protocol details can be obtained from this reference.

### Plate Sorting Methods

Human TfR target was coated on MaxiSorp® microtiter plates (typically 200 μL at 1-10 μg/mL in PBS) overnight at 4° C. All binding was done at room temperature unless otherwise specified. The phage libraries were added into each well and incubated overnight for binding. Microtiter wells were washed extensively with PBS containing 0.05% Tween® 20 (PBST) and bound phage were eluted by incubating the wells with acid (typically 50 mM HCl with 500 mM KCl, or 100 mM glycine, pH 2.7) for 30 minutes. Eluted phage were neutralized with 1 M Tris (pH 8) and amplified using TG1 cells and M13/K07 helper phage and grown overnight at 37° C. in 2YT media containing 50 μg/mL carbenacillin and 50 μg/mL Kanamycin. The titers of phage eluted from a target-containing well were compared to titers of phage recovered from a non-target-containing well to assess enrichment. Selection stringency was increased by subsequently decreasing the incubation time during binding and increasing washing time and number of washes.

### Bead Sorting Methods

Antigen was biotinylated through free amines using NHS-PEG4-Biotin (obtained from Pierce™). For biotinylation reactions, a 3- to 5-fold molar excess of biotin reagent was used in PBS. Reactions were quenched with Tris followed by extensive dialysis in PBS. The biotinylated antigen was immobilized on streptavidin-coated magnetic beads, (i.e., M280-streptavidin beads obtained Thermo Fisher). The phage display libraries were incubated with the antigen-coated beads at room temperature for 1 hour. The unbound phage were then removed and beads were washed with PBST. The bound phage were eluted by incubating with 50 mM HCl containing 500 mM KCl (or 0.1 M glycine, pH 2.7) for 30 minutes, and then neutralized and propagated as described above for plate sorting.

After three to five rounds of panning, single clones were screened by either expressing Fc on phage or solubly in the E. coli periplasm. Such expression methods will be known to one of skill in the art. Individual phage supernatants or periplasmic extracts were exposed to blocked ELISA plates coated with antigen or a negative control and were subsequently detected using HRP-conjugated goat anti-Fc (obtained from Jackson Immunoresearch) for periplasmic extracts or anti-M13 (GE Healthcare) for phage, and then developed with TMB reagent (obtained from Thermo Fisher). Wells with OD₄₅₀ values greater than around 5-fold over background were considered positive clones and sequenced, after which some clones were expressed either as a soluble Fc fragment or fused to Fab fragments.

### General Methods for Yeast Selection

### Bead Sorting (Magnetic-Assisted Cell Sorting (MACS)) Methods

MACS and FACS selections were performed similarly to as described in Ackerman, et al. 2009 Biotechnol. Prog. 25(3), 774. Streptavidin magnetic beads (e.g., M-280 streptavidin beads from Thermo Fisher) were labeled with biotinylated antigen and incubated with yeast (typically 5-10× library diversity). Unbound yeast were removed, the beads were washed, and bound yeast were grown in selective media and induced for subsequent rounds of selection.

### Fluorescence-Activated Cell Sorting (FACS) Methods

Yeast were labeled with anti-c-Myc antibody to monitor expression and biotinylated antigen (concentration varied depending on the sorting round). In some experiments, the antigen was pre-mixed with streptavidin-Alexa Fluor® 647 in order to enhance the avidity of the interaction. In other experiments, the biotinylated antigen was detected after binding and washing with streptavidin-Alexa Fluor® 647. Singlet yeast with binding were sorted using a FACS Aria III cell sorter. The sorted yeast were grown in selective media then induced for subsequent selection rounds.

After an enriched yeast population was achieved, yeast were plated on SD-CAA agar plates and single colonies were grown and induced for expression, then labeled as described above to determine their propensity to bind to the target. Positive single clones were subsequently sequenced for binding antigen, after which some clones were expressed either as a soluble Fc fragment or as fused to Fab fragments.

### General Methods for Screening

### Screening by ELISA

Clones were selected from panning outputs and grown in individual wells of 96-well deep-well plates. The clones were either induced for periplasmic expression using autoinduction media (obtained from EMD Millipore) or infected with helper phage for phage-display of the individual Fc variants on phage. The cultures were grown overnight and spun to pellet E. coli. For phage ELISA, phage containing supernatant was used directly. For periplasmic expression, pellets were resuspended in 20% sucrose, followed by dilution at 4:1 with water, and shaken at 4° C. for 1 hour. Plates were spun to pellet the solids and supernatant was used in the ELISA.

ELISA plates were coated with antigen, typically at 0.5 mg/mL overnight, then blocked with 1% BSA before addition of phage or periplasmic extracts. After a 1-hour incubation and washing off unbound protein, HRP-conjugated secondary antibody was added (i.e., anti-Fc or anti-M13 for soluble Fc or phage-displayed Fc, respectively) and incubated for 30 minutes. The plates were washed again, and then developed with TMB reagent and quenched with 2N sulfuric acid. Absorbance at 450 nm was quantified using a plate reader (BioTek®) and binding curves were plotted using Prism software where applicable. Absorbance signal for tested clones was compared to negative control (phage or paraplasmic extract lacking Fc). In some assays, soluble transferrin or other competitor was added during the binding step, typically at significant molar excess (greater than 10-fold excess).

### Screening by Flow Cytometry

Fc variant polypeptides (expressed either on phage, in periplasmic extracts, or solubly as fusions to Fab fragments) were added to cells in 96-well V-bottom plates (about 100,000 cells per well in PBS+1% BSA (PBSA)), and incubated at 4° C. for 1 hour. The plates were subsequently spun and the media was removed, and then the cells were washed once with PBSA. The cells were resuspended in PBSA containing secondary antibody (typically goat anti-human-IgG-Alexa Fluor® 647 (obtained from Thermo Fisher)). After 30 minutes, the plates were spun and the media was removed, the cells were washed 1-2 times with PBSA, and then the plates were read on a flow cytometer (i.e., a FACSCanto™ II flow cytometer). Median fluorescence values were calculated for each condition using FlowJo software and binding curves were plotted with Prism software.

### Example 13. Construction of CH3C.18 Variants

This example describes the construction of a library of CH3C.18 variants.

Single clones were isolated, and grown overnight in SG-CAA media supplemented with 0.2% glucose overnight to induce surface expression of CH3C.18 variants. For each clone, two million cells were washed three times in PBS+0.5% BSA at pH 7.4. Cells were stained with biotinylated target, 250 nM human TfR, 250 nM cyno TfR, or 250 nM of an unrelated biotinylated protein for 1 hour at 4° C. with shaking, then washed twice with the same buffer. Cells were stained with nuetravidin-Alexafluor647 (AF647) for 30 minutes at 4° C., then washed twice again. Expression was measured using anti-c-myc antibody with anti-chicken-Alexfluor488 (AF488) secondary antibody. Cells were resuspended, and median fluorescence intensity (MFI) of AF647 and AF488 was measured on a BD FACS CantoII. MFI was calculated for the TfR-binding population for each population and plotted with human TfR, cyno TfR, or control binding.

Table 5 shows the library of CH3C.18 variants. Each row represents a variant that contains the indicated amino acid substitutions at each position and the amino acids at the rest of the positions are the same as those in CH3C.18. The positions shown in Table 5 are numbered according to the EU numbering scheme.

TABLE 5
CH3C.18 Variants

| Position | 384 | 386 | 387 | 389 | 390 | 391 | 413 | 416 | 421 |
|---|---|---|---|---|---|---|---|---|---|
| Wild-type Fc | N | Q | P | N | N | Y | D | R | N |
| CH3C.4 (CH3C.18.1) | V | T | P | A | L | Y | L | E | W |
| CH3C.2 (CH3C.18.2) | Y | T | V | S | H | Y | S | E | Y |
| CH3C.3 (CH3C.18.3) | Y | T | E | S | Q | Y | E | D | H |
| CH3C.1 (CH3C.18.4) | L | L | V | V | G | Y | A | T | W |
| CH3C.18 (CH3C.18.1.18) | L | H | V | A | V | Y | P | T | W |
| CH3C.3.1-3 (CH3C.18.3.1-3) | L | H | V | V | A | T | P | T | W |
| CH3C.3.1-9 (CH3C.18.3.1-9) | L | P | V | V | H | T | P | T | W |
| CH3C.3.2-1 (CH3C.18.3.2-1) | L | H | V | V | N | F | P | T | W |
| CH3C.3.2-5 (CH3C.18.3.2-5) | L | H | V | V | D | Q | P | T | W |
| CH3C.3.2-19 (CH3C.18.3.2- | L | H | V | V | N | Q | P | T | W |
| 19) | | | | | | | | | |
| CH3C.3.4-1 (CH3C.18.3.4-1) | W | F | V | S | T | T | P | N | F |
| CH3C.3.4-19 (CH3C.18.3.4- | W | H | V | S | T | T | P | N | Y |
| 19) | | | | | | | | | |
| CH3C.3.2-3 (CH3C.18.3.2-3) | L | H | V | V | E | Q | P | T | W |
| CH3C.3.2-14 (CH3C.18.3.2- | L | H | V | V | G | V | P | T | W |
| 14) | | | | | | | | | |
| CH3C.3.2-24 (CH3C.18.3.2- | L | H | V | V | H | T | P | T | W |
| 24) | | | | | | | | | |
| CH3C.3.4-26 (CH3C.18.3.4- | W | T | V | G | T | Y | P | N | Y |
| 26) | | | | | | | | | |
| CH3C.3.2-17 (CH3C.18.3.2- | L | H | V | V | G | T | P | T | W |
| 17) | | | | | | | | | |

### Example 14. TfR-Binding Polypeptides Having Cis Configuration and Fabs that Bind Amyloid Beta (Aβ) Effectively Cross the BBB and Elicit Robust Effector Function, Leading to Microglial Recruitment to Aβ Plaques and Plaque Reduction in an Aβ Plaque Mouse Model

To provide additional evidence that TfR-binding polypeptides having cis configuration could be used in a therapeutically relevant disease model, we evaluated TfR-binding Fc polypeptides having the cis configuration possessing Fab that bind Aβ in an Aβ plaque depositing mouse model. In particular, these polypeptides were evaluated for the ability to recruit microglia to Aβ plaques. Briefly, animals (3.5 months old) were treated at 50 mg/kg intraperitoneally on Days 0, 3, 6, and 9 in the following groups: 1) TfR^{ms/hu} KI mice treated with control IgG (n=6), 2) 5XFAD×TfR_{ms/hu} KI mice treated with control IgG (n=11), 3) 5XFAD×TfR^{ms/hu} KI mice treated with anti-Aβ (α-Aβ) (n=12), 4) 5XFAD×TfR^{ms/hu} KI mice treated with TfR-binding Fc polypeptides having the cis configuration and Aβ Fab binding site (n=12), and 5) 5XFAD×TfR^{ms/hu} KI mice treated with TfR-binding Fc polypeptides having LALA mutations on both Fc polypeptides (n=12). Table 6 lists the SEQ ID NO for each heavy chain and light chain of each Fc polypeptide dimer-Fab fusion.

TABLE 6
SEQ ID NOS for Fc polypeptide dimer-Fab fusions

| | Heavy chain 1 | Light chain 1 | Heavy chain 2 | Light chain 2 |
|---|---|---|---|---|
| anti-Aβ-Fc polypeptides | SEQ ID NO: 416 | SEQ ID NO: 420 | SEQ ID NO: 416 | SEQ ID NO: 420 |
| | (an anti-Aβ | (light chain | (an anti-Aβ | (light chain |
| | Fab region | of anti-Aβ | Fab region | of anti-Aβ |
| | fused to hinge | Fab region) | fused to hinge | Fab region) |
| | region and | | region and | |
| | wild-type human | | wild-type human | |
| | Fc polypeptide) | | Fc polypeptide) | |
| anti-Aβ-3C.35.23.4^{2xLALA} | SEQ ID NO: 417 | SEQ ID NO: 420 | SEQ ID NO:418 | SEQ ID NO: 420 |
| | (an anti-Aβ | (light chain | (an anti-Aβ | (light chain |
| | Fab region | of anti-Aβ | Fab region | of anti-Aβ |
| | fused to hinge | Fab region) | fused to hinge | Fab region) |
| | region and | | region and | |
| | 3C.35.23.4 with | | Fc with hole and | |
| | knob and LALA | | LALA mutations) | |
| | mutations) | | | |
| anti-Aβ-3C.35.23.4^{cisLALA} | SEQ ID NO: 417 | SEQ ID NO: 420 | SEQ ID NO: 419 | SEQ ID NO: 420 |
| | (an anti-Aβ | (light chain | (an anti-Aβ | (light chain |
| | Fab region | of anti-Aβ | Fab region | of anti-Aβ |
| | fused to hinge | Fab region) | fused to hinge | Fab region) |
| | region and | | region and | |
| | 3C.35.23.4 with | | Fc with hole | |
| | knob and LALA | | mutations) | |
| | mutations) | | | |

On Day 12, mice were perfused; brains were collected and sectioned sagittally at 40 μm for immunohistochemistry. Two brain sections (section 1: ˜3 mm lateral to the midline, section 2: at midline) per animal were selected for IHC analysis. Free-floating sections were incubated in blocking solution (5% donkey serum in PBS with 0.3% triton X-100) for two hours at room temperature, then with primary antibodies (anti-CD68, anti-human Abeta) overnight at 4° C. Sections were then washed 3× for 15 minutes, incubated with fluorescence-labelled secondary antibodies for 2 h at room temperature, DAPI solution for 20 minutes, and then washed 3× in PBS with 0.3% triton X-100. Sections were mounted onto slides and coverslipped using Prolong Glass Antifade mounting medium. Slides were imaged using a Zeiss Axioscan.Z1 slide scanner at 20× magnification and processed using custom macros in Zeiss ZEN software and image processing macros. Specifically, images were analyzed to determine the dilated plaque area (by size), area of plaque/CD68 microglia overlap, dilated CD68⁺ microglia area, count, and pixel intensity sum (by size: 9-14 μm², 14-33 μm², 33-75 μm², and 75-3,333 μm²), and plaque morphology, area, count, and pixel intensity sum (separating circular from irregular plaques using compactness <0.7 for irregular and >0.7 for circular; separating by size: 30-125 μm², 125-250 μm², 250-500 μm², and 500-3,300 μm²).

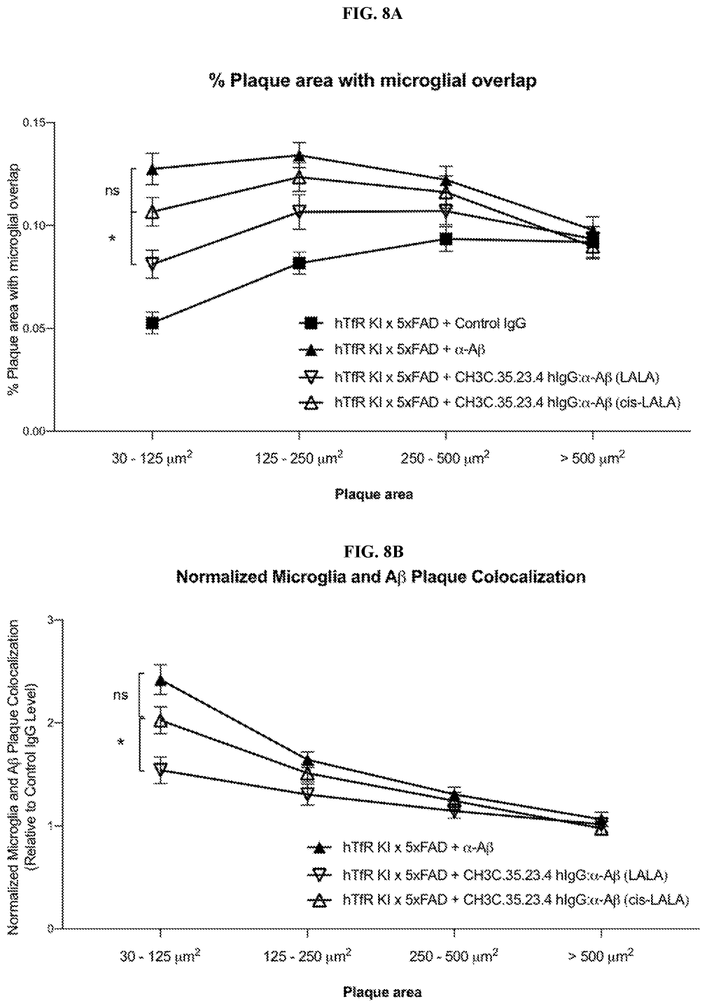

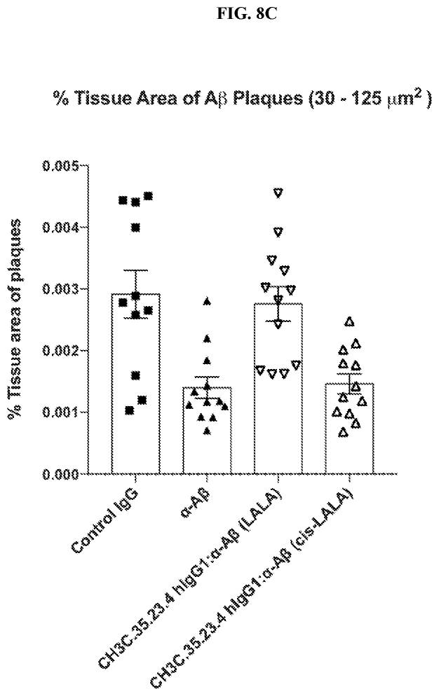

From these experiments, 5XFAD×TfR^{ms/hu} KI mice treated with anti-Aβ having a TfR-binding site with cis-LALA Fc polypeptide dimer elicited robust microglial recruitment towards Aβ plaques (as measured by colocalization of microglial marker CD68 and a Aβ marker) and reduced smaller plaques sized at 30-125 μm², in a manner similar to anti-Aβ (FIGS. 8A-8C). Importantly, these effects were not observed with anti-Aβ having a TfR-binding site with LALA mutations on both Fc polypeptides, which is consistent with the necessity of effector function in this disease paradigm. Overall, these data, along with other in vitro and in vivo data herein, provide compelling evidence that the platform backbone of TfR-binding Fc polypeptides with a cis-LALA configuration and relevant Fab binding site can mitigate reticulocyte safety, as well as elicit target-mediated effector function in a relevant disease model (i.e., microglial engagement in the brain).

It is understood that the examples and embodiments described herein are for illustrative purposes only and that various modifications or changes in light thereof will be suggested to persons skilled in the art and are to be included within the spirit and purview of this application and scope of the appended claims. The sequences of the sequence accession numbers cited herein are hereby incorporated by reference.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

The amino acid substitutions for each clone described in the Tables (e.g., Tables 3 and 4) dictate the amino acid substitutions at the register positions of that clone over the amino acids found in the sequence set forth in the Sequence Listing, in case of discrepancy.

All publications, patent applications, patents, and other references mentioned herein are expressly incorporated by reference in their entirety, to the same extent as if each were incorporated by reference individually. In case of conflict, the present specification, including definitions, will control.

TABLE 3
CH3C Register Positions and Mutations

| Clone | | | | | | | | | | | | | | | | | | | |
|---|---|---|---|---|---|---|---|---|---|---|---|---|---|---|---|---|---|---|---|
| name | Group | 384 | 385 | 386 | 387 | 388 | 389 | 390 | 391 | ... | 413 | 414 | 415 | 416 | 417 | 418 | 419 | 420 | 421 |
| Wild- | n/a | N | G | Q | P | E | N | N | Y | ... | D | K | S | R | W | Q | Q | G | N |
| type | | | | | | | | | | | | | | | | | | | |
| 1 | | L | G | L | V | W | V | G | Y | ... | A | K | S | T | W | Q | Q | G | W |
| 2 | | Y | G | T | V | W | S | H | Y | ... | S | K | S | E | W | Q | Q | G | Y |
| 3 | | Y | G | T | E | W | S | Q | Y | ... | E | K | S | D | W | Q | Q | G | H |
| 4 | | V | G | T | P | W | A | L | Y | ... | L | K | S | E | W | Q | Q | G | W |
| 17 | 2 | Y | G | T | V | W | S | K | Y | ... | S | K | S | E | W | Q | Q | G | F |
| 18 | 1 | L | G | H | V | W | A | V | Y | ... | P | K | S | T | W | Q | Q | G | W |
| 21 | 1 | L | G | L | V | W | V | G | Y | ... | P | K | S | T | W | Q | Q | G | W |
| 25 | 1 | M | G | H | V | W | V | G | Y | ... | D | K | S | T | W | Q | Q | G | W |
| 34 | 1 | L | G | L | V | W | V | F | S | ... | P | K | S | T | W | Q | Q | G | W |
| 35 | 2 | Y | G | T | E | W | S | S | Y | ... | T | K | S | E | W | Q | Q | G | F |
| 44 | 2 | Y | G | T | E | W | S | N | Y | ... | S | K | S | E | W | Q | Q | G | F |
| 51 | 1/2 | L | G | H | V | W | V | G | Y | ... | S | K | S | E | W | Q | Q | G | W |
| 3.1-3 | 1 | L | G | H | V | W | V | A | T | ... | P | K | S | T | W | Q | Q | G | W |
| 3.1-9 | 1 | L | G | P | V | W | V | H | T | ... | P | K | S | T | W | Q | Q | G | W |
| 3.2-5 | 1 | L | G | H | V | W | V | D | Q | ... | P | K | S | T | W | Q | Q | G | W |
| 3.2-19 | 1 | L | G | H | V | W | V | N | Q | ... | P | K | S | T | W | Q | Q | G | W |
| 3.2-1 | 1 | L | G | H | V | W | V | N | F | ... | P | K | S | T | W | Q | Q | G | W |
| 3.4-1 | | W | G | F | V | W | S | T | Y | ... | P | K | S | N | W | Q | Q | G | F |
| 3.4-19 | | W | G | H | V | W | S | T | Y | ... | P | K | S | N | W | Q | Q | G | Y |
| 3.2-3 | | L | G | H | V | W | V | E | Q | ... | P | K | S | T | W | Q | Q | G | W |
| 3.2-14 | | L | G | H | V | W | V | G | V | ... | P | K | S | T | W | Q | Q | G | W |
| 3.2-24 | | L | G | H | V | W | V | H | T | ... | P | K | S | T | W | Q | Q | G | W |
| 3.4-26 | | W | G | T | V | W | G | T | Y | ... | P | K | S | N | W | Q | Q | G | Y |
| 3.2-17 | | L | G | H | V | W | V | G | T | ... | P | K | S | T | W | Q | Q | G | W |

TABLE 4
Additional CH3C Register Positions and Mutations

| Clone name | 378 | 379 | 380 | 381 | 382 | 383 | 384 | 385 | 386 | 387 | 388 | 389 | 390 | 391 | 392 | 411 | 412 | 413 | 414 | 415 | 416 | 417 | 418 | 419 | 420 | 421 | 422 | 423 |
|---|---|---|---|---|---|---|---|---|---|---|---|---|---|---|---|---|---|---|---|---|---|---|---|---|---|---|---|---|
| Wild-type | A | V | E | W | E | S | N | G | Q | P | E | N | N | Y | K | T | V | D | K | S | R | W | Q | Q | G | N | V | F |
| 35.20.1 | . | . | . | . | . | . | F | . | T | E | W | S | S | . | . | . | . | T | . | E | E | . | . | . | . | F | . | . |
| 35.20.2 | . | . | . | . | . | . | Y | . | T | E | W | A | S | . | . | . | . | T | . | E | E | . | . | . | . | F | . | . |
| 35.20.3 | . | . | . | . | . | . | Y | . | T | E | W | V | S | . | . | . | . | T | . | E | E | . | . | . | . | F | . | . |
| 35.20.4 | . | . | . | . | . | . | Y | . | T | E | W | S | S | . | . | . | . | S | . | E | E | . | . | . | . | F | . | . |
| 35.20.5 | . | . | . | . | . | . | F | . | T | E | W | A | S | . | . | . | . | T | . | E | E | . | . | . | . | F | . | . |
| 35.20.6 | . | . | . | . | . | . | F | . | T | E | W | V | S | . | . | . | . | T | . | E | E | . | . | . | . | F | . | . |
| 35.21.a.1 | . | . | W | . | . | . | F | . | T | E | W | S | S | . | . | . | . | T | . | E | E | . | . | . | . | F | . | . |
| 35.21.a.2 | . | . | W | . | . | . | Y | . | T | E | W | A | S | . | . | . | . | T | . | E | E | . | . | . | . | F | . | . |
| 35.21.a.3 | . | . | W | . | . | . | Y | . | T | E | W | V | S | . | . | . | . | T | . | E | E | . | . | . | . | F | . | . |
| 35.21.a.4 | . | . | W | . | . | . | Y | . | T | E | W | S | S | . | . | . | . | S | . | E | E | . | . | . | . | F | . | . |
| 35.21.a.5 | . | . | W | . | . | . | F | . | T | E | W | A | S | . | . | . | . | T | . | E | E | . | . | . | . | F | . | . |
| 35.21.a.6 | . | . | W | . | . | . | F | . | T | E | W | V | S | . | . | . | . | T | . | E | E | . | . | . | . | F | . | . |
| 35.23.1 | . | . | . | . | . | . | F | . | T | E | W | S | . | . | . | . | . | T | . | E | E | . | . | . | . | F | . | . |
| 35.23.2 | . | . | . | . | . | . | Y | . | T | E | W | A | . | . | . | . | . | T | . | E | E | . | . | . | . | F | . | . |
| 35.23.3 | . | . | . | . | . | . | Y | . | T | E | W | V | . | . | . | . | . | T | . | E | E | . | . | . | . | F | . | . |
| 35.23.4 | . | . | . | . | . | . | Y | . | T | E | W | S | . | . | . | . | . | S | . | E | E | . | . | . | . | F | . | . |
| 35.23.5 | . | . | . | . | . | . | F | . | T | E | W | A | . | . | . | . | . | T | . | E | E | . | . | . | . | F | . | . |
| 35.23.6 | . | . | . | . | . | . | F | . | T | E | W | V | . | . | . | . | . | T | . | E | E | . | . | . | . | F | . | . |
| 35.24.1 | . | . | W | . | . | . | F | . | T | E | W | S | . | . | . | . | . | T | . | E | E | . | . | . | . | F | . | . |
| 35.24.2 | . | . | W | . | . | . | Y | . | T | E | W | A | . | . | . | . | . | T | . | E | E | . | . | . | . | F | . | . |
| 35.24.3 | . | . | W | . | . | . | Y | . | T | E | W | V | . | . | . | . | . | T | . | E | E | . | . | . | . | F | . | . |
| 35.24.4 | . | . | W | . | . | . | Y | . | T | E | W | S | . | . | . | . | . | S | . | E | E | . | . | . | . | F | . | . |
| 35.24.5 | . | . | W | . | . | . | F | . | T | E | W | A | . | . | . | . | . | T | . | E | E | . | . | . | . | F | . | . |
| 35.24.6 | . | . | W | . | . | . | F | . | T | E | W | V | . | . | . | . | . | T | . | E | E | . | . | . | . | F | . | . |
| 35.21.17.1 | . | . | L | . | . | . | F | . | T | E | W | S | S | . | . | . | . | T | . | E | E | . | . | . | . | F | . | . |
| 35.21.17.2 | . | . | L | . | . | . | Y | . | T | E | W | A | S | . | . | . | . | T | . | E | E | . | . | . | . | F | . | . |
| 35.21.17.3 | . | . | L | . | . | . | Y | . | T | E | W | V | S | . | . | . | . | T | . | E | E | . | . | . | . | F | . | . |
| 35.21.17.4 | . | . | L | . | . | . | Y | . | T | E | W | S | S | . | . | . | . | S | . | E | E | . | . | . | . | F | . | . |
| 35.21.17.5 | . | . | L | . | . | . | F | . | T | E | W | A | S | . | . | . | . | T | . | E | E | . | . | . | . | F | . | . |
| 35.21.17.6 | . | . | L | . | . | . | F | . | T | E | W | V | S | . | . | . | . | T | . | E | E | . | . | . | . | F | . | . |
| 35.20 | . | . | . | . | . | . | Y | . | T | E | W | S | S | . | . | . | . | T | . | E | E | . | . | . | . | F | . | . |
| 35.21 | . | . | W | . | . | . | Y | . | T | E | W | S | S | . | . | . | . | T | . | E | E | . | . | . | . | F | . | . |
| 35.22 | . | . | W | . | . | . | Y | . | T | E | W | S | . | . | . | . | . | T | . | . | E | . | . | . | . | F | . | . |
| 35.23 | . | . | . | . | . | . | Y | . | T | E | W | S | . | . | . | . | . | T | . | E | E | . | . | . | . | F | . | . |
| 35.24 | . | . | W | . | . | . | Y | . | T | E | W | S | . | . | . | . | . | T | . | E | E | . | . | . | . | F | . | . |
| 35.21.17 | . | . | L | . | . | . | Y | . | T | E | W | S | S | . | . | . | . | T | . | E | E | . | . | . | . | F | . | . |
| 35.N390 | . | . | . | . | . | . | Y | . | T | E | W | S | . | . | . | . | . | T | . | . | E | . | . | . | . | F | . | . |
| 35.20.1.1 | | | | | | | F | | T | E | W | S | S | | | | | S | | E | E | | | | | F | | |
| 35.23.2.1 | | | | | | | Y | | T | E | W | A | | | | | | S | | | E | | | | | F | | |
| 35.23.1.1 | | | | | | | F | | T | E | W | S | | | | | | S | | E | E | | | | | F | | |
| 35.S413 | | | | | | | Y | | T | E | W | S | S | | | | | S | | | E | | | | | F | | |
| 35.23.3.1 | | | | | | | Y | | T | E | W | V | | | | | | S | | E | E | | | | | F | | |
| 35.N390.1 | | | | | | | Y | | T | E | W | S | | | | | | S | | | E | | | | | F | | |
| 35.23.6.1 | | | | | | | F | | T | E | W | V | | | | | | S | | E | E | | | | | F | | |

### INFORMAL SEQUENCE LISTING

| SEQ ID | | |
|---|---|---|
| NO: | Sequence | Description |
| 1 | APELLGGPSVFLFPPKPKDTLMISRTPEVTCVVVDVSHEDPEVKFNW | Wild-type human Fc |
| | YVDGVEVHNAKTKPREEQYNSTYRVVSVLTVLHQDWLNGKEYKCK | sequence |
| | VSNKALPAPIEKTISKAKGQPREPQVYTLPPSRDELTKNQVSLTCLVK | |
| | GFYPSDIAVEWESNGQPENNYKTTPPVLDSDGSFFLYSKLTVDKSRW | |
| | QQGNVFSCSVMHEALHNHYTQKSLSLSPGK | |
| 2 | APELLGGPSVFLFPPKPKDTLMISRTPEVTCVVVDVSHEDPEVKFNW | CH2 domain sequence |
| | YVDGVEVHNAKTKPREEQYNSTYRVVSVLTVLHQDWLNGKEYKCK | |
| | VSNKALPAPIEKTISKAK | |
| 3 | GQPREPQVYTLPPSRDELTKNQVSLTCLVKGFYPSDIAVEWESNGQP | CH3 domain sequence |
| | ENNYKTTPPVLDSDGSFFLYSKLTVDKSRWQQGNVFSCSVMHEALH | |
| | NHYTQKSLSLSPGK | |
| 4 | APELLGGPSVFLFPPKPKDTLMISRTPEVTCVVVDVSHEDPEVKFNW | Clone CH3C.1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGLVWVGYKTTPPVLDSDGSFFLYSKLTVAKSTW | |
| | QQGWVFSCSVMHEALHNHYTQKSLSLSPGK | |
| 5 | APELLGGPSVFLFPPKPKDTLMISRTPEVTCVVVDVSHEDPEVKFNW | Clone CH3C.2 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTVWSHYKTTPPVLDSDGSFFLYSKLTVSKSEW | |
| | QQGYVFSCSVMHEALHNHYTQKSLSLSPGK | |
| 6 | APELLGGPSVFLFPPKPKDTLMISRTPEVTCVVVDVSHEDPEVKFNW | Clone CH3C.3 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWSQYKTTPPVLDSDGSFFLYSKLTVEKSDW | |
| | QQGHVFSCSVMHEALHNHYTQKSLSLSPGK | |
| 7 | APELLGGPSVFLFPPKPKDTLMISRTPEVTCVVVDVSHEDPEVKFNW | Clone CH3C.4 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESVGTPWALYKTTPPVLDSDGSFFLYSKLTVLKSEW | |
| | QQGWVFSCSVMHEALHNHYTQKSLSLSPGK | |
| 8 | APELLGGPSVFLFPPKPKDTLMISRTPEVTCVVVDVSHEDPEVKFNW | Clone CH3C.17 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTVWSKYKTTPPVLDSDGSFFLYSKLTVSKSEW | |
| | QQGFVFSCSVMHEALHNHYTQKSLSLSPGK | |
| 9 | APELLGGPSVFLFPPKPKDTLMISRTPEVTCVVVDVSHEDPEVKFNW | Clone CH3C.18 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGHVWAVYKTTPPVLDSDGSFFLYSKLTVPKSTW | |
| | QQGWVFSCSVMHEALHNHYTQKSLSLSPGK | |
| 10 | APELLGGPSVFLFPPKPKDTLMISRTPEVTCVVVDVSHEDPEVKFNW | Clone CH3C.21 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGLVWVGYKTTPPVLDSDGSFFLYSKLTVPKSTW | |
| | QQGWVFSCSVMHEALHNHYTQKSLSLSPGK | |
| 11 | APELLGGPSVFLFPPKPKDTLMISRTPEVTCVVVDVSHEDPEVKFNW | Clone CH3C.25 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESMGHVWVGYKTTPPVLDSDGSFFLYSKLTVDKST | |
| | WQQGWVFSCSVMHEALHNHYTQKSLSLSPGK | |
| 12 | APELLGGPSVFLFPPKPKDTLMISRTPEVTCVVVDVSHEDPEVKFNW | Clone CH3C.34 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGLVWVFSKTTPPVLDSDGSFFLYSKLTVPKSTW | |
| | QQGWVFSCSVMHEALHNHYTQKSLSLSPGK | |
| 13 | APELLGGPSVFLFPPKPKDTLMISRTPEVTCVVVDVSHEDPEVKFNW | Clone CH3C.35 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWSSYKTTPPVLDSDGSFFLYSKLTVTKSEW | |
| | QQGFVFSCSVMHEALHNHYTQKSLSLSPGK | |
| 14 | APELLGGPSVFLFPPKPKDTLMISRTPEVTCVVVDVSHEDPEVKFNW | Clone CH3C.44 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWSNYKTTPPVLDSDGSFFLYSKLTVSKSEW | |
| | QQGFVFSCSVMHEALHNHYTQKSLSLSPGK | |
| 15 | APELLGGPSVFLFPPKPKDTLMISRTPEVTCVVVDVSHEDPEVKFNW | Clone CH3C.51 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGHVWVGYKTTPPVLDSDGSFFLYSKLTVSKSEW | |
| | QQGWVFSCSVMHEALHNHYTQKSLSLSPGK | |
| 16 | APELLGGPSVFLFPPKPKDTLMISRTPEVTCVVVDVSHEDPEVKFNW | Clone CH3C.3.1-3 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGHVWVATKTTPPVLDSDGSFFLYSKLTVPKSTW | |
| | QQGWVFSCSVMHEALHNHYTQKSLSLSPGK | |
| 17 | APELLGGPSVFLFPPKPKDTLMISRTPEVTCVVVDVSHEDPEVKFNW | Clone CH3C.3.1-9 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGPVWVHTKTTPPVLDSDGSFFLYSKLTVPKSTW | |
| | QQGWVFSCSVMHEALHNHYTQKSLSLSPGK | |
| 18 | APELLGGPSVFLFPPKPKDTLMISRTPEVTCVVVDVSHEDPEVKFNW | Clone CH3C.3.2-5 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGHVWVDQKTTPPVLDSDGSFFLYSKLTVPKSTW | |
| | QQGWVFSCSVMHEALHNHYTQKSLSLSPGK | |
| 19 | APELLGGPSVFLFPPKPKDTLMISRTPEVTCVVVDVSHEDPEVKFNW | Clone CH3C.3.2-19 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGHVWVNQKTTPPVLDSDGSFFLYSKLTVPKSTW | |
| | QQGWVFSCSVMHEALHNHYTQKSLSLSPGK | |
| 20 | APELLGGPSVFLFPPKPKDTLMISRTPEVTCVVVDVSHEDPEVKFNW | Clone CH3C.3.2-1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGHVWVNFKTTPPVLDSDGSFFLYSKLTVPKSTW | |
| | QQGWVFSCSVMHEALHNHYTQKSLSLSPGK | |
| 21 | APELLGGPSVFLFPPKPKDTLMISRTPEVTCVVVDVSHEDPEVKFNW | Clone CH3C.18.E153W |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESLGHVWAVYKTTPPVLDSDGSFFLYSKLTVPKST | |
| | WQQGWVFSCSVMHEALHNHYTQKSLSLSPGK | |
| 22 | APELLGGPSVFLFPPKPKDTLMISRTPEVTCVVVDVSHEDPEVKFNW | Clone CH3C.18.K165Q |
| | YVDGVEVHNAKTKPREEQYNSTYRVVSVLTVLHQDWLNGKEYKCK | (CH3C.35.14) |
| | VSNKALPAPIEKTISKAKGQPREPQVYTLPPSRDELTKNQVSLTCLVK | |
| | GFYPSDIAVEWESLGHVWAVYQTTPPVLDSDGSFFLYSKLTVPKSTW | |
| | QQGWVFSCSVMHEALHNHYTQKSLSLSPGK | |
| 23 | APELLGGPSVFLFPPKPKDTLMISRTPEVTCVVVDVSHEDPEVKFNW | Clone CH3C.18.E153W. |
| | YVDGVEVHNAKTKPREEQYNSTYRVVSVLTVLHQDWLNGKEYKCK | K165Q (CH3C.35.15) |
| | VSNKALPAPIEKTISKAKGQPREPQVYTLPPSRDELTKNQVSLTCLVK | |
| | GFYPSDIAVWWESLGHVWAVYQTTPPVLDSDGSFFLYSKLTVPKST | |
| | WQQGWVFSCSVMHEALHNHYTQKSLSLSPGK | |
| 24 | APELLGGPSVFLFPPKPKDTLMISRTPEVTCVVVDVSHEDPEVKFNW | Clone CH3C.35.E153W |
| | YVDGVEVHNAKTKPREEQYNSTYRVVSVLTVLHQDWLNGKEYKCK | (CH3C.35.19) |
| | VSNKALPAPIEKTISKAKGQPREPQVYTLPPSRDELTKNQVSLTCLVK | |
| | GFYPSDIAVWWESYGTEWSSYKTTPPVLDSDGSFFLYSKLTVTKSEW | |
| | QQGFVFSCSVMHEALHNHYTQKSLSLSPGK | |
| 25 | APELLGGPSVFLFPPKPKDTLMISRTPEVTCVVVDVSHEDPEVKFNW | Clone CH3C.35.S188E |
| | YVDGVEVHNAKTKPREEQYNSTYRVVSVLTVLHQDWLNGKEYKCK | (CH3C.35.20) |
| | VSNKALPAPIEKTISKAKGQPREPQVYTLPPSRDELTKNQVSLTCLVK | |
| | GFYPSDIAVEWESYGTEWSSYKTTPPVLDSDGSFFLYSKLTVTKEEW | |
| | QQGFVFSCSVMHEALHNHYTQKSLSLSPGK | |
| 26 | APELLGGPSVFLFPPKPKDTLMISRTPEVTCVVVDVSHEDPEVKFNW | Clone CH3C.35.E153W. |
| | YVDGVEVHNAKTKPREEQYNSTYRVVSVLTVLHQDWLNGKEYKCK | S188E (CH3C.35.21) |
| | VSNKALPAPIEKTISKAKGQPREPQVYTLPPSRDELTKNQVSLTCLVK | |
| | GFYPSDIAVWWESYGTEWSSYKTTPPVLDSDGSFFLYSKLTVTKEEW | |
| | QQGFVFSCSVMHEALHNHYTQKSLSLSPGK | |
| 27 | APELLGGPSVFLFPPKPKDTLMISRTPEVTCVVVDVSHEDPEVKFNW | Clone CH3C.35.N163 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWSNYKTTPPVLDSDGSFFLYSKLTVTKSEW | |
| | QQGFVFSCSVMHEALHNHYTQKSLSLSPGK | |
| 28 | APELLGGPSVFLFPPKPKDTLMISRTPEVTCVVVDVSHEDPEVKFNW | Clone CH3C.35.K165Q |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWSSYQTTPPVLDSDGSFFLYSKLTVTKSEW | |
| | QQGFVFSCSVMHEALHNHYTQKSLSLSPGK | |
| 29 | APELLGGPSVFLFPPKPKDTLMISRTPEVTCVVVDVSHEDPEVKFNW | Clone CH3C.35.N163. |
| | YVDGVEVHNAKTKPREEQYNSTYRVVSVLTVLHQDWLNGKEYKCK | K165Q |
| | VSNKALPAPIEKTISKAKGQPREPQVYTLPPSRDELTKNQVSLTCLVK | |
| | GFYPSDIAVEWESYGTEWSNYQTTPPVLDSDGSFFLYSKLTVTKSEW | |
| | QQGFVFSCSVMHEALHNHYTQKSLSLSPGK | |
| 30 | APELLGGPSVFLFPPKPKDTLMISRTPEVTCVVVDVSHEDPEVKFNW | CH3C library (X denotes |
| | YVDGVEVHNAKTKPREEQYNSTYRVVSVLTVLHQDWLNGKEYKCK | randomized amino acid |
| | VSNKALPAPIEKTISKAKGQPREPQVYTLPPSRDELTKNQVSLTCLVK | position) |
| | GFYPSDIAVEWESXGXXXXXYKTTPPVLDSDGSFFLYSKLTVXKXX | |
| | WQQGXVFSCSVMHEALHNHYTQKSLSLSPGK | |
| 31 | NSVIIVDKNGRLVYLVENPGGYVAYSKAATVTGKLVHANFGTKKDF | Human TfR apical domain |
| | EDLYTPVNGSIVIVRAGKITFAEKVANAESLNAIGVLIYMDQTKFPIV | |
| | NAELSFFGHAHLGTGDPYTPGFPSFNHTQFPPSRSSGLPNIPVQTISRA | |
| | AAEKLFGNMEGDCPSDWKTDSTCRMVTSESKNVKLTVS | |
| 32 | NSVIIVDKNGGLVYLVENPGGYVAYSKAATVTGKLVHANFGTKKDF | Cynomolgus TfR apical |
| | EDLDSPVNGSIVIVRAGKITFAEKVANAESLNAIGVLIYMDQTKFPIV | domain |
| | KADLSFFGHAHLGTGDPYTPGFPSFNHTQFPPSQSSGLPNIPVQTISRA | |
| | AAEKLFGNMEGDCPSDWKTDSTCKMVTSENKSVKLTVS | |
| 33 | SSGLPNIPVQTISRAAAEKLFGNMEGDCPSDWKTDSTCRMVTSESKN | Loop-truncated human |
| | VKLTVSNDSAQNSVIIVDKNGRLVYLVENPGGYVAYSKAATVTGKL | TfR apical domain |
| | VHANFGTKKDFEDLYTPVNGSIVIVRAGKITFAEKVANAESLNAIGVL | displayed on phage |
| | IYMDQTKFPIVNAELSGP | |
| 34 | SSGLPNIPVQTISRAAAEKLFGNMEGDCPSDWKTDSTCKMVTSENKS | Loop-truncated |
| | VKLTVSNDSAQNSVIIVDKNGGLVYLVENPGGYVAYSKAATVTGKL | cynomolgus TfR apical |
| | VHANFGTKKDFEDLDSPVNGSIVIVRAGKITFAEKVANAESLNAIGVL | domain displayed on |
| | IYMDQTKFPIVKADLSGP | phage |
| 35 | WESXGXXXXXYK | First portion CH3C |
| | | register |
| 36 | TVMOOCWQQGXV | Second portion CH3C |
| | | register |
| 37 | YGTEW | CH3C conserved sequence |
| 38 | LGLVWVG | CH3C modified binding |
| | | sequence |
| 39 | YGTVWSH | CH3C modified binding |
| | | sequence |
| 40 | YGTEWSQ | CH3C modified binding |
| | | sequence |
| 41 | VGTPWAL | CH3C modified binding |
| | | sequence |
| 42 | YGTVWSK | CH3C modified binding |
| | | sequence |
| 43 | LGHVWAV | CH3C modified binding |
| | | sequence |
| 44 | MGHVWVG | CH3C modified binding |
| | | sequence |
| 45 | LGLVGVF | CH3C modified binding |
| | | sequence |
| 46 | YGTEWSS | CH3C modified binding |
| | | sequence |
| 47 | YGTEWSN | CH3C modified binding |
| | | sequence |
| 48 | LGHVWVG | CH3C modified binding |
| | | sequence |
| 49 | LGHVWVA | CH3C modified binding |
| | | sequence |
| 50 | LGPVWVH | CH3C modified binding |
| | | sequence |
| 51 | LGHVWVD | CH3C modified binding |
| | | sequence |
| 52 | LGHVWVN | CH3C modified binding |
| | | sequence |
| 53 | AKSTWQQGW | CH3C modified binding |
| | | sequence |
| 54 | SKSEWQQGY | CH3C modified binding |
| | | sequence |
| 55 | EKSDWQQGH | CH3C modified binding |
| | | sequence |
| 56 | LKSEWQQGW | CH3C modified binding |
| | | sequence |
| 57 | SKSEWQQGF | CH3C modified binding |
| | | sequence |
| 58 | PKSTWQQGW | CH3C modified binding |
| | | sequence |
| 59 | DKSTWQQGW | CH3C modified binding |
| | | sequence |
| 60 | TKSEWQQGF | CH3C modified binding |
| | | sequence |
| 61 | SKSEWQQGW | CH3C modified binding |
| | | sequence |
| 62 | EPKSCDKTHTCPPCP | Human IgG1 hinge amino |
| | | acid sequence |
| 63 | MMDQARSAFSNLFGGEPLSYTRFSLARQVDGDNSHVEMKLAVDEEE | Human transferrin receptor |
| | NADNNTKANVTKPKRCSGSICYGTIAVIVFFLIGFMIGYLGYCKGVEP | protein 1 (TFR1) |
| | KTECERLAGTESPVREEPGEDFPAARRLYWDDLKRKLSEKLDSTDFT | |
| | GTIKLLNENSYVPREAGSQKDENLALYVENQFREFKLSKVWRDQHF | |
| | VKIQVKDSAQNSVIIVDKNGRLVYLVENPGGYVAYSKAATVTGKLV | |
| | HANFGTKKDFEDLYTPVNGSIVIVRAGKITFAEKVANAESLNAIGVLI | |
| | YMDQTKFPIVNAELSFFGHAHLGTGDPYTPGFPSFNHTQFPPSRSSGL | |
| | PNIPVQTISRAAAEKLFGNMEGDCPSDWKTDSTCRMVTSESKNVKLT | |
| | VSNVLKEIKILNIFGVIKGFVEPDHYVVVGAQRDAWGPGAAKSGVGT | |
| | ALLLKLAQMFSDMVLKDGFQPSRSIIFASWSAGDFGSVGAIEWLEGY | |
| | LSSLHLKAFTYINLDKAVLGTSNFKVSASPLLYTLIEKTMQNVKHPVT | |
| | GQFLYQDSNWASKVEKLTLDNAAFPFLAYSGIPAVSFCFCEDTDYPY | |
| | LGTTMDTYKELIERIPELNKVARAAAEVAGQFVIKLTHDVELNLDYE | |
| | RYNSQLLSFVRDLNQYRADIKEMGLSLQWLYSARGDFFRATSRLTTD | |
| | FGNAEKTDRFVMKKLNDRVMRVEYHFLSPYVSPKESPFRHVFWGSG | |
| | SHTLPALLENLKLRKQNNGAFNETLFRNQLALATWTIQGAANALSG | |
| | DVWDIDNEF | |
| 64 | APELLGGPSVFLFPPKPKDTLMISRTPEVTCVVVDVSHEDPEVKFNW | Clone CH3C.35.19 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWSSYKTTPPVLDSDGSFFLYSKLTVTKSEW | |
| | QQGFVFSCSVMHEALHNHYTQKSLSLSPGK | |
| 65 | APELLGGPSVFLFPPKPKDTLMISRTPEVTCVVVDVSHEDPEVKFNW | Clone CH3C.35.20 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWSSYKTTPPVLDSDGSFFLYSKLTVTKEEW | |
| | QQGFVFSCSVMHEALHNHYTQKSLSLSPGK | |
| 66 | APELLGGPSVFLFPPKPKDTLMISRTPEVTCVVVDVSHEDPEVKFNW | Clone CH3C.35.21 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWSSYKTTPPVLDSDGSFFLYSKLTVTKEEW | |
| | QQGFVFSCSVMHEALHNHYTQKSLSLSPGK | |
| 67 | APELLGGPSVFLFPPKPKDTLMISRTPEVTCVVVDVSHEDPEVKFNW | Clone CH3C.35.22 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWSNYKTTPPVLDSDGSFFLYSKLTVTKSEW | |
| | QQGFVFSCSVMHEALHNHYTQKSLSLSPGK | |
| 68 | APELLGGPSVFLFPPKPKDTLMISRTPEVTCVVVDVSHEDPEVKFNW | Clone CH3C.35.23 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWSNYKTTPPVLDSDGSFFLYSKLTVTKEEW | |
| | QQGFVFSCSVMHEALHNHYTQKSLSLSPGK | |
| 69 | APELLGGPSVFLFPPKPKDTLMISRTPEVTCVVVDVSHEDPEVKFNW | Clone CH3C.35.24 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWSNYKTTPPVLDSDGSFFLYSKLTVTKEE | |
| | WQQGFVFSCSVMHEALHNHYTQKSLSLSPGK | |
| 70 | APELLGGPSVFLFPPKPKDTLMISRTPEVTCVVVDVSHEDPEVKFNW | CH3C.18 variant |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESLGHVWAVYKTTPPVLDSDGSFFLYSKLTVPKST | |
| | WQQGWVFSCSVMHEALHNHYTQKSLSLSPGK | |
| 71 | APELLGGPSVFLFPPKPKDTLMISRTPEVTCVVVDVSHEDPEVKFNW | CH3C.18 variant |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESLGHVWAVYKTTPPVLDSDGSFFLYSKLTVPKSTW | |
| | QQGWVFSCSVMHEALHNHYTQKSLSLSPGK | |
| 72 | APELLGGPSVFLFPPKPKDTLMISRTPEVTCVVVDVSHEDPEVKFNW | CH3C.18 variant |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVYWESLGHVWAVYKTTPPVLDSDGSFFLYSKLTVPKSTW | |
| | QQGWVFSCSVMHEALHNHYTQKSLSLSPGK | |
| 73 | APELLGGPSVFLFPPKPKDTLMISRTPEVTCVVVDVSHEDPEVKFNW | CH3C.18 variant |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGHVWAVYQTTPPVLDSDGSFFLYSKLTVPKSTW | |
| | QQGWVFSCSVMHEALHNHYTQKSLSLSPGK | |
| 74 | APELLGGPSVFLFPPKPKDTLMISRTPEVTCVVVDVSHEDPEVKFNW | CH3C.18 variant |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGHVWAVYFTTPPVLDSDGSFFLYSKLTVPKSTW | |
| | QQGWVFSCSVMHEALHNHYTQKSLSLSPGK | |
| 75 | APELLGGPSVFLFPPKPKDTLMISRTPEVTCVVVDVSHEDPEVKFNW | CH3C.18 variant |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGHVWAVYHTTPPVLDSDGSFFLYSKLTVPKSTW | |
| | QQGWVFSCSVMHEALHNHYTQKSLSLSPGK | |
| 76 | APELLGGPSVFLFPPKPKDTLMISRTPEVTCVVVDVSHEDPEVKFNW | Clone CH3C.35.21.1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYKTTPPVLDSDGSFFLYSKLTVTKSEW | |
| | QQGFVFSCSVMHEALHNHYTQKSLSLSPGK | |
| 77 | APELLGGPSVFLFPPKPKDTLMISRTPEVTCVVVDVSHEDPEVKFNW | Clone CH3C.35.21.2 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYRTTPPVLDSDGSFFLYSKLTVTKSEW | |
| | QQGFVFSCSVMHEALHNHYTQKSLSLSPGK | |
| 78 | APELLGGPSVFLFPPKPKDTLMISRTPEVTCVVVDVSHEDPEVKFNW | Clone CH3C.35.21.3 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYRTTPPVLDSDGSFFLYSKLTVTREEW | |
| | QQGFVFSCSVMHEALHNHYTQKSLSLSPGK | |
| 79 | APELLGGPSVFLFPPKPKDTLMISRTPEVTCVVVDVSHEDPEVKFNW | Clone CH3C.35.21.4 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYRTTPPVLDSDGSFFLYSKLTVTGEEW | |
| | QQGFVFSCSVMHEALHNHYTQKSLSLSPGK | |
| 80 | APELLGGPSVFLFPPKPKDTLMISRTPEVTCVVVDVSHEDPEVKFNW | Clone CH3C.35.21.5 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYRTTPPVLDSDGSFFLYSKLTVTREEW | |
| | QQGFVFSCWVMHEALHNHYTQKSLSLSPGK | |
| 81 | APELLGGPSVFLFPPKPKDTLMISRTPEVTCVVVDVSHEDPEVKFNW | Clone CH3C.35.21.6 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYRTTPPVLDSDGSFFLYSKLTVTKEEW | |
| | QQGFVFSCWVMHEALHNHYTQKSLSLSPGK | |
| 82 | APELLGGPSVFLFPPKPKDTLMISRTPEVTCVVVDVSHEDPEVKFNW | Clone CH3C.35.21.7 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYRTTPPVLDSDGSFFLYSKLTVTREEW | |
| | QQGFVFTCWVMHEALHNHYTQKSLSLSPGK | |
| 83 | APELLGGPSVFLFPPKPKDTLMISRTPEVTCVVVDVSHEDPEVKFNW | Clone CH3C.35.21.8 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYRTTPPVLDSDGSFFLYSKLTVTREEW | |
| | QQGFVFTCGVMHEALHNHYTQKSLSLSPGK | |
| 84 | APELLGGPSVFLFPPKPKDTLMISRTPEVTCVVVDVSHEDPEVKFNW | Clone CH3C.35.21.9 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYRTTPPVLDSDGSFFLYSKLTVTREEW | |
| | QQGFVFECWVMHEALHNHYTQKSLSLSPGK | |
| 85 | APELLGGPSVFLFPPKPKDTLMISRTPEVTCVVVDVSHEDPEVKFNW | Clone CH3C.35.21.10 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYRTTPPVLDSDGSFFLYSKLTVTREEW | |
| | QQGFVFKCWVMHEALHNHYTQKSLSLSPGK | |
| 86 | APELLGGPSVFLFPPKPKDTLMISRTPEVTCVVVDVSHEDPEVKFNW | Clone CH3C.35.21.11 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYRTTPPVLDSDGSFFLYSKLTVTPEEW | |
| | QQGFVFKCWVMHEALHNHYTQKSLSLSPGK | |
| 87 | APELLGGPSVFLFPPKPKDTLMISRTPEVTCVVVDVSHEDPEVKFNW | Clone CH3C.35.21.12 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWSSYRTTPPVLDSDGSFFLYSKLTVTREEW | |
| | QQGFVFSCSVMHEALHNHYTQKSLSLSPGK | |
| 88 | APELLGGPSVFLFPPKPKDTLMISRTPEVTCVVVDVSHEDPEVKFNW | Clone CH3C.35.21.13 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWSSYRTTPPVLDSDGSFFLYSKLTVTGEEW | |
| | QQGFVFSCSVMHEALHNHYTQKSLSLSPGK | |
| 89 | APELLGGPSVFLFPPKPKDTLMISRTPEVTCVVVDVSHEDPEVKFNW | Clone CH3C.35.21.14 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWSSYRTTPPVLDSDGSFFLYSKLTVTREEW | |
| | QQGFVFTCWVMHEALHNHYTQKSLSLSPGK | |
| 90 | APELLGGPSVFLFPPKPKDTLMISRTPEVTCVVVDVSHEDPEVKFNW | Clone CH3C.35.21.15 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWSSYRTTPPVLDSDGSFFLYSKLTVTGEEW | |
| | QQGFVFTCWVMHEALHNHYTQKSLSLSPGK | |
| 91 | APELLGGPSVFLFPPKPKDTLMISRTPEVTCVVVDVSHEDPEVKFNW | Clone CH3C.35.21.16 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWSSYRTTPPVLDSDGSFFLYSKLTVTREEW | |
| | QQGFVFTCGVMHEALHNHYTQKSLSLSPGK | |
| 92 | APELLGGPSVFLFPPKPKDTLMISRTPEVTCVVVDVSHEDPEVKFNW | Clone CH3C.35.21.17 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYKTTPPVLDSDGSFFLYSKLTVTKEEW | |
| | QQGFVFSCSVMHEALHNHYTQKSLSLSPGK | |
| 93 | APELLGGPSVFLFPPKPKDTLMISRTPEVTCVVVDVSHEDPEVKFNW | Clone CH3C.35.21.18 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYRTTPPVLDSDGSFFLYSKLTVTKEEW | |
| | QQGFVFSCSVMHEALHNHYTQKSLSLSPGK | |
| 94 | APELLGGPSVFLFPPKPKDTLMISRTPEVTCVVVDVSHEDPEVKFNW | Clone CH3C.35.20.1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESFGTEWSSYKTTPPVLDSDGSFFLYSKLTVTKEEW | |
| | QQGFVFSCSVMHEALHNHYTQKSLSLSPGK | |
| 95 | APELLGGPSVFLFPPKPKDTLMISRTPEVTCVVVDVSHEDPEVKFNW | Clone CH3C.35.20.2 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWASYKTTPPVLDSDGSFFLYSKLTVTKEEW | |
| | QQGFVFSCSVMHEALHNHYTQKSLSLSPGK | |
| 96 | APELLGGPSVFLFPPKPKDTLMISRTPEVTCVVVDVSHEDPEVKFNW | Clone CH3C.35.20.3 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWVSYKTTPPVLDSDGSFFLYSKLTVTKEEW | |
| | QQGFVFSCSVMHEALHNHYTQKSLSLSPGK | |
| 97 | APELLGGPSVFLFPPKPKDTLMISRTPEVTCVVVDVSHEDPEVKFNW | Clone CH3C.35.20.4 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWSSYKTTPPVLDSDGSFFLYSKLTVSKEEW | |
| | QQGFVFSCSVMHEALHNHYTQKSLSLSPGK | |
| 98 | APELLGGPSVFLFPPKPKDTLMISRTPEVTCVVVDVSHEDPEVKFNW | Clone CH3C.35.20.5 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESFGTEWASYKTTPPVLDSDGSFFLYSKLTVTKEEW | |
| | QQGFVFSCSVMHEALHNHYTQKSLSLSPGK | |
| 99 | APELLGGPSVFLFPPKPKDTLMISRTPEVTCVVVDVSHEDPEVKFNW | Clone CH3C.35.20.6 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESFGTEWVSYKTTPPVLDSDGSFFLYSKLTVTKEEW | |
| | QQGFVFSCSVMHEALHNHYTQKSLSLSPGK | |
| 100 | APELLGGPSVFLFPPKPKDTLMISRTPEVTCVVVDVSHEDPEVKFNW | Clone CH3C.35.21.a.1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESFGTEWSSYKTTPPVLDSDGSFFLYSKLTVTKEEW | |
| | QQGFVFSCSVMHEALHNHYTQKSLSLSPGK | |
| 101 | APELLGGPSVFLFPPKPKDTLMISRTPEVTCVVVDVSHEDPEVKFNW | Clone CH3C.35.21.a.2 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWASYKTTPPVLDSDGSFFLYSKLTVTKEE | |
| | WQQGFVFSCSVMHEALHNHYTQKSLSLSPGK | |
| 102 | APELLGGPSVFLFPPKPKDTLMISRTPEVTCVVVDVSHEDPEVKFNW | Clone CH3C.35.21.a.3 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWVSYKTTPPVLDSDGSFFLYSKLTVTKEE | |
| | WQQGFVFSCSVMHEALHNHYTQKSLSLSPGK | |
| 103 | APELLGGPSVFLFPPKPKDTLMISRTPEVTCVVVDVSHEDPEVKFNW | Clone CH3C.35.21.a.4 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWSSYKTTPPVLDSDGSFFLYSKLTVSKEEW | |
| | QQGFVFSCSVMHEALHNHYTQKSLSLSPGK | |
| 104 | APELLGGPSVFLFPPKPKDTLMISRTPEVTCVVVDVSHEDPEVKFNW | Clone CH3C.35.21.a.5 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESFGTEWASYKTTPPVLDSDGSFFLYSKLTVTKEEW | |
| | QQGFVFSCSVMHEALHNHYTQKSLSLSPGK | |
| 105 | APELLGGPSVFLFPPKPKDTLMISRTPEVTCVVVDVSHEDPEVKFNW | Clone CH3C.35.21.a.6 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESFGTEWVSYKTTPPVLDSDGSFFLYSKLTVTKEEW | |
| | QQGFVFSCSVMHEALHNHYTQKSLSLSPGK | |
| 106 | APELLGGPSVFLFPPKPKDTLMISRTPEVTCVVVDVSHEDPEVKFNW | Clone CH3C.35.23.1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESFGTEWSNYKTTPPVLDSDGSFFLYSKLTVTKEEW | |
| | QQGFVFSCSVMHEALHNHYTQKSLSLSPGK | |
| 107 | APELLGGPSVFLFPPKPKDTLMISRTPEVTCVVVDVSHEDPEVKFNW | Clone CH3C.35.23.2 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWANYKTTPPVLDSDGSFFLYSKLTVTKEEW | |
| | QQGFVFSCSVMHEALHNHYTQKSLSLSPGK | |
| 108 | APELLGGPSVFLFPPKPKDTLMISRTPEVTCVVVDVSHEDPEVKFNW | Clone CH3C.35.23.3 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWVNYKTTPPVLDSDGSFFLYSKLTVTKEEW | |
| | QQGFVFSCSVMHEALHNHYTQKSLSLSPGK | |
| 109 | APELLGGPSVFLFPPKPKDTLMISRTPEVTCVVVDVSHEDPEVKFNW | Clone CH3C.35.23.4 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWSNYKTTPPVLDSDGSFFLYSKLTVSKEEW | |
| | QQGFVFSCSVMHEALHNHYTQKSLSLSPGK | |
| 110 | APELLGGPSVFLFPPKPKDTLMISRTPEVTCVVVDVSHEDPEVKFNW | Clone CH3C.35.23.5 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESFGTEWANYKTTPPVLDSDGSFFLYSKLTVTKEEW | |
| | QQGFVFSCSVMHEALHNHYTQKSLSLSPGK | |
| 111 | APELLGGPSVFLFPPKPKDTLMISRTPEVTCVVVDVSHEDPEVKFNW | Clone CH3C.35.23.6 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESFGTEWVNYKTTPPVLDSDGSFFLYSKLTVTKEEW | |
| | QQGFVFSCSVMHEALHNHYTQKSLSLSPGK | |
| 112 | APELLGGPSVFLFPPKPKDTLMISRTPEVTCVVVDVSHEDPEVKFNW | Clone CH3C.35.24.1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESFGTEWSNYKTTPPVLDSDGSFFLYSKLTVTKEEW | |
| | QQGFVFSCSVMHEALHNHYTQKSLSLSPGK | |
| 113 | APELLGGPSVFLFPPKPKDTLMISRTPEVTCVVVDVSHEDPEVKFNW | Clone CH3C.35.24.2 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWANYKTTPPVLDSDGSFFLYSKLTVTKEE | |
| | WQQGFVFSCSVMHEALHNHYTQKSLSLSPGK | |
| 114 | APELLGGPSVFLFPPKPKDTLMISRTPEVTCVVVDVSHEDPEVKFNW | Clone CH3C.35.24.3 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWVNYKTTPPVLDSDGSFFLYSKLTVTKEE | |
| | WQQGFVFSCSVMHEALHNHYTQKSLSLSPGK | |
| 115 | APELLGGPSVFLFPPKPKDTLMISRTPEVTCVVVDVSHEDPEVKFNW | Clone CH3C.35.24.4 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESYGTEWSNYKTTPPVLDSDGSFFLYSKLTVSKEEW | |
| | QQGFVFSCSVMHEALHNHYTQKSLSLSPGK | |
| 116 | APELLGGPSVFLFPPKPKDTLMISRTPEVTCVVVDVSHEDPEVKFNW | Clone CH3C.35.24.5 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESFGTEWANYKTTPPVLDSDGSFFLYSKLTVTKEE | |
| | WQQGFVFSCSVMHEALHNHYTQKSLSLSPGK | |
| 117 | APELLGGPSVFLFPPKPKDTLMISRTPEVTCVVVDVSHEDPEVKFNW | Clone CH3C.35.24.6 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESFGTEWVNYKTTPPVLDSDGSFFLYSKLTVTKEE | |
| | WQQGFVFSCSVMHEALHNHYTQKSLSLSPGK | |
| 118 | APELLGGPSVFLFPPKPKDTLMISRTPEVTCVVVDVSHEDPEVKFNW | Clone CH3C.35.21.17.1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESFGTEWSSYKTTPPVLDSDGSFFLYSKLTVTKEEW | |
| | QQGFVFSCSVMHEALHNHYTQKSLSLSPGK | |
| 119 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWASYKTTPPVLDSDGSFFLYSKLTVTKEEW | |
| | QQGFVFSCSVMHEALHNHYTQKSLSLSPGK | |
| 120 | APELLGGPSVFLFPPKPKDTLMISRTPEVTCVVVDVSHEDPEVKFNW | Clone CH3C.35.21.17.3 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWVSYKTTPPVLDSDGSFFLYSKLTVTKEEW | |
| | QQGFVFSCSVMHEALHNHYTQKSLSLSPGK | |
| 121 | APELLGGPSVFLFPPKPKDTLMISRTPEVTCVVVDVSHEDPEVKFNW | Clone CH3C.35.21.17.4 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESYGTEWSSYKTTPPVLDSDGSFFLYSKLTVSKEEW | |
| | QQGFVFSCSVMHEALHNHYTQKSLSLSPGK | |
| 122 | APELLGGPSVFLFPPKPKDTLMISRTPEVTCVVVDVSHEDPEVKFNW | Clone CH3C.35.21.17.5 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESFGTEWASYKTTPPVLDSDGSFFLYSKLTVTKEEW | |
| | QQGFVFSCSVMHEALHNHYTQKSLSLSPGK | |
| 123 | APELLGGPSVFLFPPKPKDTLMISRTPEVTCVVVDVSHEDPEVKFNW | Clone CH3C.35.21.17.6 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVLWESFGTEWVSYKTTPPVLDSDGSFFLYSKLTVTKEEW | |
| | QQGFVFSCSVMHEALHNHYTQKSLSLSPGK | |
| 124 | APELLGGPSVFLFPPKPKDTLMISRTPEVTCVVVDVSHEDPEVKFNW | Clone CH3C.35.N390 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWSNYKTTPPVLDSDGSFFLYSKLTVTKSEW | |
| | QQGFVFSCSVMHEALHNHYTQKSLSLSPGK | |
| 125 | APELLGGPSVFLFPPKPKDTLMISRTPEVTCVVVDVSHEDPEVKFNW | Clone CH3C.35.16 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESLGHVWVNQKTTPPVLDSDGSFFLYSKLTVPKST | |
| | WQQGWVFSCSVMHEALHNHYTQKSLSLSPGK | |
| 126 | APELLGGPSVFLFPPKPKDTLMISRTPEVTCVVVDVSHEDPEVKFNW | Clone CH3 C.35.17 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESLGHVWVNQQTTPPVLDSDGSFFLYSKLTVPKSTW | |
| | QQGWVFSCSVMHEALHNHYTQKSLSLSPGK | |
| 127 | APELLGGPSVFLFPPKPKDTLMISRTPEVTCVVVDVSHEDPEVKFNW | Clone CH3 C.35.18 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVWWESLGHVWVNQQTTPPVLDSDGSFFLYSKLTVPKST | |
| | WQQGWVFSCSVMHEALHNHYTQKSLSLSPGK | |
| 128 | MMDQARSAFSNLFGGEPLSYTRFSLARQVDGDNSHVEMKLGVDEEE | Cyno TfR |
| | NTDNNTKANGTKPKRCGGNICYGTIAVIIFFLIGFMIGYLGYCKGVEP | |
| | KTECERLAGTESPAREEPEEDFPAAPRLYWDDLKRKLSEKLDTTDFT | |
| | STIKLLNENLYVPREAGSQKDENLALYIENQFREFKLSKVWRDQHFV | |
| | KIQVKDSAQNSVIIVDKNGGLVYLVENPGGYVAYSKAATVTGKLVH | |
| | ANFGTKKDFEDLDSPVNGSIVIVRAGKITFAEKVANAESLNAIGVLIY | |
| | MDQTKFPIVKADLSFFGHAHLGTGDPYTPGFPSFNHTQFPPSQSSGLP | |
| | NIPVQTISRAAAEKLFGNMEGDCPSDWKTDSTCKMVTSENKSVKLT | |
| | VSNVLKETKILNIFGVIKGFVEPDHYVVVGAQRDAWGPGAAKSSVG | |
| | TALLLKLAQMFSDMVLKDGFQPSRSIIFASWSAGDFGSVGATEWLEG | |
| | YLSSLHLKAFTYINLDKAVLGTSNFKVSASPLLYTLIEKTMQDVKHP | |
| | VTGRSLYQDSNWASKVEKLTLDNAAFPFLAYSGIPAVSFCFCEDTDY | |
| | PYLGTTMDTYKELVERIPELNKVARAAAEVAGQFVIKLTHDTELNLD | |
| | YERYNSQLLLFLRDLNQYRADVKEMGLSLQWLYSARGDFFRATSRL | |
| | TTDFRNAEKRDKFVMKKLNDRVMRVEYYFLSPYVSPKESPFRHVFW | |
| | GSGSHTLSALLESLKLRRQNNSAFNETLFRNQLALATWTIQGAANAL | |
| | SGDVWDIDNEF | |
| 129 | MGWSCIILFLVATATGAYAGTSSGLPNIPVQTISRAAAEKLFGNMEG | His-tagged permutated |
| | DCPSDWKTDSTCRMVTSESKNVKLTVSNDSAQNSVIIVDKNGRLVY | TfR apical domain |
| | LVENPGGYVAYSKAATVTGKLVHANFGTKKDFEDLYTPVNGSIVIV | |
| | RAGKITFAEKVANAESLNAIGVLIYMDQTKFPIVNAELSASHHHHHH | |
| 130 | METDTLLLWVLLLWVPGSTGDKTHTCPAPEAAGGPSVFLFPPKPKDT | Expressed CH3C.18 Fc |
| | LYITREPEVTCVVVDVSHEDPEVKFNWYVDGVEVHNAKTKPREEQY | sequence |
| | NSTYRVVSVLTVLHQDWLNGKEYKCKVSNKALGAPIEKTISKAKGQ | |
| | PREPQVYTLPPSRDELTKNQVSLTCLVKGFYPSDIAVEWESLGHVWA | |
| | VYKTTPPVLDSDGSFFLYSKLTVPKSTWQQGWVFSCSVMHEALHNH | |
| | YTQKSLSLSPGK | |
| 131 | EWESFGTEWSS | CH3C modified binding |
| | | sequence |
| 132 | EWESYGTEWAS | CH3C modified binding |
| | | sequence |
| 133 | EWESYGTEWVS | CH3C modified binding |
| | | sequence |
| 134 | EWESYGTEWSS | CH3C modified binding |
| | | sequence |
| 135 | EWESFGTEWAS | CH3C modified binding |
| | | sequence |
| 136 | EWESFGTEWVS | CH3C modified binding |
| | | sequence |
| 137 | WWESFGTEWSS | CH3C modified binding |
| | | sequence |
| 138 | WWESYGTEWAS | CH3C modified binding |
| | | sequence |
| 139 | WWESYGTEWVS | CH3C modified binding |
| | | sequence |
| 140 | WWESYGTEWSS | CH3C modified binding |
| | | sequence |
| 141 | WWESFGTEWAS | CH3C modified binding |
| | | sequence |
| 142 | WWESFGTEWVS | CH3C modified binding |
| | | sequence |
| 143 | EWESFGTEWSN | CH3C modified binding |
| | | sequence |
| 144 | EWESYGTEWAN | CH3C modified binding |
| | | sequence |
| 145 | EWESYGTEWVN | CH3C modified binding |
| | | sequence |
| 146 | EWESYGTEWSN | CH3C modified binding |
| | | sequence |
| 147 | EWESFGTEWAN | CH3C modified binding |
| | | sequence |
| 148 | EWESFGTEWVN | CH3C modified binding |
| | | sequence |
| 149 | WWESFGTEWSN | CH3C modified binding |
| | | sequence |
| 150 | WWESYGTEWAN | CH3C modified binding |
| | | sequence |
| 151 | WWESYGTEWVN | CH3C modified binding |
| | | sequence |
| 152 | WWESYGTEWSN | CH3C modified binding |
| | | sequence |
| 153 | WWESFGTEWAN | CH3C modified binding |
| | | sequence |
| 154 | WWESFGTEWVN | CH3C modified binding |
| | | sequence |
| 155 | LWESFGTEWSS | CH3C modified binding |
| | | sequence |
| 156 | LWESYGTEWAS | CH3C modified binding |
| | | sequence |
| 157 | LWESYGTEWVS | CH3C modified binding |
| | | sequence |
| 158 | LWESYGTEWSS | CH3C modified binding |
| | | sequence |
| 159 | LWESFGTEWAS | CH3C modified binding |
| | | sequence |
| 160 | LWESFGTEWVS | CH3C modified binding |
| | | sequence |
| 161 | WWESLGHVWAV | CH3C modified binding |
| | | sequence |
| 162 | EWESLGHVWAV | CH3C modified binding |
| | | sequence |
| 163 | LWESLGHVWAV | CH3C modified binding |
| | | sequence |
| 164 | YWESLGHVWAV | CH3C modified binding |
| | | sequence |
| 165 | EWESLGLVWVF | CH3C modified binding |
| | | sequence |
| 166 | WWESLGHVWVN | CH3C modified binding |
| | | sequence |
| 167 | EWESLGHVWVN | CH3C modified binding |
| | | sequence |
| 168 | TKEEWQQGF | CH3C modified binding |
| | | sequence |
| 169 | SKEEWQQGF | CH3C modified binding |
| | | sequence |
| 170 | PKTSWQQGW | CH3C modified binding |
| | | sequence |
| 171 | TREEWQQGF | CH3C modified binding |
| | | sequence |
| 172 | TPEEWQQGF | CH3C modified binding |
| | | sequence |
| 173 | TGEEWQQGF | CH3C modified binding |
| | | sequence |
| 174 | TVXKXXWQQGXV | Second portion CH3C |
| | | register |
| 175 | APEAAGGPSVFLFPPKPKDTLYITREPEVTCVVVDVSHEDPEVKFNW | Clone CH3C.35.8 |
| | YVDGVEVHNAKTKPREEQYNSTYRVVSVLTVLHQDWLNGKEYKCK | (Clone CH3C.35.20 with |
| | VSNKALGAPIEKTISKAKGQPREPQVYTLPPSRDELTKNQVSLTCLVK | YTE and LALAPG |
| | GFYPSDIAVEWESYGTEWSSYKTTPPVLDSDGSFFLYSKLTVTKEEW | mutations) |
| | QQGFVFSCSVMHEALHNHYTQKSLSLSPGK | |
| 176 | APEAAGGPSVFLFPPKPKDTLYITREPEVTCVVVDVSHEDPEVKFNW | Clone CH3C.35.9 |
| | YVDGVEVHNAKTKPREEQYNSTYRVVSVLTVLHQDWLNGKEYKCK | (Clone CH3C.35.21 with |
| | VSNKALGAPIEKTISKAKGQPREPQVYTLPPSRDELTKNQVSLTCLVK | YTE and LALAPG |
| | GFYPSDIAVEWESFGTEWSSYKTTPPVLDSDGSFFLYSKLTVTKEEW | mutations) |
| | QQGFVFSCSVMHEALHNHYTQKSLSLSPGK | |
| 177 | APELLGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKCK | knob mutation |
| | VSNKALPAPIEKTISKAKGQPREPQVYTLPPSRDELTKNQVSLWCLVK | |
| | GFYPSDIAVEWESFGTEWSSYKTTPPVLDSDGSFFLYSKLTVTKEEW | |
| | QQGFVFSCSVMHEALHNHYTQKSLSLSPGK | |
| 178 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKCK | knob and LALA mutations |
| | VSNKALPAPIEKTISKAKGQPREPQVYTLPPSRDELTKNQVSLWCLVK | |
| | GFYPSDIAVEWESFGTEWSSYKTTPPVLDSDGSFFLYSKLTVTKEEW | |
| | QQGFVFSCSVMHEALHNHYTQKSLSLSPGK | |
| 179 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKCK | knob and LALAPG |
| | VSNKALGAPIEKTISKAKGQPREPQVYTLPPSRDELTKNQVSLWCLV | mutations |
| | KGFYPSDIAVEWESFGTEWSSYKTTPPVLDSDGSFFLYSKLTVTKEE | |
| | WQQGFVFSCSVMHEALHNHYTQKSLSLSPGK | |
| 180 | APELLGGPSVFLFPPKPKDTLYITREPEVTCVVVDVSHEDPEVKFNWY | Clone CH3C.35.20.1 with |
| | VDGVEVHNAKTKPREEQYNSTYRVVSVLTVLHQDWLNGKEYKCKV | knob and YTE mutations |
| | SNKALPAPIEKTISKAKGQPREPQVYTLPPSRDELTKNQVSLWCLVKG | |
| | FYPSDIAVEWESFGTEWSSYKTTPPVLDSDGSFFLYSKLTVTKEEWQ | |
| | QGFVFSCSVMHEALHNHYTQKSLSLSPGK | |
| 181 | APEAAGGPSVFLFPPKPKDTLYITREPEVTCVVVDVSHEDPEVKFNW | Clone CH3C.35.20.1 with |
| | YVDGVEVHNAKTKPREEQYNSTYRVVSVLTVLHQDWLNGKEYKCK | knob, LALA, and YTE |
| | VSNKALPAPIEKTISKAKGQPREPQVYTLPPSRDELTKNQVSLWCLVK | mutations |
| | GFYPSDIAVEWESFGTEWSSYKTTPPVLDSDGSFFLYSKLTVTKEEW | |
| | QQGFVFSCSVMHEALHNHYTQKSLSLSPGK | |
| 182 | APEAAGGPSVFLFPPKPKDTLYITREPEVTCVVVDVSHEDPEVKFNW | Clone CH3C.35.20.1 with |
| | YVDGVEVHNAKTKPREEQYNSTYRVVSVLTVLHQDWLNGKEYKCK | knob, LALAPG, and YTE |
| | VSNKALGAPIEKTISKAKGQPREPQVYTLPPSRDELTKNQVSLWCLV | mutations |
| | KGFYPSDIAVEWESFGTEWSSYKTTPPVLDSDGSFFLYSKLTVTKEE | |
| | WQQGFVFSCSVMHEALHNHYTQKSLSLSPGK | |
| 183 | APELLGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKCK | hole mutations |
| | VSNKALPAPIEKTISKAKGQPREPQVYTLPPSRDELTKNQVSLSCAVK | |
| | GFYPSDIAVEWESFGTEWSSYKTTPPVLDSDGSFFLVSKLTVTKEEW | |
| | QQGFVFSCSVMHEALHNHYTQKSLSLSPGK | |
| 184 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKCK | hole and LALA mutations |
| | VSNKALPAPIEKTISKAKGQPREPQVYTLPPSRDELTKNQVSLSCAVK | |
| | GFYPSDIAVEWESFGTEWSSYKTTPPVLDSDGSFFLVSKLTVTKEEW | |
| | QQGFVFSCSVMHEALHNHYTQKSLSLSPGK | |
| 185 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKCK | hole and LALAPG |
| | VSNKALGAPIEKTISKAKGQPREPQVYTLPPSRDELTKNQVSLSCAVK | mutations |
| | GFYPSDIAVEWESFGTEWSSYKTTPPVLDSDGSFFLVSKLTVTKEEW | |
| | QQGFVFSCSVMHEALHNHYTQKSLSLSPGK | |
| 186 | APELLGGPSVFLFPPKPKDTLYITREPEVTCVVVDVSHEDPEVKFNWY | Clone CH3C.35.20.1 with |
| | VDGVEVHNAKTKPREEQYNSTYRVVSVLTVLHQDWLNGKEYKCKV | hole and YTE mutations |
| | SNKALPAPIEKTISKAKGQPREPQVYTLPPSRDELTKNQVSLSCAVKG | |
| | FYPSDIAVEWESFGTEWSSYKTTPPVLDSDGSFFLVSKLTVTKEEWQ | |
| | QGFVFSCSVMHEALHNHYTQKSLSLSPGK | |
| 187 | APEAAGGPSVFLFPPKPKDTLYITREPEVTCVVVDVSHEDPEVKFNW | Clone CH3C.35.20.1 with |
| | YVDGVEVHNAKTKPREEQYNSTYRVVSVLTVLHQDWLNGKEYKCK | hole, LALA, and YTE |
| | VSNKALPAPIEKTISKAKGQPREPQVYTLPPSRDELTKNQVSLSCAVK | mutations |
| | GFYPSDIAVEWESFGTEWSSYKTTPPVLDSDGSFFLVSKLTVTKEEW | |
| | QQGFVFSCSVMHEALHNHYTQKSLSLSPGK | |
| 188 | APEAAGGPSVFLFPPKPKDTLYITREPEVTCVVVDVSHEDPEVKFNW | Clone CH3C.35.20.1 with |
| | YVDGVEVHNAKTKPREEQYNSTYRVVSVLTVLHQDWLNGKEYKCK | hole, LALAPG, and YTE |
| | VSNKALGAPIEKTISKAKGQPREPQVYTLPPSRDELTKNQVSLSCAVK | mutations |
| | GFYPSDIAVEWESFGTEWSSYKTTPPVLDSDGSFFLVSKLTVTKEEW | |
| | QQGFVFSCSVMHEALHNHYTQKSLSLSPGK | |
| 189 | APELLGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKCK | knob mutation |
| | VSNKALPAPIEKTISKAKGQPREPQVYTLPPSRDELTKNQVSLWCLVK | |
| | GFYPSDIAVEWESYGTEWANYKTTPPVLDSDGSFFLYSKLTVTKEEW | |
| | QQGFVFSCSVMHEALHNHYTQKSLSLSPGK | |
| 190 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKCK | knob and LALA mutations |
| | VSNKALPAPIEKTISKAKGQPREPQVYTLPPSRDELTKNQVSLWCLVK | |
| | GFYPSDIAVEWESYGTEWANYKTTPPVLDSDGSFFLYSKLTVTKEEW | |
| | QQGFVFSCSVMHEALHNHYTQKSLSLSPGK | |
| 191 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKCK | knob and LALAPG |
| | VSNKALGAPIEKTISKAKGQPREPQVYTLPPSRDELTKNQVSLWCLV | mutations |
| | KGFYPSDIAVEWESYGTEWANYKTTPPVLDSDGSFFLYSKLTVTKEE | |
| | WQQGFVFSCSVMHEALHNHYTQKSLSLSPGK | |
| 192 | APELLGGPSVFLFPPKPKDTLYITREPEVTCVVVDVSHEDPEVKFNWY | Clone CH3C.35.23.2 with |
| | VDGVEVHNAKTKPREEQYNSTYRVVSVLTVLHQDWLNGKEYKCKV | knob and YTE mutations |
| | SNKALPAPIEKTISKAKGQPREPQVYTLPPSRDELTKNQVSLWCLVKG | |
| | FYPSDIAVEWESYGTEWANYKTTPPVLDSDGSFFLYSKLTVTKEEWQ | |
| | QGFVFSCSVMHEALHNHYTQKSLSLSPGK | |
| 193 | APEAAGGPSVFLFPPKPKDTLYITREPEVTCVVVDVSHEDPEVKFNW | Clone CH3C.35.23.2 with |
| | YVDGVEVHNAKTKPREEQYNSTYRVVSVLTVLHQDWLNGKEYKCK | knob, LALA, and YTE |
| | VSNKALPAPIEKTISKAKGQPREPQVYTLPPSRDELTKNQVSLWCLVK | mutations |
| | GFYPSDIAVEWESYGTEWANYKTTPPVLDSDGSFFLYSKLTVTKEEW | |
| | QQGFVFSCSVMHEALHNHYTQKSLSLSPGK | |
| 194 | APEAAGGPSVFLFPPKPKDTLYITREPEVTCVVVDVSHEDPEVKFNW | Clone CH3C.35.23.2 with |
| | YVDGVEVHNAKTKPREEQYNSTYRVVSVLTVLHQDWLNGKEYKCK | knob, LALAPG, and YTE |
| | VSNKALGAPIEKTISKAKGQPREPQVYTLPPSRDELTKNQVSLWCLV | mutations |
| | KGFYPSDIAVEWESYGTEWANYKTTPPVLDSDGSFFLYSKLTVTKEE | |
| | WQQGFVFSCSVMHEALHNHYTQKSLSLSPGK | |
| 195 | APELLGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKCK | hole mutations |
| | VSNKALPAPIEKTISKAKGQPREPQVYTLPPSRDELTKNQVSLSCAVK | |
| | GFYPSDIAVEWESYGTEWANYKTTPPVLDSDGSFFLVSKLTVTKEEW | |
| | QQGFVFSCSVMHEALHNHYTQKSLSLSPGK | |
| 196 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKCK | hole and LALA mutations |
| | VSNKALPAPIEKTISKAKGQPREPQVYTLPPSRDELTKNQVSLSCAVK | |
| | GFYPSDIAVEWESYGTEWANYKTTPPVLDSDGSFFLVSKLTVTKEEW | |
| | QQGFVFSCSVMHEALHNHYTQKSLSLSPGK | |
| 197 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKCK | hole and LALAPG |
| | VSNKALGAPIEKTISKAKGQPREPQVYTLPPSRDELTKNQVSLSCAVK | mutations |
| | GFYPSDIAVEWESYGTEWANYKTTPPVLDSDGSFFLVSKLTVTKEEW | |
| | QQGFVFSCSVMHEALHNHYTQKSLSLSPGK | |
| 198 | APELLGGPSVFLFPPKPKDTLYITREPEVTCVVVDVSHEDPEVKFNWY | Clone CH3C.35.23.2 with |
| | VDGVEVHNAKTKPREEQYNSTYRVVSVLTVLHQDWLNGKEYKCKV | hole and YTE mutations |
| | SNKALPAPIEKTISKAKGQPREPQVYTLPPSRDELTKNQVSLSCAVKG | |
| | FYPSDIAVEWESYGTEWANYKTTPPVLDSDGSFFLVSKLTVTKEEWQ | |
| | QGFVFSCSVMHEALHNHYTQKSLSLSPGK | |
| 199 | APEAAGGPSVFLFPPKPKDTLYITREPEVTCVVVDVSHEDPEVKFNW | Clone CH3C.35.23.2 with |
| | YVDGVEVHNAKTKPREEQYNSTYRVVSVLTVLHQDWLNGKEYKCK | hole, LALA, and YTE |
| | VSNKALPAPIEKTISKAKGQPREPQVYTLPPSRDELTKNQVSLSCAVK | mutations |
| | GFYPSDIAVEWESYGTEWANYKTTPPVLDSDGSFFLVSKLTVTKEEW | |
| | QQGFVFSCSVMHEALHNHYTQKSLSLSPGK | |
| 200 | APEAAGGPSVFLFPPKPKDTLYITREPEVTCVVVDVSHEDPEVKFNW | Clone CH3C.35.23.2 with |
| | YVDGVEVHNAKTKPREEQYNSTYRVVSVLTVLHQDWLNGKEYKCK | hole, LALAPG, and YTE |
| | VSNKALGAPIEKTISKAKGQPREPQVYTLPPSRDELTKNQVSLSCAVK | mutations |
| | GFYPSDIAVEWESYGTEWANYKTTPPVLDSDGSFFLVSKLTVTKEEW | |
| | QQGFVFSCSVMHEALHNHYTQKSLSLSPGK | |
| 201 | APELLGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKCK | knob mutation |
| | VSNKALPAPIEKTISKAKGQPREPQVYTLPPSRDELTKNQVSLWCLVK | |
| | GFYPSDIAVEWESYGTEWVNYKTTPPVLDSDGSFFLYSKLTVTKEEW | |
| | QQGFVFSCSVMHEALHNHYTQKSLSLSPGK | |
| 202 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKCK | knob and LALA mutations |
| | VSNKALPAPIEKTISKAKGQPREPQVYTLPPSRDELTKNQVSLWCLVK | |
| | GFYPSDIAVEWESYGTEWVNYKTTPPVLDSDGSFFLYSKLTVTKEEW | |
| | QQGFVFSCSVMHEALHNHYTQKSLSLSPGK | |
| 203 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKCK | knob and LALAPG |
| | VSNKALGAPIEKTISKAKGQPREPQVYTLPPSRDELTKNQVSLWCLV | mutations |
| | KGFYPSDIAVEWESYGTEWVNYKTTPPVLDSDGSFFLYSKLTVTKEE | |
| | WQQGFVFSCSVMHEALHNHYTQKSLSLSPGK | |
| 204 | APELLGGPSVFLFPPKPKDTLYITREPEVTCVVVDVSHEDPEVKFNWY | Clone CH3C.35.23.3 with |
| | VDGVEVHNAKTKPREEQYNSTYRVVSVLTVLHQDWLNGKEYKCKV | knob and YTE mutations |
| | SNKALPAPIEKTISKAKGQPREPQVYTLPPSRDELTKNQVSLWCLVKG | |
| | FYPSDIAVEWESYGTEWVNYKTTPPVLDSDGSFFLYSKLTVTKEEWQ | |
| | QGFVFSCSVMHEALHNHYTQKSLSLSPGK | |
| 205 | APEAAGGPSVFLFPPKPKDTLYITREPEVTCVVVDVSHEDPEVKFNW | Clone CH3C.35.23.3 with |
| | YVDGVEVHNAKTKPREEQYNSTYRVVSVLTVLHQDWLNGKEYKCK | knob, LALA, and YTE |
| | VSNKALPAPIEKTISKAKGQPREPQVYTLPPSRDELTKNQVSLWCLVK | mutations |
| | GFYPSDIAVEWESYGTEWVNYKTTPPVLDSDGSFFLYSKLTVTKEEW | |
| | QQGFVFSCSVMHEALHNHYTQKSLSLSPGK | |
| 206 | APEAAGGPSVFLFPPKPKDTLYITREPEVTCVVVDVSHEDPEVKFNW | Clone CH3C.35.23.3 with |
| | YVDGVEVHNAKTKPREEQYNSTYRVVSVLTVLHQDWLNGKEYKCK | knob, LALAPG, and YTE |
| | VSNKALGAPIEKTISKAKGQPREPQVYTLPPSRDELTKNQVSLWCLV | mutations |
| | KGFYPSDIAVEWESYGTEWVNYKTTPPVLDSDGSFFLYSKLTVTKEE | |
| | WQQGFVFSCSVMHEALHNHYTQKSLSLSPGK | |
| 207 | APELLGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKCK | hole mutations |
| | VSNKALPAPIEKTISKAKGQPREPQVYTLPPSRDELTKNQVSLSCAVK | |
| | GFYPSDIAVEWESYGTEWVNYKTTPPVLDSDGSFFLVSKLTVTKEEW | |
| | QQGFVFSCSVMHEALHNHYTQKSLSLSPGK | |
| 208 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKCK | hole and LALA mutations |
| | VSNKALPAPIEKTISKAKGQPREPQVYTLPPSRDELTKNQVSLSCAVK | |
| | GFYPSDIAVEWESYGTEWVNYKTTPPVLDSDGSFFLVSKLTVTKEEW | |
| | QQGFVFSCSVMHEALHNHYTQKSLSLSPGK | |
| 209 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKCK | hole and LALAPG |
| | VSNKALGAPIEKTISKAKGQPREPQVYTLPPSRDELTKNQVSLSCAVK | mutations |
| | GFYPSDIAVEWESYGTEWVNYKTTPPVLDSDGSFFLVSKLTVTKEEW | |
| | QQGFVFSCSVMHEALHNHYTQKSLSLSPGK | |
| 210 | APELLGGPSVFLFPPKPKDTLYITREPEVTCVVVDVSHEDPEVKFNWY | Clone CH3C.35.23.3 with |
| | VDGVEVHNAKTKPREEQYNSTYRVVSVLTVLHQDWLNGKEYKCKV | hole and YTE mutations |
| | SNKALPAPIEKTISKAKGQPREPQVYTLPPSRDELTKNQVSLSCAVKG | |
| | FYPSDIAVEWESYGTEWVNYKTTPPVLDSDGSFFLVSKLTVTKEEWQ | |
| | QGFVFSCSVMHEALHNHYTQKSLSLSPGK | |
| 211 | APEAAGGPSVFLFPPKPKDTLYITREPEVTCVVVDVSHEDPEVKFNW | Clone CH3C.35.23.3 with |
| | YVDGVEVHNAKTKPREEQYNSTYRVVSVLTVLHQDWLNGKEYKCK | hole, LALA, and YTE |
| | VSNKALPAPIEKTISKAKGQPREPQVYTLPPSRDELTKNQVSLSCAVK | mutations |
| | GFYPSDIAVEWESYGTEWVNYKTTPPVLDSDGSFFLVSKLTVTKEEW | |
| | QQGFVFSCSVMHEALHNHYTQKSLSLSPGK | |
| 212 | APEAAGGPSVFLFPPKPKDTLYITREPEVTCVVVDVSHEDPEVKFNW | Clone CH3C.35.23.3 with |
| | YVDGVEVHNAKTKPREEQYNSTYRVVSVLTVLHQDWLNGKEYKCK | hole, LALAPG, and YTE |
| | VSNKALGAPIEKTISKAKGQPREPQVYTLPPSRDELTKNQVSLSCAVK | mutations |
| | GFYPSDIAVEWESYGTEWVNYKTTPPVLDSDGSFFLVSKLTVTKEEW | |
| | QQGFVFSCSVMHEALHNHYTQKSLSLSPGK | |
| 213 | APELLGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKCK | knob mutation |
| | VSNKALPAPIEKTISKAKGQPREPQVYTLPPSRDELTKNQVSLWCLVK | |
| | GFYPSDIAVEWESYGTEWSNYKTTPPVLDSDGSFFLYSKLTVSKEEW | |
| | QQGFVFSCSVMHEALHNHYTQKSLSLSPGK | |
| 214 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKCK | knob and LALA mutations |
| | VSNKALPAPIEKTISKAKGQPREPQVYTLPPSRDELTKNQVSLWCLVK | |
| | GFYPSDIAVEWESYGTEWSNYKTTPPVLDSDGSFFLYSKLTVSKEEW | |
| | QQGFVFSCSVMHEALHNHYTQKSLSLSPGK | |
| 215 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKCK | knob and LALAPG |
| | VSNKALGAPIEKTISKAKGQPREPQVYTLPPSRDELTKNQVSLWCLV | mutations |
| | KGFYPSDIAVEWESYGTEWSNYKTTPPVLDSDGSFFLYSKLTVSKEE | |
| | WQQGFVFSCSVMHEALHNHYTQKSLSLSPGK | |
| 216 | APELLGGPSVFLFPPKPKDTLYITREPEVTCVVVDVSHEDPEVKFNWY | Clone CH3C.35.23.4 with |
| | VDGVEVHNAKTKPREEQYNSTYRVVSVLTVLHQDWLNGKEYKCKV | knob and YTE mutations |
| | SNKALPAPIEKTISKAKGQPREPQVYTLPPSRDELTKNQVSLWCLVKG | |
| | FYPSDIAVEWESYGTEWSNYKTTPPVLDSDGSFFLYSKLTVSKEEWQ | |
| | QGFVFSCSVMHEALHNHYTQKSLSLSPGK | |
| 217 | APEAAGGPSVFLFPPKPKDTLYITREPEVTCVVVDVSHEDPEVKFNW | Clone CH3C.35.23.4 with |
| | YVDGVEVHNAKTKPREEQYNSTYRVVSVLTVLHQDWLNGKEYKCK | knob, LALA, and YTE |
| | VSNKALPAPIEKTISKAKGQPREPQVYTLPPSRDELTKNQVSLWCLVK | mutations |
| | GFYPSDIAVEWESYGTEWSNYKTTPPVLDSDGSFFLYSKLTVSKEEW | |
| | QQGFVFSCSVMHEALHNHYTQKSLSLSPGK | |
| 218 | APEAAGGPSVFLFPPKPKDTLYITREPEVTCVVVDVSHEDPEVKFNW | Clone CH3C.35.23.4 with |
| | YVDGVEVHNAKTKPREEQYNSTYRVVSVLTVLHQDWLNGKEYKCK | knob, LALAPG, and YTE |
| | VSNKALGAPIEKTISKAKGQPREPQVYTLPPSRDELTKNQVSLWCLV | mutations |
| | KGFYPSDIAVEWESYGTEWSNYKTTPPVLDSDGSFFLYSKLTVSKEE | |
| | WQQGFVFSCSVMHEALHNHYTQKSLSLSPGK | |
| 219 | APELLGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKCK | hole mutations |
| | VSNKALPAPIEKTISKAKGQPREPQVYTLPPSRDELTKNQVSLSCAVK | |
| | GFYPSDIAVEWESYGTEWSNYKTTPPVLDSDGSFFLVSKLTVSKEEW | |
| | QQGFVFSCSVMHEALHNHYTQKSLSLSPGK | |
| 220 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKCK | hole and LALA mutations |
| | VSNKALPAPIEKTISKAKGQPREPQVYTLPPSRDELTKNQVSLSCAVK | |
| | GFYPSDIAVEWESYGTEWSNYKTTPPVLDSDGSFFLVSKLTVSKEEW | |
| | QQGFVFSCSVMHEALHNHYTQKSLSLSPGK | |
| 221 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKCK | hole and LALAPG |
| | VSNKALGAPIEKTISKAKGQPREPQVYTLPPSRDELTKNQVSLSCAVK | mutations |
| | GFYPSDIAVEWESYGTEWSNYKTTPPVLDSDGSFFLVSKLTVSKEEW | |
| | QQGFVFSCSVMHEALHNHYTQKSLSLSPGK | |
| 222 | APELLGGPSVFLFPPKPKDTLYITREPEVTCVVVDVSHEDPEVKFNWY | Clone CH3C.35.23.4 with |
| | VDGVEVHNAKTKPREEQYNSTYRVVSVLTVLHQDWLNGKEYKCKV | hole and YTE mutations |
| | SNKALPAPIEKTISKAKGQPREPQVYTLPPSRDELTKNQVSLSCAVKG | |
| | FYPSDIAVEWESYGTEWSNYKTTPPVLDSDGSFFLVSKLTVSKEEWQ | |
| | QGFVFSCSVMHEALHNHYTQKSLSLSPGK | |
| 223 | APEAAGGPSVFLFPPKPKDTLYITREPEVTCVVVDVSHEDPEVKFNW | Clone CH3C.35.23.4 with |
| | YVDGVEVHNAKTKPREEQYNSTYRVVSVLTVLHQDWLNGKEYKCK | hole, LALA, and YTE |
| | VSNKALPAPIEKTISKAKGQPREPQVYTLPPSRDELTKNQVSLSCAVK | mutations |
| | GFYPSDIAVEWESYGTEWSNYKTTPPVLDSDGSFFLVSKLTVSKEEW | |
| | QQGFVFSCSVMHEALHNHYTQKSLSLSPGK | |
| 224 | APEAAGGPSVFLFPPKPKDTLYITREPEVTCVVVDVSHEDPEVKFNW | Clone CH3C.35.23.4 with |
| | YVDGVEVHNAKTKPREEQYNSTYRVVSVLTVLHQDWLNGKEYKCK | hole, LALAPG, and YTE |
| | VSNKALGAPIEKTISKAKGQPREPQVYTLPPSRDELTKNQVSLSCAVK | mutations |
| | GFYPSDIAVEWESYGTEWSNYKTTPPVLDSDGSFFLVSKLTVSKEEW | |
| | QQGFVFSCSVMHEALHNHYTQKSLSLSPGK | |
| 225 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKCK | with knob mutation |
| | VSNKALPAPIEKTISKAKGQPREPQVYTLPPSRDELTKNQVSLWCLVK | |
| | GFYPSDIAVLWESYGTEWASYKTTPPVLDSDGSFFLYSKLTVTKEEW | |
| | QQGFVFSCSVMHEALHNHYTQKSLSLSPGK | |
| 226 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKCK | with knob and LALA |
| | VSNKALPAPIEKTISKAKGQPREPQVYTLPPSRDELTKNQVSLWCLVK | mutations |
| | GFYPSDIAVLWESYGTEWASYKTTPPVLDSDGSFFLYSKLTVTKEEW | |
| | QQGFVFSCSVMHEALHNHYTQKSLSLSPGK | |
| 227 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKCK | with knob and LALAPG |
| | VSNKALGAPIEKTISKAKGQPREPQVYTLPPSRDELTKNQVSLWCLV | mutations |
| | KGFYPSDIAVLWESYGTEWASYKTTPPVLDSDGSFFLYSKLTVTKEE | |
| | WQQGFVFSCSVMHEALHNHYTQKSLSLSPGK | |
| 228 | APELLGGPSVFLFPPKPKDTLYITREPEVTCVVVDVSHEDPEVKFNWY | Clone CH3C.35.21.17.2 |
| | VDGVEVHNAKTKPREEQYNSTYRVVSVLTVLHQDWLNGKEYKCKV | with knob and YTE |
| | SNKALPAPIEKTISKAKGQPREPQVYTLPPSRDELTKNQVSLWCLVKG | mutations |
| | FYPSDIAVLWESYGTEWASYKTTPPVLDSDGSFFLYSKLTVTKEEWQ | |
| | QGFVFSCSVMHEALHNHYTQKSLSLSPGK | |
| 229 | APEAAGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKCK | with knob, LALA, and |
| | VSNKALPAPIEKTISKAKGQPREPQVYTLPPSRDELTKNQVSLWCLVK | YTE mutations |
| | GFYPSDIAVLWESYGTEWASYKTTPPVLDSDGSFFLYSKLTVTKEEW | |
| | QQGFVFSCSVMHEALHNHYTQKSLSLSPGK | |
| 230 | APEAAGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKCK | with knob, LALAPG, and |
| | VSNKALGAPIEKTISKAKGQPREPQVYTLPPSRDELTKNQVSLWCLV | YTE mutations |
| | KGFYPSDIAVLWESYGTEWASYKTTPPVLDSDGSFFLYSKLTVTKEE | |
| | WQQGFVFSCSVMHEALHNHYTQKSLSLSPGK | |
| 231 | APELLGGPSVFLFPPKPKDTLMISRTPEVTCVVVDVSHEDPEVKFNW | Clone CH3C.3521.17.2 |
| | YVDGVEVHNAKTKPREEQYNSTYRVVSVLTVLHQDWLNGKEYKCK | with hole mutations |
| | VSNKALPAPIEKTISKAKGQPREPQVYTLPPSRDELTKNQVSLSCAVK | |
| | GFYPSDIAVLWESYGTEWASYKTTPPVLDSDGSFFLVSKLTVTKEEW | |
| | QQGFVFSCSVMHEALHNHYTQKSLSLSPGK | |
| 232 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKCK | with hole and LALA |
| | VSNKALPAPIEKTISKAKGQPREPQVYTLPPSRDELTKNQVSLSCAVK | mutations |
| | GFYPSDIAVLWESYGTEWASYKTTPPVLDSDGSFFLVSKLTVTKEEW | |
| | QQGFVFSCSVMHEALHNHYTQKSLSLSPGK | |
| 233 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKCK | with hole and LALAPG |
| | VSNKALGAPIEKTISKAKGQPREPQVYTLPPSRDELTKNQVSLSCAVK | mutations |
| | GFYPSDIAVLWESYGTEWASYKTTPPVLDSDGSFFLVSKLTVTKEEW | |
| | QQGFVFSCSVMHEALHNHYTQKSLSLSPGK | |
| 234 | APELLGGPSVFLFPPKPKDTLYITREPEVTCVVVDVSHEDPEVKFNWY | Clone CH3C.35.21.17.2 |
| | VDGVEVHNAKTKPREEQYNSTYRVVSVLTVLHQDWLNGKEYKCKV | with hole and YTE |
| | SNKALPAPIEKTISKAKGQPREPQVYTLPPSRDELTKNQVSLSCAVKG | mutations |
| | FYPSDIAVLWESYGTEWASYKTTPPVLDSDGSFFLVSKLTVTKEEWQ | |
| | QGFVFSCSVMHEALHNHYTQKSLSLSPGK | |
| 235 | APEAAGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKCK | with hole, LALA, and |
| | VSNKALPAPIEKTISKAKGQPREPQVYTLPPSRDELTKNQVSLSCAVK | YTE mutations |
| | GFYPSDIAVLWESYGTEWASYKTTPPVLDSDGSFFLVSKLTVTKEEW | |
| | QQGFVFSCSVMHEALHNHYTQKSLSLSPGK | |
| 236 | APEAAGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKCK | with hole, LALAPG, and |
| | VSNKALGAPIEKTISKAKGQPREPQVYTLPPSRDELTKNQVSLSCAVK | YTE mutations |
| | GFYPSDIAVLWESYGTEWASYKTTPPVLDSDGSFFLVSKLTVTKEEW | |
| | QQGFVFSCSVMHEALHNHYTQKSLSLSPGK | |
| 237 | APELLGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKCK | knob mutation |
| | VSNKALPAPIEKTISKAKGQPREPQVYTLPPSRDELTKNQVSLWCLVK | |
| | GFYPSDIAVEWESYGTEWSNYKTTPPVLDSDGSFFLYSKLTVTKEEW | |
| | QQGFVFSCSVMHEALHNHYTQKSLSLSPGK | |
| 238 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKCK | knob and LALA mutations |
| | VSNKALPAPIEKTISKAKGQPREPQVYTLPPSRDELTKNQVSLWCLVK | |
| | GFYPSDIAVEWESYGTEWSNYKTTPPVLDSDGSFFLYSKLTVTKEEW | |
| | QQGFVFSCSVMHEALHNHYTQKSLSLSPGK | |
| 239 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKCK | knob and LALAPG |
| | VSNKALGAPIEKTISKAKGQPREPQVYTLPPSRDELTKNQVSLWCLV | mutations |
| | KGFYPSDIAVEWESYGTEWSNYKTTPPVLDSDGSFFLYSKLTVTKEE | |
| | WQQGFVFSCSVMHEALHNHYTQKSLSLSPGK | |
| 240 | APELLGGPSVFLFPPKPKDTLYITREPEVTCVVVDVSHEDPEVKFNWY | Clone CH3C.35.23 with |
| | VDGVEVHNAKTKPREEQYNSTYRVVSVLTVLHQDWLNGKEYKCKV | knob and YTE mutations |
| | SNKALPAPIEKTISKAKGQPREPQVYTLPPSRDELTKNQVSLWCLVKG | |
| | FYPSDIAVEWESYGTEWSNYKTTPPVLDSDGSFFLYSKLTVTKEEWQ | |
| | QGFVFSCSVMHEALHNHYTQKSLSLSPGK | |
| 241 | APEAAGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKCK | knob, LALA, and YTE |
| | VSNKALPAPIEKTISKAKGQPREPQVYTLPPSRDELTKNQVSLWCLVK | mutations |
| | GFYPSDIAVEWESYGTEWSNYKTTPPVLDSDGSFFLYSKLTVTKEEW | |
| | QQGFVFSCSVMHEALHNHYTQKSLSLSPGK | |
| 242 | APEAAGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKCK | knob, LALAPG, and YTE |
| | VSNKALGAPIEKTISKAKGQPREPQVYTLPPSRDELTKNQVSLWCLV | mutations |
| | KGFYPSDIAVEWESYGTEWSNYKTTPPVLDSDGSFFLYSKLTVTKEE | |
| | WQQGFVFSCSVMHEALHNHYTQKSLSLSPGK | |
| 243 | APELLGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKCK | hole mutations |
| | VSNKALPAPIEKTISKAKGQPREPQVYTLPPSRDELTKNQVSLSCAVK | |
| | GFYPSDIAVEWESYGTEWSNYKTTPPVLDSDGSFFLVSKLTVTKEEW | |
| | QQGFVFSCSVMHEALHNHYTQKSLSLSPGK | |
| 244 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKCK | hole and LALA mutations |
| | VSNKALPAPIEKTISKAKGQPREPQVYTLPPSRDELTKNQVSLSCAVK | |
| | GFYPSDIAVEWESYGTEWSNYKTTPPVLDSDGSFFLVSKLTVTKEEW | |
| | QQGFVFSCSVMHEALHNHYTQKSLSLSPGK | |
| 245 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKCK | hole and LALAPG |
| | VSNKALGAPIEKTISKAKGQPREPQVYTLPPSRDELTKNQVSLSCAVK | mutations |
| | GFYPSDIAVEWESYGTEWSNYKTTPPVLDSDGSFFLVSKLTVTKEEW | |
| | QQGFVFSCSVMHEALHNHYTQKSLSLSPGK | |
| 246 | APELLGGPSVFLFPPKPKDTLYITREPEVTCVVVDVSHEDPEVKFNWY | Clone CH3C.35.23 with |
| | VDGVEVHNAKTKPREEQYNSTYRVVSVLTVLHQDWLNGKEYKCKV | hole and YTE mutations |
| | SNKALPAPIEKTISKAKGQPREPQVYTLPPSRDELTKNQVSLSCAVKG | |
| | FYPSDIAVEWESYGTEWSNYKTTPPVLDSDGSFFLVSKLTVTKEEWQ | |
| | QGFVFSCSVMHEALHNHYTQKSLSLSPGK | |
| 247 | APEAAGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKCK | hole, LALA, and YTE |
| | VSNKALPAPIEKTISKAKGQPREPQVYTLPPSRDELTKNQVSLSCAVK | mutations |
| | GFYPSDIAVEWESYGTEWSNYKTTPPVLDSDGSFFLVSKLTVTKEEW | |
| | QQGFVFSCSVMHEALHNHYTQKSLSLSPGK | |
| 248 | APEAAGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKCK | hole, LALAPG, and YTE |
| | VSNKALGAPIEKTISKAKGQPREPQVYTLPPSRDELTKNQVSLSCAVK | mutations |
| | GFYPSDIAVEWESYGTEWSNYKTTPPVLDSDGSFFLVSKLTVTKEEW | |
| | QQGFVFSCSVMHEALHNHYTQKSLSLSPGK | |
| 249 | METDTLLLWVLLLWVPGSTGDKTHTCPPCPAPEAAGGPSVFLFPPKP | Expressed CH3C.35 Fc |
| | KDTLYITREPEVTCVVVDVSHEDPEVKFNWYVDGVEVHNAKTKPRE | sequence |
| | EQYNSTYRVVSVLTVLHQDWLNGKEYKCKVSNKALGAPIEKTISKA | |
| | KGQPREPQVYTLPPSRDELTKNQVSLTCLVKGFYPSDIAVEWESYGT | |
| | EWSSYKTTPPVLDSDGSFFLYSKLTVTKSEWQQGFVFSCSVMHEALH | |
| | NHYTQKSLSLSPGK | |
| 250 | APELLGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKCK | knob |
| | VSNKALPAPIEKTISKAKGQPREPQVYTLPPSRDELTKNQVSLWCLVK | |
| | GFYPSDIAVWWESYGTEWSSYKTTPPVLDSDGSFFLYSKLTVTKEEW | |
| | QQGFVFSCSVMHEALHNHYTQKSLSLSPGK | |
| 251 | APELLGGPSVFLFPPKPKDTLMISRTPEVTCVVVDVSHEDPEVKFNW | Human Fc sequence with |
| | YVDGVEVHNAKTKPREEQYNSTYRVVSVLTVLHQDWLNGKEYKCK | hole mutations |
| | VSNKALPAPIEKTISKAKGQPREPQVYTLPPSRDELTKNQVSLSCAVK | |
| | GFYPSDIAVEWESNGQPENNYKTTPPVLDSDGSFFLVSKLTVDKSRW | |
| | QQGNVFSCSVMHEALHNHYTQKSLSLSPGK | |
| 252 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKCK | knob and LALA mutations |
| | VSNKALPAPIEKTISKAKGQPREPQVYTLPPSRDELTKNQVSLWCLVK | |
| | GFYPSDIAVWWESYGTEWSSYKTTPPVLDSDGSFFLYSKLTVTKEEW | |
| | QQGFVFSCSVMHEALHNHYTQKSLSLSPGK | |
| 253 | APEAAGGPSVFLFPPKPKDTLMISRTPEVTCVVVDVSHEDPEVKFNW | Human Fc sequence with |
| | YVDGVEVHNAKTKPREEQYNSTYRVVSVLTVLHQDWLNGKEYKCK | hole mutations and LALA |
| | VSNKALPAPIEKTISKAKGQPREPQVYTLPPSRDELTKNQVSLSCAVK | mutations |
| | GFYPSDIAVEWESNGQPENNYKTTPPVLDSDGSFFLVSKLTVDKSRW | |
| | QQGNVFSCSVMHEALHNHYTQKSLSLSPGK | |
| 254 | QVQLQQPGAELVKPGASVKMSCKASGYTFTSYNMHWVKQTPGRGL | Heavy chain for anti- |
| | EWIGAIYPGNGDTSYNQKFKGKATLTADKSSSTAYMQLSSLTSEDSA | hCD20-3C.35.21 with |
| | VYYCARSTYYGGDWYFNVWGAGTTVTVSAASTKGPSVFPLAPSSKS | knob mutations |
| | TSGGTAALGCLVKDYFPEPVTVSWNSGALTSGVHTFPAVLQSSGLYS | |
| | LSSVVTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLWCLVK | |
| | GFYPSDIAVWWESYGTEWSSYKTTPPVLDSDGSFFLYSKLTVTKEEW | |
| | QQGFVFSCSVMHEALHNHYTQKSLSLSPGK | |
| 255 | QVQLQQPGAELVKPGASVKMSCKASGYTFTSYNMHWVKQTPGRGL | Heavy chain for anti- |
| | EWIGAIYPGNGDTSYNQKFKGKATLTADKSSSTAYMQLSSLTSEDSA | hCD20-3C.35.21 with |
| | VYYCARSTYYGGDWYFNVWGAGTTVTVSAASTKGPSVFPLAPSSKS | knob and LALA mutations |
| | TSGGTAALGCLVKDYFPEPVTVSWNSGALTSGVHTFPAVLQSSGLYS | |
| | LSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFN | |
| | WYVDGVEVHNAKTKPREEQYNSTYRVVSVLTVLHQDWLNGKEYK | |
| | CKVSNKALPAPIEKTISKAKGQPREPQVYTLPPSRDELTKNQVSLWCL | |
| | VKGFYPSDIAVWWESYGTEWSSYKTTPPVLDSDGSFFLYSKLTVTKE | |
| | EWQQGFVFSCSVMHEALHNHYTQKSLSLSPGK | |
| 256 | QVQLQQPGAELVKPGASVKMSCKASGYTFTSYNMHWVKQTPGRGL | Heavy chain for anti- |
| | EWIGAIYPGNGDTSYNQKFKGKATLTADKSSSTAYMQLSSLTSEDSA | hCD20-Fc with hole |
| | VYYCARSTYYGGDWYFNVWGAGTTVTVSAASTKGPSVFPLAPSSKS | mutations |
| | TSGGTAALGCLVKDYFPEPVTVSWNSGALTSGVHTFPAVLQSSGLYS | |
| | LSSVVTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLSCAVK | |
| | GFYPSDIAVEWESNGQPENNYKTTPPVLDSDGSFFLVSKLTVDKSRW | |
| | QQGNVFSCSVMHEALHNHYTQKSLSLSPGK | |
| 257 | QVQLQQPGAELVKPGASVKMSCKASGYTFTSYNMHWVKQTPGRGL | Heavy chain for anti- |
| | EWIGAIYPGNGDTSYNQKFKGKATLTADKSSSTAYMQLSSLTSEDSA | hCD20-Fc with hole and |
| | VYYCARSTYYGGDWYFNVWGAGTTVTVSAASTKGPSVFPLAPSSKS | LALA mutations |
| | TSGGTAALGCLVKDYFPEPVTVSWNSGALTSGVHTFPAVLQSSGLYS | |
| | LSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFN | |
| | WYVDGVEVHNAKTKPREEQYNSTYRVVSVLTVLHQDWLNGKEYK | |
| | CKVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDKS | |
| | RWQQGNVFSCSVMHEALHNHYTQKSLSLSPGK | |
| 258 | QIVLSQSPAILSASPGEKVTMTCRASSSVSYIHWFQQKPGSSPKPWIY | Light chain for anti- |
| | ATSNLASGVPVRFSGSGSGTSYSLTISRVEAEDAATYYCQQWTSNPPT | hCD20 fusion |
| | FGGGTKLEIKRTVAAPSVFIFPPSDEQLKSGTASVVCLLNNFYPREAK | |
| | VQWKVDNALQSGNSQESVTEQDSKDSTYSLSSTLTLSKADYEKHKV | |
| | YACEVTHQGLSSPVTKSFNRGEC | |
| 259 | QVQLQQPGAELVRPGTSVKLSCKASGYTFTSYWMHWIKQRPGQGLE | Heavy chain for anti- |
| | WIGVIDPSDNYTKYNQKFKGKATLTVDTSSSTAYMQLSSLTSEDSAV | mCD20-3C.35.21 with |
| | YFCAREGYYGSSPWFAYWGQGTLVTVSSASTKGPSVFPLAPSSKSTS | knob mutations |
| | GGTAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPPCP | |
| | APELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLWCLVK | |
| | GFYPSDIAVWWESYGTEWSSYKTTPPVLDSDGSFFLYSKLTVTKEEW | |
| | QQGFVFSCSVMHEALHNHYTQKSLSLSPGK | |
| 260 | QVQLQQPGAELVRPGTSVKLSCKASGYTFTSYWMHWIKQRPGQGLE | Heavy chain for anti- |
| | WIGVIDPSDNYTKYNQKFKGKATLTVDTSSSTAYMQLSSLTSEDSAV | mCD20-3C.35.21 with |
| | YFCAREGYYGSSPWFAYWGQGTLVTVSSASTKGPSVFPLAPSSKSTS | knob and LALA mutations |
| | GGTAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPPCP | |
| | APEAAGGPSVFLFPPKPKDTLMISRTPEVTCVVVDVSHEDPEVKFNW | |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLWCLVK | |
| | GFYPSDIAVWWESYGTEWSSYKTTPPVLDSDGSFFLYSKLTVTKEEW | |
| | QQGFVFSCSVMHEALHNHYTQKSLSLSPGK | |
| 261 | QVQLQQPGAELVRPGTSVKLSCKASGYTFTSYWMHWIKQRPGQGLE | Heavy chain for anti- |
| | WIGVIDPSDNYTKYNQKFKGKATLTVDTSSSTAYMQLSSLTSEDSAV | mCD20-Fc hole |
| | YFCAREGYYGSSPWFAYWGQGTLVTVSSASTKGPSVFPLAPSSKSTS | mutations |
| | GGTAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPPCP | |
| | APELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLSCAVK | |
| | GFYPSDIAVEWESNGQPENNYKTTPPVLDSDGSFFLVSKLTVDKSRW | |
| | QQGNVFSCSVMHEALHNHYTQKSLSLSPGK | |
| 262 | QVQLQQPGAELVRPGTSVKLSCKASGYTFTSYWMHWIKQRPGQGLE | Heavy chain for anti- |
| | WIGVIDPSDNYTKYNQKFKGKATLTVDTSSSTAYMQLSSLTSEDSAV | mCD20-Fc with hole and |
| | YFCAREGYYGSSPWFAYWGQGTLVTVSSASTKGPSVFPLAPSSKSTS | LALA mutations |
| | GGTAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPPCP | |
| | APEAAGGPSVFLFPPKPKDTLMISRTPEVTCVVVDVSHEDPEVKFNW | |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLSCAVK | |
| | GFYPSDIAVEWESNGQPENNYKTTPPVLDSDGSFFLVSKLTVDKSRW | |
| | QQGNVFSCSVMHEALHNHYTQKSLSLSPGK | |
| 263 | QIVMSQSPAILSASPGEKVTMTCRARSSVSYIHWYQQKPGSSPKPWIY | Light chain for anti- |
| | ATSNLASGVPGRFSGSGSGTSYSLTITRVEAEDAATYYCQQWSSKPPT | mCD20 fusion |
| | FGGGTKLEIKRTVAAPSVFIFPPSDEQLKSGTASVVCLLNNFYPREAK | |
| | VQWKVDNALQSGNSQESVTEQDSKDSTYSLSSTLTLSKADYEKHKV | |
| | YACEVTHQGLSSPVTKSFNRGEC | |
| 264 | GAATACATACACTCCTCGTGAGG | sgRNA-1 |
| 265 | AGAAGAATACTTAACATCTTTGG | sgRNA-2 |
| 266 | GCTCAGAACTCCGTGATCATCGTGGATAAGAACGGCCGGCTGGTG | DNA sequence of human |
| | TACCTGGTGGAGAACCCTGGCGGATACGTGGCTTACTCTAAGGCC | apical domain insert |
| | GCTACCGTGACAGGCAAGCTGGTGCACGCCAACTTCGGAACCAAG | |
| | AAGGACTTTGAGGATCTGTACACACCAGTGAACGGCTCTATCGTG | |
| | ATCGTGCGCGCTGGAAAGATCACCTTCGCCGAGAAGGTGGCTAAC | |
| | GCCGAGAGCCTGAACGCCATCGGCGTGCTGATCTACATGGATCAG | |
| | ACAAAGTTTCCCATCGTGAACGCTGAGCTGTCTTTCTTTGGACACG | |
| | CTCACCTGGGCACCGGAGACCCATACACACCCGGATTCCCTAGCT | |
| | TTAACCACACCCAGTTCCCCCCTTCCAGGTCTAGCGGACTGCCAA | |
| | ACATCCCCGTGCAGACAATCAGCAGAGCCGCTGCCGAGAAGCTGT | |
| | TTGGCAACATGGAGGGAGACTGCCCCTCCGATTGGAAGACCGACT | |
| | CTACATGTAGGATGGTGACCTCCGAGTCAAAAAATGTCAAACTCA | |
| | CCGTGTCCAAT | |
| 267 | CTATACAGATATATAAGGATGGGGCTTTTTTTTTTTAATTTTTAAA | Sequence of full donor |
| | AAAGATTTGTTTATTATTATATGTAAGTACACTGTAGCTGTCTTCA | DNA (left homology arm: |
| | GACACTCCAGAAGAGGGCATCAGATCTCATTACAGATGGTTGTGA | 1-817; right homology |
| | GCTACCATGTGGTCACTGGGATTTGAACTCAGGACCTTCAGAAGA | arm: 1523-2329; human |
| | GCAGTCAGTGCTCTTAACTGATAAGTTAATAATAAGTTAACTGAT | apical domain: 941-1492; |
| | AAGGTAATAAAGGTCCCCTATGAAAAGGGTTCAGACCCAAAGAG | codon optimized sequence: |
| | TCAGAGATCCACAGGTTGAGAACCTCCTGCCCTAAATCTTGTTGCT | 821-1522) |
| | CTCCTTATTCAAGACCACTCCTGTTGCAGTTGCTCTTAAGCATGAG | |
| | TATGCTCCCTTCTGAAAGTCTCCATAGCAGCCATCTCTCCAGCCCC | |
| | AGAGTGAGGCTTTTAAAGGAATCTTCATGATAAATAGAATTTTTA | |
| | AAAAAGTAACTGAAGTTACTTAAGGTGTTAAGGTACATTTTATTC | |
| | CCTCAGTAACTGGTTAATCTAGCAGTTTTGAGTCATACTTCATTTA | |
| | TCTTGACTTTGAAGAGTAAGATATTAAAACAATTTGCTTGATCCTT | |
| | GAAGTAAGTATTTAAATAGACATTTTAATGCAGACTTTTTTTAGTT | |
| | GACTGGTGGTGTTGCACGTGGTCAATCCAAGTACTCATGGGAGGC | |
| | AGAGGCAGGAGGATCTCTCTCTAGACCAGCCTGGTCTATAGAGCA | |
| | AGTTCCAGGACAGCCAGGGCTACACAGAAACCTTGTTTCAAACAA | |
| | GACTTTTATCCTTCCAGGCAGCTGAGCCAGAATACATACACTCCT | |
| | AGGGAAGCTGGTTCACAGAAGGACGAATCCCTGGCATACTACATC | |
| | GAGAATCAGTTTCACGAGTTCAAGTTTAGCAAAGTCTGGAGAGAT | |
| | GAGCACTACGTGAAGATCCAGGTGAAGAGCTCCGCTCAGAACTCC | |
| | GTGATCATCGTGGATAAGAACGGCCGGCTGGTGTACCTGGTGGAG | |
| | AACCCTGGCGGATACGTGGCTTACTCTAAGGCCGCTACCGTGACA | |
| | GGCAAGCTGGTGCACGCCAACTTCGGAACCAAGAAGGACTTTGA | |
| | GGATCTGTACACACCAGTGAACGGCTCTATCGTGATCGTGCGCGC | |
| | TGGAAAGATCACCTTCGCCGAGAAGGTGGCTAACGCCGAGAGCCT | |
| | GAACGCCATCGGCGTGCTGATCTACATGGATCAGACAAAGTTTCC | |
| | CATCGTGAACGCTGAGCTGTCTTTCTTTGGACACGCTCACCTGGGC | |
| | ACCGGAGACCCATACACACCCGGATTCCCTAGCTTTAACCACACC | |
| | CAGTTCCCCCCTTCCAGGTCTAGCGGACTGCCAAACATCCCCGTG | |
| | CAGACAATCAGCAGAGCCGCTGCCGAGAAGCTGTTTGGCAACATG | |
| | GAGGGAGACTGCCCCTCCGATTGGAAGACCGACTCTACATGTAGG | |
| | ATGGTGACCTCCGAGTCAAAAAATGTCAAACTCACCGTGTCCAAT | |
| | GTGCTGAAAGAACGACGCATCCTGAATATCTTTGGAGTTATTAAA | |
| | GGTTATGAGGAACCAGGTAAAGACCTGCTTTGTACTTTTTCACTTT | |
| | ACTGTTTTGCTTACTGTAGATAGGTCTAGTGCAGGAAGGAGAAGG | |
| | ATGCTAGCTTGGCATGAACTGCTATATCTTGTTTGTCCTAATGTGA | |
| | ACTTTGTAATATATGTGTATATAACACATAATATGGCCATGTAAGT | |
| | GTATGGAGAGGCCAGAGTTAAGTATTAAATATCTTTCTGTAATCA | |
| | TTTAAAATTTTACATATGAAGGTCAGTGAACAGATTGAAGGAGTT | |
| | TTGTCCAGGTGGGACTTGGATCTAAATTTTTTACAATGCCTGGCAG | |
| | CAAACACCTTTTTAATCAACTGAGCTGTCTCCCCAAATAAAGTGA | |
| | ATGTGATATCAGCTTGTGGATAATTTTTTTTTGTTGCTTTGATAAG | |
| | TGGTTTTCTTACAGGATCACATACCAGTTCTGTCCATAGCATTAAA | |
| | CAAACATAACTGTCATGCAGTAGATTAATGTGCAGGGCACATCCA | |
| | ACAGTCACATTTATTAATAGGACAAAAAGTTGGACCTTATATGTA | |
| | GCACACCTATAATTCCAGTGCTAGGAAGATCCGGGTAGGAGATCC | |
| | TTAGTTCGGTGCTACTTAGTGAGGGTTTGTTTCAAAAAACAAAAG | |
| | CTATGATGGTGTGTTGCCTTTTTTCTTTTAGACCGTTATGTTGTAGT | |
| | AGGAGCCCAGAGAGACGCTTTGGGTGCTGGTGTTGCGGCGAAGTC | |
| | CAGTGTGGGAACAGGTCTTCTGTTGAAACTTGCCCAAGTATTCTC | |
| | AGATATGATTTCAAAAGGT | |
| 268 | APELLGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESFGTEWSSYKTTPPVLDSDGSFFLYSKLTVSKEEW | |
| | QQGFVFSCSVMHEALHNHYTQKSLSLSPGK | |
| 269 | APELLGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWANYKTTPPVLDSDGSFFLYSKLTVSKSEW | |
| | QQGFVFSCSVMHEALHNHYTQKSLSLSPGK | |
| 270 | APELLGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESFGTEWSNYKTTPPVLDSDGSFFLYSKLTVSKEEW | |
| | QQGFVFSCSVMHEALHNHYTQKSLSLSPGK | |
| 271 | APELLGGPSVFLFPPKPKDTLMISRTPEVTCVVVDVSHEDPEVKFNW | Clone CH3C.35.S413 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWSSYKTTPPVLDSDGSFFLYSKLTVSKSEW | |
| | QQGFVFSCSVMHEALHNHYTQKSLSLSPGK | |
| 272 | APELLGGPSVFLFPPKPKDTLMISRTPEVTCVVVDVSHEDPEVKFNW | Clone CH3C.35.23.3.1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWVNYKTTPPVLDSDGSFFLYSKLTVSKEEW | |
| | QQGFVFSCSVMHEALHNHYTQKSLSLSPGK | |
| 273 | APELLGGPSVFLFPPKPKDTLMISRTPEVTCVVVDVSHEDPEVKFNW | Clone CH3C.35.N390.1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESYGTEWSNYKTTPPVLDSDGSFFLYSKLTVSKSEW | |
| | QQGFVFSCSVMHEALHNHYTQKSLSLSPGK | |
| 274 | APELLGGPSVFLFPPKPKDTLMISRTPEVTCVVVDVSHEDPEVKFNW | Clone CH3C.35.23.6.1 |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVK | |
| | GFYPSDIAVEWESFGTEWVNYKTTPPVLDSDGSFFLYSKLTVSKEEW | |
| | QQGFVFSCSVMHEALHNHYTQKSLSLSPGK | |
| 275 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKCK | knob and LALAPG |
| | VSNKALGAPIEKTISKAKGQPREPQVYTLPPSRDELTKNQVSLWCLV | mutations |
| | KGFYPSDIAVWWESYGTEWSSYKTTPPVLDSDGSFFLYSKLTVTKEE | |
| | WQQGFVFSCSVMHEALHNHYTQKSLSLSPGK | |
| 276 | APELLGGPSVFLFPPKPKDTLYITREPEVTCVVVDVSHEDPEVKFNWY | Clone CH3C.35.21 with |
| | VDGVEVHNAKTKPREEQYNSTYRVVSVLTVLHQDWLNGKEYKCKV | knob and YTE mutations |
| | SNKALPAPIEKTISKAKGQPREPQVYTLPPSRDELTKNQVSLWCLVKG | |
| | FYPSDIAVWWESYGTEWSSYKTTPPVLDSDGSFFLYSKLTVTKEEWQ | |
| | QGFVFSCSVMHEALHNHYTQKSLSLSPGK | |
| 277 | APEAAGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKCK | knob, LALA, and YTE |
| | VSNKALPAPIEKTISKAKGQPREPQVYTLPPSRDELTKNQVSLWCLVK | mutations |
| | GFYPSDIAVWWESYGTEWSSYKTTPPVLDSDGSFFLYSKLTVTKEEW | |
| | QQGFVFSCSVMHEALHNHYTQKSLSLSPGK | |
| 278 | APEAAGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKCK | knob, LALAPG, and YTE |
| | VSNKALGAPIEKTISKAKGQPREPQVYTLPPSRDELTKNQVSLWCLV | mutations |
| | KGFYPSDIAVWWESYGTEWSSYKTTPPVLDSDGSFFLYSKLTVTKEE | |
| | WQQGFVFSCSVMHEALHNHYTQKSLSLSPGK | |
| 279 | APELLGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKCK | hole mutations |
| | VSNKALPAPIEKTISKAKGQPREPQVYTLPPSRDELTKNQVSLSCAVK | |
| | GFYPSDIAVWWESYGTEWSSYKTTPPVLDSDGSFFLVSKLTVTKEEW | |
| | QQGFVFSCSVMHEALHNHYTQKSLSLSPGK | |
| 280 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKCK | hole and LALA mutations |
| | VSNKALPAPIEKTISKAKGQPREPQVYTLPPSRDELTKNQVSLSCAVK | |
| | GFYPSDIAVWWESYGTEWSSYKTTPPVLDSDGSFFLVSKLTVTKEEW | |
| | QQGFVFSCSVMHEALHNHYTQKSLSLSPGK | |
| 281 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKCK | hole and LALAPG |
| | VSNKALGAPIEKTISKAKGQPREPQVYTLPPSRDELTKNQVSLSCAVK | mutations |
| | GFYPSDIAVWWESYGTEWSSYKTTPPVLDSDGSFFLVSKLTVTKEEW | |
| | QQGFVFSCSVMHEALHNHYTQKSLSLSPGK | |
| 282 | APELLGGPSVFLFPPKPKDTLYITREPEVTCVVVDVSHEDPEVKFNWY | Clone CH3C.35.21 with |
| | VDGVEVHNAKTKPREEQYNSTYRVVSVLTVLHQDWLNGKEYKCKV | hole and YTE mutations |
| | SNKALPAPIEKTISKAKGQPREPQVYTLPPSRDELTKNQVSLSCAVKG | |
| | FYPSDIAVWWESYGTEWSSYKTTPPVLDSDGSFFLVSKLTVTKEEWQ | |
| | QGFVFSCSVMHEALHNHYTQKSLSLSPGK | |
| 283 | APEAAGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKCK | hole, LALA, and YTE |
| | VSNKALPAPIEKTISKAKGQPREPQVYTLPPSRDELTKNQVSLSCAVK | mutations |
| | GFYPSDIAVWWESYGTEWSSYKTTPPVLDSDGSFFLVSKLTVTKEEW | |
| | QQGFVFSCSVMHEALHNHYTQKSLSLSPGK | |
| 284 | APEAAGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKCK | hole, LALAPG, and YTE |
| | VSNKALGAPIEKTISKAKGQPREPQVYTLPPSRDELTKNQVSLSCAVK | mutations |
| | GFYPSDIAVWWESYGTEWSSYKTTPPVLDSDGSFFLVSKLTVTKEEW | |
| | QQGFVFSCSVMHEALHNHYTQKSLSLSPGK | |
| 285 | APELLGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | with knob mutation |
| | VSNKALPAPIEKTISKAKGQPREPQVYTLPPSRDELTKNQVSLWCLVK | |
| | GFYPSDIAVEWESFGTEWSSYKTTPPVLDSDGSFFLYSKLTVSKEEW | |
| | QQGFVFSCSVMHEALHNHYTQKSLSLSPGK | |
| 286 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | with knob and LALA |
| | VSNKALPAPIEKTISKAKGQPREPQVYTLPPSRDELTKNQVSLWCLVK | mutations |
| | GFYPSDIAVEWESFGTEWSSYKTTPPVLDSDGSFFLYSKLTVSKEEW | |
| | QQGFVFSCSVMHEALHNHYTQKSLSLSPGK | |
| 287 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | with knob and LALAPG |
| | VSNKALGAPIEKTISKAKGQPREPQVYTLPPSRDELTKNQVSLWCLV | mutations |
| | KGFYPSDIAVEWESFGTEWSSYKTTPPVLDSDGSFFLYSKLTVSKEE | |
| | WQQGFVFSCSVMHEALHNHYTQKSLSLSPGK | |
| 288 | APELLGGPSVFLFPPKPKDTLYITREPEVTCVVVDVSHEDPEVKFNWY | Clone CH3C.35.20.1.1 |
| | VDGVEVHNAKTKPREEQYNSTYRVVSVLTVLHQDWLNGKEYKCKV | with knob and YTE |
| | SNKALPAPIEKTISKAKGQPREPQVYTLPPSRDELTKNQVSLWCLVKG | mutations |
| | FYPSDIAVEWESFGTEWSSYKTTPPVLDSDGSFFLYSKLTVSKEEWQ | |
| | QGFVFSCSVMHEALHNHYTQKSLSLSPGK | |
| 289 | APEAAGGPSVFLFPPKPKDTLYITREPEVTCVVVDVSHEDPEVKFNW | Clone CH3C.35.20.1.1 |
| | YVDGVEVHNAKTKPREEQYNSTYRVVSVLTVLHQDWLNGKEYKCK | with knob, LALA, and |
| | VSNKALPAPIEKTISKAKGQPREPQVYTLPPSRDELTKNQVSLWCLVK | YTE mutations |
| | GFYPSDIAVEWESFGTEWSSYKTTPPVLDSDGSFFLYSKLTVSKEEW | |
| | QQGFVFSCSVMHEALHNHYTQKSLSLSPGK | |
| 290 | APEAAGGPSVFLFPPKPKDTLYITREPEVTCVVVDVSHEDPEVKFNW | Clone CH3C.35.20.1.1 |
| | YVDGVEVHNAKTKPREEQYNSTYRVVSVLTVLHQDWLNGKEYKCK | with knob, LALAPG, and |
| | VSNKALGAPIEKTISKAKGQPREPQVYTLPPSRDELTKNQVSLWCLV | YTE mutations |
| | KGFYPSDIAVEWESFGTEWSSYKTTPPVLDSDGSFFLYSKLTVSKEE | |
| | WQQGFVFSCSVMHEALHNHYTQKSLSLSPGK | |
| 291 | APELLGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | with hole mutations |
| | VSNKALPAPIEKTISKAKGQPREPQVYTLPPSRDELTKNQVSLSCAVK | |
| | GFYPSDIAVEWESFGTEWSSYKTTPPVLDSDGSFFLVSKLTVSKEEW | |
| | QQGFVFSCSVMHEALHNHYTQKSLSLSPGK | |
| 292 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | with hole and LALA |
| | VSNKALPAPIEKTISKAKGQPREPQVYTLPPSRDELTKNQVSLSCAVK | mutations |
| | GFYPSDIAVEWESFGTEWSSYKTTPPVLDSDGSFFLVSKLTVSKEEW | |
| | QQGFVFSCSVMHEALHNHYTQKSLSLSPGK | |
| 293 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | with hole and LALAPG |
| | VSNKALGAPIEKTISKAKGQPREPQVYTLPPSRDELTKNQVSLSCAVK | mutations |
| | GFYPSDIAVEWESFGTEWSSYKTTPPVLDSDGSFFLVSKLTVSKEEW | |
| | QQGFVFSCSVMHEALHNHYTQKSLSLSPGK | |
| 294 | APELLGGPSVFLFPPKPKDTLYITREPEVTCVVVDVSHEDPEVKFNWY | Clone CH3C.35.20.1.1 |
| | VDGVEVHNAKTKPREEQYNSTYRVVSVLTVLHQDWLNGKEYKCKV | with hole and YTE |
| | SNKALPAPIEKTISKAKGQPREPQVYTLPPSRDELTKNQVSLSCAVKG | mutations |
| | FYPSDIAVEWESFGTEWSSYKTTPPVLDSDGSFFLVSKLTVSKEEWQ | |
| | QGFVFSCSVMHEALHNHYTQKSLSLSPGK | |
| 295 | APEAAGGPSVFLFPPKPKDTLYITREPEVTCVVVDVSHEDPEVKFNW | Clone CH3C.35.20.1.1 |
| | YVDGVEVHNAKTKPREEQYNSTYRVVSVLTVLHQDWLNGKEYKCK | with hole, LALA, and |
| | VSNKALPAPIEKTISKAKGQPREPQVYTLPPSRDELTKNQVSLSCAVK | YTE mutations |
| | GFYPSDIAVEWESFGTEWSSYKTTPPVLDSDGSFFLVSKLTVSKEEW | |
| | QQGFVFSCSVMHEALHNHYTQKSLSLSPGK | |
| 296 | APEAAGGPSVFLFPPKPKDTLYITREPEVTCVVVDVSHEDPEVKFNW | Clone CH3C.35.20.1.1 |
| | YVDGVEVHNAKTKPREEQYNSTYRVVSVLTVLHQDWLNGKEYKCK | with hole, LALAPG, and |
| | VSNKALGAPIEKTISKAKGQPREPQVYTLPPSRDELTKNQVSLSCAVK | YTE mutations |
| | GFYPSDIAVEWESFGTEWSSYKTTPPVLDSDGSFFLVSKLTVSKEEW | |
| | QQGFVFSCSVMHEALHNHYTQKSLSLSPGK | |
| 297 | APELLGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | with knob mutation |
| | VSNKALPAPIEKTISKAKGQPREPQVYTLPPSRDELTKNQVSLWCLVK | |
| | GFYPSDIAVEWESYGTEWANYKTTPPVLDSDGSFFLYSKLTVSKSEW | |
| | QQGFVFSCSVMHEALHNHYTQKSLSLSPGK | |
| 298 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | with knob and LALA |
| | VSNKALPAPIEKTISKAKGQPREPQVYTLPPSRDELTKNQVSLWCLVK | mutations |
| | GFYPSDIAVEWESYGTEWANYKTTPPVLDSDGSFFLYSKLTVSKSEW | |
| | QQGFVFSCSVMHEALHNHYTQKSLSLSPGK | |
| 299 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | with knob and LALAPG |
| | VSNKALGAPIEKTISKAKGQPREPQVYTLPPSRDELTKNQVSLWCLV | mutations |
| | KGFYPSDIAVEWESYGTEWANYKTTPPVLDSDGSFFLYSKLTVSKSE | |
| | WQQGFVFSCSVMHEALHNHYTQKSLSLSPGK | |
| 300 | APELLGGPSVFLFPPKPKDTLYITREPEVTCVVVDVSHEDPEVKFNWY | Clone CH3C.35.23.2.1 |
| | VDGVEVHNAKTKPREEQYNSTYRVVSVLTVLHQDWLNGKEYKCKV | with knob and YTE |
| | SNKALPAPIEKTISKAKGQPREPQVYTLPPSRDELTKNQVSLWCLVKG | mutations |
| | FYPSDIAVEWESYGTEWANYKTTPPVLDSDGSFFLYSKLTVSKSEWQ | |
| | QGFVFSCSVMHEALHNHYTQKSLSLSPGK | |
| 301 | APEAAGGPSVFLFPPKPKDTLYITREPEVTCVVVDVSHEDPEVKFNW | Clone CH3C.35.23.2.1 |
| | YVDGVEVHNAKTKPREEQYNSTYRVVSVLTVLHQDWLNGKEYKCK | with knob, LALA, and |
| | VSNKALPAPIEKTISKAKGQPREPQVYTLPPSRDELTKNQVSLWCLVK | YTE mutations |
| | GFYPSDIAVEWESYGTEWANYKTTPPVLDSDGSFFLYSKLTVSKSEW | |
| | QQGFVFSCSVMHEALHNHYTQKSLSLSPGK | |
| 302 | APEAAGGPSVFLFPPKPKDTLYITREPEVTCVVVDVSHEDPEVKFNW | Clone CH3C.35.23.2.1 |
| | YVDGVEVHNAKTKPREEQYNSTYRVVSVLTVLHQDWLNGKEYKCK | with knob, LALAPG, and |
| | VSNKALGAPIEKTISKAKGQPREPQVYTLPPSRDELTKNQVSLWCLV | YTE mutations |
| | KGFYPSDIAVEWESYGTEWANYKTTPPVLDSDGSFFLYSKLTVSKSE | |
| | WQQGFVFSCSVMHEALHNHYTQKSLSLSPGK | |
| 303 | APELLGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | with hole mutations |
| | VSNKALPAPIEKTISKAKGQPREPQVYTLPPSRDELTKNQVSLSCAVK | |
| | GFYPSDIAVEWESYGTEWANYKTTPPVLDSDGSFFLVSKLTVSKSEW | |
| | QQGFVFSCSVMHEALHNHYTQKSLSLSPGK | |
| 304 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | with hole and LALA |
| | VSNKALPAPIEKTISKAKGQPREPQVYTLPPSRDELTKNQVSLSCAVK | mutations |
| | GFYPSDIAVEWESYGTEWANYKTTPPVLDSDGSFFLVSKLTVSKSEW | |
| | QQGFVFSCSVMHEALHNHYTQKSLSLSPGK | |
| 305 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | with hole and LALAPG |
| | VSNKALGAPIEKTISKAKGQPREPQVYTLPPSRDELTKNQVSLSCAVK | mutations |
| | GFYPSDIAVEWESYGTEWANYKTTPPVLDSDGSFFLVSKLTVSKSEW | |
| | QQGFVFSCSVMHEALHNHYTQKSLSLSPGK | |
| 306 | APELLGGPSVFLFPPKPKDTLYITREPEVTCVVVDVSHEDPEVKFNWY | Clone CH3C.35.23.2.1 |
| | VDGVEVHNAKTKPREEQYNSTYRVVSVLTVLHQDWLNGKEYKCKV | with hole and YTE |
| | SNKALPAPIEKTISKAKGQPREPQVYTLPPSRDELTKNQVSLSCAVKG | mutations |
| | FYPSDIAVEWESYGTEWANYKTTPPVLDSDGSFFLVSKLTVSKSEWQ | |
| | QGFVFSCSVMHEALHNHYTQKSLSLSPGK | |
| 307 | APEAAGGPSVFLFPPKPKDTLYITREPEVTCVVVDVSHEDPEVKFNW | Clone CH3C.35.23.2.1 |
| | YVDGVEVHNAKTKPREEQYNSTYRVVSVLTVLHQDWLNGKEYKCK | with hole, LALA, and |
| | VSNKALPAPIEKTISKAKGQPREPQVYTLPPSRDELTKNQVSLSCAVK | YTE mutations |
| | GFYPSDIAVEWESYGTEWANYKTTPPVLDSDGSFFLVSKLTVSKSEW | |
| | QQGFVFSCSVMHEALHNHYTQKSLSLSPGK | |
| 308 | APEAAGGPSVFLFPPKPKDTLYITREPEVTCVVVDVSHEDPEVKFNW | Clone CH3C.35.23.2.1 |
| | YVDGVEVHNAKTKPREEQYNSTYRVVSVLTVLHQDWLNGKEYKCK | with hole, LALAPG, and |
| | VSNKALGAPIEKTISKAKGQPREPQVYTLPPSRDELTKNQVSLSCAVK | YTE mutations |
| | GFYPSDIAVEWESYGTEWANYKTTPPVLDSDGSFFLVSKLTVSKSEW | |
| | QQGFVFSCSVMHEALHNHYTQKSLSLSPGK | |
| 309 | APELLGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKCK | with knob mutation |
| | VSNKALPAPIEKTISKAKGQPREPQVYTLPPSRDELTKNQVSLWCLVK | |
| | GFYPSDIAVEWESFGTEWSNYKTTPPVLDSDGSFFLYSKLTVSKEEW | |
| | QQGFVFSCSVMHEALHNHYTQKSLSLSPGK | |
| 310 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKCK | with knob and LALA |
| | VSNKALPAPIEKTISKAKGQPREPQVYTLPPSRDELTKNQVSLWCLVK | mutations |
| | GFYPSDIAVEWESFGTEWSNYKTTPPVLDSDGSFFLYSKLTVSKEEW | |
| | QQGFVFSCSVMHEALHNHYTQKSLSLSPGK | |
| 311 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKCK | with knob and LALAPG |
| | VSNKALGAPIEKTISKAKGQPREPQVYTLPPSRDELTKNQVSLWCLV | mutations |
| | KGFYPSDIAVEWESFGTEWSNYKTTPPVLDSDGSFFLYSKLTVSKEE | |
| | WQQGFVFSCSVMHEALHNHYTQKSLSLSPGK | |
| 312 | APELLGGPSVFLFPPKPKDTLYITREPEVTCVVVDVSHEDPEVKFNWY | Clone CH3C.35.23.1.1 |
| | VDGVEVHNAKTKPREEQYNSTYRVVSVLTVLHQDWLNGKEYKCKV | with knob and YTE |
| | SNKALPAPIEKTISKAKGQPREPQVYTLPPSRDELTKNQVSLWCLVKG | mutations |
| | FYPSDIAVEWESFGTEWSNYKTTPPVLDSDGSFFLYSKLTVSKEEWQ | |
| | QGFVFSCSVMHEALHNHYTQKSLSLSPGK | |
| 313 | APEAAGGPSVFLFPPKPKDTLYITREPEVTCVVVDVSHEDPEVKFNW | Clone CH3C.35.23.1.1 |
| | YVDGVEVHNAKTKPREEQYNSTYRVVSVLTVLHQDWLNGKEYKCK | with knob, LALA, and |
| | VSNKALPAPIEKTISKAKGQPREPQVYTLPPSRDELTKNQVSLWCLVK | YTE mutations |
| | GFYPSDIAVEWESFGTEWSNYKTTPPVLDSDGSFFLYSKLTVSKEEW | |
| | QQGFVFSCSVMHEALHNHYTQKSLSLSPGK | |
| 314 | APEAAGGPSVFLFPPKPKDTLYITREPEVTCVVVDVSHEDPEVKFNW | Clone CH3C.35.23.1.1 |
| | YVDGVEVHNAKTKPREEQYNSTYRVVSVLTVLHQDWLNGKEYKCK | with knob, LALAPG, and |
| | VSNKALGAPIEKTISKAKGQPREPQVYTLPPSRDELTKNQVSLWCLV | YTE mutations |
| | KGFYPSDIAVEWESFGTEWSNYKTTPPVLDSDGSFFLYSKLTVSKEE | |
| | WQQGFVFSCSVMHEALHNHYTQKSLSLSPGK | |
| 315 | APELLGGPSVFLFPPKPKDTLMISRTPEVTCVVVDVSHEDPEVKFNW | Clone CH3C.3523.1.1 |
| | YVDGVEVHNAKTKPREEQYNSTYRVVSVLTVLHQDWLNGKEYKCK | with hole mutations |
| | VSNKALPAPIEKTISKAKGQPREPQVYTLPPSRDELTKNQVSLSCAVK | |
| | GFYPSDIAVEWESFGTEWSNYKTTPPVLDSDGSFFLVSKLTVSKEEW | |
| | QQGFVFSCSVMHEALHNHYTQKSLSLSPGK | |
| 316 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKCK | with hole and LALA |
| | VSNKALPAPIEKTISKAKGQPREPQVYTLPPSRDELTKNQVSLSCAVK | mutations |
| | GFYPSDIAVEWESFGTEWSNYKTTPPVLDSDGSFFLVSKLTVSKEEW | |
| | QQGFVFSCSVMHEALHNHYTQKSLSLSPGK | |
| 317 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKCK | with hole and LALAPG |
| | VSNKALGAPIEKTISKAKGQPREPQVYTLPPSRDELTKNQVSLSCAVK | mutations |
| | GFYPSDIAVEWESFGTEWSNYKTTPPVLDSDGSFFLVSKLTVSKEEW | |
| | QQGFVFSCSVMHEALHNHYTQKSLSLSPGK | |
| 318 | APELLGGPSVFLFPPKPKDTLYITREPEVTCVVVDVSHEDPEVKFNWY | Clone CH3C.35.23.1.1 |
| | VDGVEVHNAKTKPREEQYNSTYRVVSVLTVLHQDWLNGKEYKCKV | with hole and YTE |
| | SNKALPAPIEKTISKAKGQPREPQVYTLPPSRDELTKNQVSLSCAVKG | mutations |
| | FYPSDIAVEWESFGTEWSNYKTTPPVLDSDGSFFLVSKLTVSKEEWQ | |
| | QGFVFSCSVMHEALHNHYTQKSLSLSPGK | |
| 319 | APEAAGGPSVFLFPPKPKDTLYITREPEVTCVVVDVSHEDPEVKFNW | Clone CH3C.35.23.1.1 |
| | YVDGVEVHNAKTKPREEQYNSTYRVVSVLTVLHQDWLNGKEYKCK | with hole, LALA, and |
| | VSNKALPAPIEKTISKAKGQPREPQVYTLPPSRDELTKNQVSLSCAVK | YTE mutations |
| | GFYPSDIAVEWESFGTEWSNYKTTPPVLDSDGSFFLVSKLTVSKEEW | |
| | QQGFVFSCSVMHEALHNHYTQKSLSLSPGK | |
| 320 | APEAAGGPSVFLFPPKPKDTLYITREPEVTCVVVDVSHEDPEVKFNW | Clone CH3C.35.23.1.1 |
| | YVDGVEVHNAKTKPREEQYNSTYRVVSVLTVLHQDWLNGKEYKCK | with hole, LALAPG, and |
| | VSNKALGAPIEKTISKAKGQPREPQVYTLPPSRDELTKNQVSLSCAVK | YTE mutations |
| | GFYPSDIAVEWESFGTEWSNYKTTPPVLDSDGSFFLVSKLTVSKEEW | |
| | QQGFVFSCSVMHEALHNHYTQKSLSLSPGK | |
| 321 | APELLGGPSVFLFPPKPKDTLMISRTPEVTCVVVDVSHEDPEVKFNW | Clone CH3C.35.20.1 |
| | YVDGVEVHNAKTKPREEQYNSTYRVVSVLTVLHQDWLNGKEYKCK | M198L and N204S |
| | VSNKALPAPIEKTISKAKGQPREPQVYTLPPSRDELTKNQVSLTCLVK | mutations |
| | GFYPSDIAVEWESFGTEWSSYKTTPPVLDSDGSFFLYSKLTVTKEEW | |
| | QQGFVFSCSVLHEALHSHYTQKSLSLSPGK | |
| 322 | APELLGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKCK | knob and M198L and |
| | VSNKALPAPIEKTISKAKGQPREPQVYTLPPSRDELTKNQVSLWCLVK | N204S mutations |
| | GFYPSDIAVEWESFGTEWSSYKTTPPVLDSDGSFFLYSKLTVTKEEW | |
| | QQGFVFSCSVLHEALHSHYTQKSLSLSPGK | |
| 323 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKCK | knob, LALA, and M198L |
| | VSNKALPAPIEKTISKAKGQPREPQVYTLPPSRDELTKNQVSLWCLVK | and N204S mutations |
| | GFYPSDIAVEWESFGTEWSSYKTTPPVLDSDGSFFLYSKLTVTKEEW | |
| | QQGFVFSCSVLHEALHSHYTQKSLSLSPGK | |
| 324 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKCK | knob LALAPG and |
| | VSNKALGAPIEKTISKAKGQPREPQVYTLPPSRDELTKNQVSLWCLV | M198L and N204S |
| | KGFYPSDIAVEWESFGTEWSSYKTTPPVLDSDGSFFLYSKLTVTKEE | mutations |
| | WQQGFVFSCSVLHEALHSHYTQKSLSLSPGK | |
| 325 | APELLGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKCK | hole and M198L and |
| | VSNKALPAPIEKTISKAKGQPREPQVYTLPPSRDELTKNQVSLSCAVK | N204S mutations |
| | GFYPSDIAVEWESFGTEWSSYKTTPPVLDSDGSFFLVSKLTVTKEEW | |
| | QQGFVFSCSVLHEALHSHYTQKSLSLSPGK | |
| 326 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKCK | hole, LALA, and M198L |
| | VSNKALPAPIEKTISKAKGQPREPQVYTLPPSRDELTKNQVSLSCAVK | and N204S mutations |
| | GFYPSDIAVEWESFGTEWSSYKTTPPVLDSDGSFFLVSKLTVTKEEW | |
| | QQGFVFSCSVLHEALHSHYTQKSLSLSPGK | |
| 327 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKCK | hole LALAPG and |
| | VSNKALGAPIEKTISKAKGQPREPQVYTLPPSRDELTKNQVSLSCAVK | M198L and N204S |
| | GFYPSDIAVEWESFGTEWSSYKTTPPVLDSDGSFFLVSKLTVTKEEW | mutations |
| | QQGFVFSCSVLHEALHSHYTQKSLSLSPGK | |
| 328 | APELLGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKCK | M198L and N204S |
| | VSNKALPAPIEKTISKAKGQPREPQVYTLPPSRDELTKNQVSLTCLVK | mutations |
| | GFYPSDIAVEWESYGTEWANYKTTPPVLDSDGSFFLYSKLTVTKEEW | |
| | QQGFVFSCSVLHEALHSHYTQKSLSLSPGK | |
| 329 | APELLGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKCK | knob and M198L and |
| | VSNKALPAPIEKTISKAKGQPREPQVYTLPPSRDELTKNQVSLWCLVK | N204S mutations |
| | GFYPSDIAVEWESYGTEWANYKTTPPVLDSDGSFFLYSKLTVTKEEW | |
| | QQGFVFSCSVLHEALHSHYTQKSLSLSPGK | |
| 330 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKCK | knob, LALA, and M198L |
| | VSNKALPAPIEKTISKAKGQPREPQVYTLPPSRDELTKNQVSLWCLVK | and N204S mutations |
| | GFYPSDIAVEWESYGTEWANYKTTPPVLDSDGSFFLYSKLTVTKEEW | |
| | QQGFVFSCSVLHEALHSHYTQKSLSLSPGK | |
| 331 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKCK | knob LALAPG and |
| | VSNKALGAPIEKTISKAKGQPREPQVYTLPPSRDELTKNQVSLWCLV | M198L and N204S |
| | KGFYPSDIAVEWESYGTEWANYKTTPPVLDSDGSFFLYSKLTVTKEE | mutations |
| | WQQGFVFSCSVLHEALHSHYTQKSLSLSPGK | |
| 332 | APELLGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKCK | hole and M198L and |
| | VSNKALPAPIEKTISKAKGQPREPQVYTLPPSRDELTKNQVSLSCAVK | N204S mutations |
| | GFYPSDIAVEWESYGTEWANYKTTPPVLDSDGSFFLVSKLTVTKEEW | |
| | QQGFVFSCSVLHEALHSHYTQKSLSLSPGK | |
| 333 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKCK | hole, LALA, and M198L |
| | VSNKALPAPIEKTISKAKGQPREPQVYTLPPSRDELTKNQVSLSCAVK | and N204S mutations |
| | GFYPSDIAVEWESYGTEWANYKTTPPVLDSDGSFFLVSKLTVTKEEW | |
| | QQGFVFSCSVLHEALHSHYTQKSLSLSPGK | |
| 334 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKCK | hole, LALAPG, and |
| | VSNKALGAPIEKTISKAKGQPREPQVYTLPPSRDELTKNQVSLSCAVK | M198L and N204S |
| | GFYPSDIAVEWESYGTEWANYKTTPPVLDSDGSFFLVSKLTVTKEEW | mutations |
| | QQGFVFSCSVLHEALHSHYTQKSLSLSPGK | |
| 335 | APELLGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKCK | M198L and N204S |
| | VSNKALPAPIEKTISKAKGQPREPQVYTLPPSRDELTKNQVSLTCLVK | mutations |
| | GFYPSDIAVEWESYGTEWVNYKTTPPVLDSDGSFFLYSKLTVTKEEW | |
| | QQGFVFSCSVLHEALHSHYTQKSLSLSPGK | |
| 336 | APELLGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKCK | knob and M198L and |
| | VSNKALPAPIEKTISKAKGQPREPQVYTLPPSRDELTKNQVSLWCLVK | N204S mutations |
| | GFYPSDIAVEWESYGTEWVNYKTTPPVLDSDGSFFLYSKLTVTKEEW | |
| | QQGFVFSCSVLHEALHSHYTQKSLSLSPGK | |
| 337 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKCK | knob, LALA, and M198L |
| | VSNKALPAPIEKTISKAKGQPREPQVYTLPPSRDELTKNQVSLWCLVK | and N204S mutations |
| | GFYPSDIAVEWESYGTEWVNYKTTPPVLDSDGSFFLYSKLTVTKEEW | |
| | QQGFVFSCSVLHEALHSHYTQKSLSLSPGK | |
| 338 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKCK | knob, LALAPG, and |
| | VSNKALGAPIEKTISKAKGQPREPQVYTLPPSRDELTKNQVSLWCLV | M198L and N204S |
| | KGFYPSDIAVEWESYGTEWVNYKTTPPVLDSDGSFFLYSKLTVTKEE | mutations |
| | WQQGFVFSCSVLHEALHSHYTQKSLSLSPGK | |
| 339 | APELLGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKCK | hole and M198L and |
| | VSNKALPAPIEKTISKAKGQPREPQVYTLPPSRDELTKNQVSLSCAVK | N204S mutations |
| | GFYPSDIAVEWESYGTEWVNYKTTPPVLDSDGSFFLVSKLTVTKEEW | |
| | QQGFVFSCSVLHEALHSHYTQKSLSLSPGK | |
| 340 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKCK | hole, LALA, and M198L |
| | VSNKALPAPIEKTISKAKGQPREPQVYTLPPSRDELTKNQVSLSCAVK | and N204S mutations |
| | GFYPSDIAVEWESYGTEWVNYKTTPPVLDSDGSFFLVSKLTVTKEEW | |
| | QQGFVFSCSVLHEALHSHYTQKSLSLSPGK | |
| 341 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKCK | hole, LALAPG, and |
| | VSNKALGAPIEKTISKAKGQPREPQVYTLPPSRDELTKNQVSLSCAVK | M198L and N204S |
| | GFYPSDIAVEWESYGTEWVNYKTTPPVLDSDGSFFLVSKLTVTKEEW | mutations |
| | QQGFVFSCSVLHEALHSHYTQKSLSLSPGK | |
| 342 | APELLGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKCK | M198L and N204S |
| | VSNKALPAPIEKTISKAKGQPREPQVYTLPPSRDELTKNQVSLTCLVK | mutations |
| | GFYPSDIAVEWESYGTEWSNYKTTPPVLDSDGSFFLYSKLTVSKEEW | |
| | QQGFVFSCSVLHEALHSHYTQKSLSLSPGK | |
| 343 | APELLGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKCK | knob and M198L and |
| | VSNKALPAPIEKTISKAKGQPREPQVYTLPPSRDELTKNQVSLWCLVK | N204S mutations |
| | GFYPSDIAVEWESYGTEWSNYKTTPPVLDSDGSFFLYSKLTVSKEEW | |
| | QQGFVFSCSVLHEALHSHYTQKSLSLSPGK | |
| 344 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKCK | knob, LALA, and M198L |
| | VSNKALPAPIEKTISKAKGQPREPQVYTLPPSRDELTKNQVSLWCLVK | and N204S mutations |
| | GFYPSDIAVEWESYGTEWSNYKTTPPVLDSDGSFFLYSKLTVSKEEW | |
| | QQGFVFSCSVLHEALHSHYTQKSLSLSPGK | |
| 345 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKCK | knob, LALAPG, and |
| | VSNKALGAPIEKTISKAKGQPREPQVYTLPPSRDELTKNQVSLWCLV | M198L and N204S |
| | KGFYPSDIAVEWESYGTEWSNYKTTPPVLDSDGSFFLYSKLTVSKEE | mutations |
| | WQQGFVFSCSVLHEALHSHYTQKSLSLSPGK | |
| 346 | APELLGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKCK | hole and M198L and |
| | VSNKALPAPIEKTISKAKGQPREPQVYTLPPSRDELTKNQVSLSCAVK | N204S mutations |
| | GFYPSDIAVEWESYGTEWSNYKTTPPVLDSDGSFFLVSKLTVSKEEW | |
| | QQGFVFSCSVLHEALHSHYTQKSLSLSPGK | |
| 347 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKCK | hole, LALA, and M198L |
| | VSNKALPAPIEKTISKAKGQPREPQVYTLPPSRDELTKNQVSLSCAVK | and N204S mutations |
| | GFYPSDIAVEWESYGTEWSNYKTTPPVLDSDGSFFLVSKLTVSKEEW | |
| | QQGFVFSCSVLHEALHSHYTQKSLSLSPGK | |
| 348 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKCK | hole, LALAPG, and |
| | VSNKALGAPIEKTISKAKGQPREPQVYTLPPSRDELTKNQVSLSCAVK | M198L and N204S |
| | GFYPSDIAVEWESYGTEWSNYKTTPPVLDSDGSFFLVSKLTVSKEEW | mutations |
| | QQGFVFSCSVLHEALHSHYTQKSLSLSPGK | |
| 349 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKCK | with M198L and N204S |
| | VSNKALPAPIEKTISKAKGQPREPQVYTLPPSRDELTKNQVSLTCLVK | mutations |
| | GFYPSDIAVLWESYGTEWASYKTTPPVLDSDGSFFLYSKLTVTKEEW | |
| | QQGFVFSCSVLHEALHSHYTQKSLSLSPGK | |
| 350 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKCK | with knob and M198L and |
| | VSNKALPAPIEKTISKAKGQPREPQVYTLPPSRDELTKNQVSLWCLVK | N204S mutations |
| | GFYPSDIAVLWESYGTEWASYKTTPPVLDSDGSFFLYSKLTVTKEEW | |
| | QQGFVFSCSVLHEALHSHYTQKSLSLSPGK | |
| 351 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKCK | with knob, LALA, and |
| | VSNKALPAPIEKTISKAKGQPREPQVYTLPPSRDELTKNQVSLWCLVK | M198L and N204S |
| | GFYPSDIAVLWESYGTEWASYKTTPPVLDSDGSFFLYSKLTVTKEEW | mutations |
| | QQGFVFSCSVLHEALHSHYTQKSLSLSPGK | |
| 352 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKCK | with knob, LALAPG, and |
| | VSNKALGAPIEKTISKAKGQPREPQVYTLPPSRDELTKNQVSLWCLV | M198L and N204S |
| | KGFYPSDIAVLWESYGTEWASYKTTPPVLDSDGSFFLYSKLTVTKEE | mutations |
| | WQQGFVFSCSVLHEALHSHYTQKSLSLSPGK | |
| 353 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKCK | with hole and M198L and |
| | VSNKALPAPIEKTISKAKGQPREPQVYTLPPSRDELTKNQVSLSCAVK | N204S mutations |
| | GFYPSDIAVLWESYGTEWASYKTTPPVLDSDGSFFLVSKLTVTKEEW | |
| | QQGFVFSCSVLHEALHSHYTQKSLSLSPGK | |
| 354 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKCK | with hole, LALA, and |
| | VSNKALPAPIEKTISKAKGQPREPQVYTLPPSRDELTKNQVSLSCAVK | M198L and N204S |
| | GFYPSDIAVLWESYGTEWASYKTTPPVLDSDGSFFLVSKLTVTKEEW | mutations |
| | QQGFVFSCSVLHEALHSHYTQKSLSLSPGK | |
| 355 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKCK | with hole, LALAPG, and |
| | VSNKALGAPIEKTISKAKGQPREPQVYTLPPSRDELTKNQVSLSCAVK | M198L and N204S |
| | GFYPSDIAVLWESYGTEWASYKTTPPVLDSDGSFFLVSKLTVTKEEW | mutations |
| | QQGFVFSCSVLHEALHSHYTQKSLSLSPGK | |
| 356 | APELLGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKCK | M198L and N204S |
| | VSNKALPAPIEKTISKAKGQPREPQVYTLPPSRDELTKNQVSLTCLVK | mutations |
| | GFYPSDIAVEWESYGTEWSNYKTTPPVLDSDGSFFLYSKLTVTKEEW | |
| | QQGFVFSCSVLHEALHSHYTQKSLSLSPGK | |
| 357 | APELLGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKCK | knob and M198L and |
| | VSNKALPAPIEKTISKAKGQPREPQVYTLPPSRDELTKNQVSLWCLVK | N204S mutations |
| | GFYPSDIAVEWESYGTEWSNYKTTPPVLDSDGSFFLYSKLTVTKEEW | |
| | QQGFVFSCSVLHEALHSHYTQKSLSLSPGK | |
| 358 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKCK | knob, LALA, and M198L |
| | VSNKALPAPIEKTISKAKGQPREPQVYTLPPSRDELTKNQVSLWCLVK | and N204S mutations |
| | GFYPSDIAVEWESYGTEWSNYKTTPPVLDSDGSFFLYSKLTVTKEEW | |
| | QQGFVFSCSVLHEALHSHYTQKSLSLSPGK | |
| 359 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKCK | knob, LALAPG, and |
| | VSNKALGAPIEKTISKAKGQPREPQVYTLPPSRDELTKNQVSLWCLV | M198L and N204S |
| | KGFYPSDIAVEWESYGTEWSNYKTTPPVLDSDGSFFLYSKLTVTKEE | mutations |
| | WQQGFVFSCSVLHEALHSHYTQKSLSLSPGK | |
| 360 | APELLGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKCK | hole and M198L and |
| | VSNKALPAPIEKTISKAKGQPREPQVYTLPPSRDELTKNQVSLSCAVK | N204S mutations |
| | GFYPSDIAVEWESYGTEWSNYKTTPPVLDSDGSFFLVSKLTVTKEEW | |
| | QQGFVFSCSVLHEALHSHYTQKSLSLSPGK | |
| 361 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKCK | hole, LALA, and M198L |
| | VSNKALPAPIEKTISKAKGQPREPQVYTLPPSRDELTKNQVSLSCAVK | and N204S mutations |
| | GFYPSDIAVEWESYGTEWSNYKTTPPVLDSDGSFFLVSKLTVTKEEW | |
| | QQGFVFSCSVLHEALHSHYTQKSLSLSPGK | |
| 362 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKCK | hole, LALAPG, and |
| | VSNKALGAPIEKTISKAKGQPREPQVYTLPPSRDELTKNQVSLSCAVK | M198L and N204S |
| | GFYPSDIAVEWESYGTEWSNYKTTPPVLDSDGSFFLVSKLTVTKEEW | mutations |
| | QQGFVFSCSVLHEALHSHYTQKSLSLSPGK | |
| 363 | APELLGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKCK | M198L and N204S |
| | VSNKALPAPIEKTISKAKGQPREPQVYTLPPSRDELTKNQVSLTCLVK | mutations |
| | GFYPSDIAVWWESYGTEWSSYKTTPPVLDSDGSFFLYSKLTVTKEEW | |
| | QQGFVFSCSVLHEALHSHYTQKSLSLSPGK | |
| 364 | APELLGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKCK | knob and M198L and |
| | VSNKALPAPIEKTISKAKGQPREPQVYTLPPSRDELTKNQVSLWCLVK | N204S mutations |
| | GFYPSDIAVWWESYGTEWSSYKTTPPVLDSDGSFFLYSKLTVTKEEW | |
| | QQGFVFSCSVLHEALHSHYTQKSLSLSPGK | |
| 365 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKCK | knob, LALA, and M198L |
| | VSNKALPAPIEKTISKAKGQPREPQVYTLPPSRDELTKNQVSLWCLVK | and N204S mutations |
| | GFYPSDIAVWWESYGTEWSSYKTTPPVLDSDGSFFLYSKLTVTKEEW | |
| | QQGFVFSCSVLHEALHSHYTQKSLSLSPGK | |
| 366 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKCK | knob, LALAPG, and |
| | VSNKALGAPIEKTISKAKGQPREPQVYTLPPSRDELTKNQVSLWCLV | M198L and N204S |
| | KGFYPSDIAVWWESYGTEWSSYKTTPPVLDSDGSFFLYSKLTVTKEE | mutations |
| | WQQGFVFSCSVLHEALHSHYTQKSLSLSPGK | |
| 367 | APELLGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKCK | hole and M198L and |
| | VSNKALPAPIEKTISKAKGQPREPQVYTLPPSRDELTKNQVSLSCAVK | N204S mutations |
| | GFYPSDIAVWWESYGTEWSSYKTTPPVLDSDGSFFLVSKLTVTKEEW | |
| | QQGFVFSCSVLHEALHSHYTQKSLSLSPGK | |
| 368 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKCK | hole, LALA, and M198L |
| | VSNKALPAPIEKTISKAKGQPREPQVYTLPPSRDELTKNQVSLSCAVK | and N204S mutations |
| | GFYPSDIAVWWESYGTEWSSYKTTPPVLDSDGSFFLVSKLTVTKEEW | |
| | QQGFVFSCSVLHEALHSHYTQKSLSLSPGK | |
| 369 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKCK | hole, LALAPG, and |
| | VSNKALGAPIEKTISKAKGQPREPQVYTLPPSRDELTKNQVSLSCAVK | M198L and N204S |
| | GFYPSDIAVWWESYGTEWSSYKTTPPVLDSDGSFFLVSKLTVTKEEW | mutations |
| | QQGFVFSCSVLHEALHSHYTQKSLSLSPGK | |
| 370 | APELLGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | with M198L and N204S |
| | VSNKALPAPIEKTISKAKGQPREPQVYTLPPSRDELTKNQVSLTCLVK | mutations |
| | GFYPSDIAVEWESFGTEWSSYKTTPPVLDSDGSFFLYSKLTVSKEEW | |
| | QQGFVFSCSVLHEALHSHYTQKSLSLSPGK | |
| 371 | APELLGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | with knob and M198L and |
| | VSNKALPAPIEKTISKAKGQPREPQVYTLPPSRDELTKNQVSLWCLVK | N204S mutations |
| | GFYPSDIAVEWESFGTEWSSYKTTPPVLDSDGSFFLYSKLTVSKEEW | |
| | QQGFVFSCSVLHEALHSHYTQKSLSLSPGK | |
| 372 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | with knob, LALA, and |
| | VSNKALPAPIEKTISKAKGQPREPQVYTLPPSRDELTKNQVSLWCLVK | M198L and N204S |
| | GFYPSDIAVEWESFGTEWSSYKTTPPVLDSDGSFFLYSKLTVSKEEW | mutations |
| | QQGFVFSCSVLHEALHSHYTQKSLSLSPGK | |
| 373 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | with knob, LALAPG, and |
| | VSNKALGAPIEKTISKAKGQPREPQVYTLPPSRDELTKNQVSLWCLV | M198L and N204S |
| | KGFYPSDIAVEWESFGtEWSSYKTTPPVLDSDGSFFLYSKLTVSKEE | mutations |
| | WQQGFVFSCSVLHEALHSHYTQKSLSLSPGK | |
| 374 | APELLGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | with hole and M198L and |
| | VSNKALPAPIEKTISKAKGQPREPQVYTLPPSRDELTKNQVSLSCAVK | N204S mutations |
| | GFYPSDIAVEWESFGTEWSSYKTTPPVLDSDGSFFLVSKLTVSKEEW | |
| | QQGFVFSCSVLHEALHSHYTQKSLSLSPGK | |
| 375 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | with hole, LALA, and |
| | VSNKALPAPIEKTISKAKGQPREPQVYTLPPSRDELTKNQVSLSCAVK | M198L and N204S |
| | GFYPSDIAVEWESFGTEWSSYKTTPPVLDSDGSFFLVSKLTVSKEEW | mutations |
| | QQGFVFSCSVLHEALHSHYTQKSLSLSPGK | |
| 376 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKCK | with hole, LALAPG, and |
| | VSNKALGAPIEKTISKAKGQPREPQVYTLPPSRDELTKNQVSLSCAVK | M198L and N204S |
| | GFYPSDIAVEWESFGTEWSSYKTTPPVLDSDGSFFLVSKLTVSKEEW | mutations |
| | QQGFVFSCSVLHEALHSHYTQKSLSLSPGK | |
| 377 | APELLGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | with M198L and N204S |
| | VSNKALPAPIEKTISKAKGQPREPQVYTLPPSRDELTKNQVSLTCLVK | mutations |
| | GFYPSDIAVEWESYGTEWANYKTTPPVLDSDGSFFLYSKLTVSKSEW | |
| | QQGFVFSCSVLHEALHSHYTQKSLSLSPGK | |
| 378 | APELLGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | with knob and M198L and |
| | VSNKALPAPIEKTISKAKGQPREPQVYTLPPSRDELTKNQVSLWCLVK | N204S mutations |
| | GFYPSDIAVEWESYGTEWANYKTTPPVLDSDGSFFLYSKLTVSKSEW | |
| | QQGFVFSCSVLHEALHSHYTQKSLSLSPGK | |
| 379 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | with knob, LALA, and |
| | VSNKALPAPIEKTISKAKGQPREPQVYTLPPSRDELTKNQVSLWCLVK | M198L and N204S |
| | GFYPSDIAVEWESYGTEWANYKTTPPVLDSDGSFFLYSKLTVSKSEW | mutations |
| | QQGFVFSCSVLHEALHSHYTQKSLSLSPGK | |
| 380 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | with knob, LALAPG, and |
| | VSNKALGAPIEKTISKAKGQPREPQVYTLPPSRDELTKNQVSLWCLV | M198L and N204S |
| | KGFYPSDIAVEWESYGTEWANYKTTPPVLDSDGSFFLYSKLTVSKSE | mutations |
| | WQQGFVFSCSVLHEALHSHYTQKSLSLSPGK | |
| 381 | APELLGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | with hole and M198L and |
| | VSNKALPAPIEKTISKAKGQPREPQVYTLPPSRDELTKNQVSLSCAVK | N204S mutations |
| | GFYPSDIAVEWESYGTEWANYKTTPPVLDSDGSFFLVSKLTVSKSEW | |
| | QQGFVFSCSVLHEALHSHYTQKSLSLSPGK | |
| 382 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | with hole, LALA, and |
| | VSNKALPAPIEKTISKAKGQPREPQVYTLPPSRDELTKNQVSLSCAVK | M198L and N204S |
| | GFYPSDIAVEWESYGTEWANYKTTPPVLDSDGSFFLVSKLTVSKSEW | mutations |
| | QQGFVFSCSVLHEALHSHYTQKSLSLSPGK | |
| 383 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKCK | with hole, LALAPG, and |
| | VSNKALGAPIEKTISKAKGQPREPQVYTLPPSRDELTKNQVSLSCAVK | M198L and N204S |
| | GFYPSDIAVEWESYGTEWANYKTTPPVLDSDGSFFLVSKLTVSKSEW | mutations |
| | QQGFVFSCSVLHEALHSHYTQKSLSLSPGK | |
| 384 | APELLGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKCK | with M198L and N204S |
| | VSNKALPAPIEKTISKAKGQPREPQVYTLPPSRDELTKNQVSLTCLVK | mutations |
| | GFYPSDIAVEWESFGTEWSNYKTTPPVLDSDGSFFLYSKLTVSKEEW | |
| | QQGFVFSCSVLHEALHSHYTQKSLSLSPGK | |
| 385 | APELLGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKCK | with knob and M198L and |
| | VSNKALPAPIEKTISKAKGQPREPQVYTLPPSRDELTKNQVSLWCLVK | N204S mutations |
| | GFYPSDIAVEWESFGTEWSNYKTTPPVLDSDGSFFLYSKLTVSKEEW | |
| | QQGFVFSCSVLHEALHSHYTQKSLSLSPGK | |
| 386 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKCK | with knob, LALA, and |
| | VSNKALPAPIEKTISKAKGQPREPQVYTLPPSRDELTKNQVSLWCLVK | M198L and N204S |
| | GFYPSDIAVEWESFGTEWSNYKTTPPVLDSDGSFFLYSKLTVSKEEW | mutations |
| | QQGFVFSCSVLHEALHSHYTQKSLSLSPGK | |
| 387 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKCK | with knob, LALAPG, and |
| | VSNKALGAPIEKTISKAKGQPREPQVYTLPPSRDELTKNQVSLWCLV | M198L and N204S |
| | KGFYPSDIAVEWESFGTqEWSNYKTTPPVLDSDGSFFLYSKLTVSKEE | mutations |
| | WQQGFVFSCSVLHEALHSHYTQKSLSLSPGK | |
| 388 | APELLGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKCK | with hole and M198L and |
| | VSNKALPAPIEKTISKAKGQPREPQVYTLPPSRDELTKNQVSLSCAVK | N204S mutations |
| | GFYPSDIAVEWESFGTEWSNYKTTPPVLDSDGSFFLVSKLTVSKEEW | |
| | QQGFVFSCSVLHEALHSHYTQKSLSLSPGK | |
| 389 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKCK | with hole, LALA, and |
| | VSNKALPAPIEKTISKAKGQPREPQVYTLPPSRDELTKNQVSLSCAVK | M198L and N204S |
| | GFYPSDIAVEWESFGTEWSNYKTTPPVLDSDGSFFLVSKLTVSKEEW | mutations |
| | QQGFVFSCSVLHEALHSHYTQKSLSLSPGK | |
| 390 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKCK | with hole, LALAPG, and |
| | VSNKALGAPIEKTISKAKGQPREPQVYTLPPSRDELTKNQVSLSCAVK | M198L and N204S |
| | GFYPSDIAVEWESFGTEWSNYKTTPPVLDSDGSFFLVSKLTVSKEEW | mutations |
| | QQGFVFSCSVLHEALHSHYTQKSLSLSPGK | |
| 391 | APELLGGPSVFLFPPKPKDTLMISRTPEVTCVVVDVSHEDPEVKFNW | Fc sequence with knob |
| | YVDGVEVHNAKTKPREEQYNSTYRVVSVLTVLHQDWLNGKEYKCK | mutation |
| | VSNKALPAPIEKTISKAKGQPREPQVYTLPPSRDELTKNQVSLWCLVK | |
| | GFYPSDIAVEWESNGQPENNYKTTPPVLDSDGSFFLYSKLTVDKSRW | |
| | QQGNVFSCSVMHEALHNHYTQKSLSLSPGK | |
| 392 | APEAAGGPSVFLFPPKPKDTLMISRTPEVTCVVVDVSHEDPEVKFNW | Fc sequence with knob and |
| | YVDGVEVHNAKTKPREEQYNSTYRVVSVLTVLHQDWLNGKEYKCK | LALA mutations |
| | VSNKALPAPIEKTISKAKGQPREPQVYTLPPSRDELTKNQVSLWCLVK | |
| | GFYPSDIAVEWESNGQPENNYKTTPPVLDSDGSFFLYSKLTVDKSRW | |
| | QQGNVFSCSVMHEALHNHYTQKSLSLSPGK | |
| 393 | APEAAGGPSVFLFPPKPKDTLMISRTPEVTCVVVDVSHEDPEVKFNW | Fc sequence with knob and |
| | YVDGVEVHNAKTKPREEQYNSTYRVVSVLTVLHQDWLNGKEYKCK | LALAPG mutations |
| | VSNKALGAPIEKTISKAKGQPREPQVYTLPPSRDELTKNQVSLWCLV | |
| | KGFYPSDIAVEWESNGQPENNYKTTPPVLDSDGSFFLYSKLTVDKSR | |
| | WQQGNVFSCSVMHEALHNHYTQKSLSLSPGK | |
| 394 | APELLGGPSVFLFPPKPKDTLYITREPEVTCVVVDVSHEDPEVKFNWY | Fc sequence with knob and |
| | VDGVEVHNAKTKPREEQYNSTYRVVSVLTVLHQDWLNGKEYKCKV | YTE mutations |
| | SNKALPAPIEKTISKAKGQPREPQVYTLPPSRDELTKNQVSLWCLVKG | |
| | FYPSDIAVEWESNGQPENNYKTTPPVLDSDGSFFLYSKLTVDKSRWQ | |
| | QGNVFSCSVMHEALHNHYTQKSLSLSPGK | |
| 395 | APEAAGGPSVFLFPPKPKDTLYITREPEVTCVVVDVSHEDPEVKFNW | Fc sequence with knob, |
| | YVDGVEVHNAKTKPREEQYNSTYRVVSVLTVLHQDWLNGKEYKCK | LALA, and YTE |
| | VSNKALPAPIEKTISKAKGQPREPQVYTLPPSRDELTKNQVSLWCLVK | mutations |
| | GFYPSDIAVEWESNGQPENNYKTTPPVLDSDGSFFLYSKLTVDKSRW | |
| | QQGNVFSCSVMHEALHNHYTQKSLSLSPGK | |
| 396 | APEAAGGPSVFLFPPKPKDTLYITREPEVTCVVVDVSHEDPEVKFNW | Fc sequence with knob, |
| | YVDGVEVHNAKTKPREEQYNSTYRVVSVLTVLHQDWLNGKEYKCK | LALAPG, and YTE |
| | VSNKALGAPIEKTISKAKGQPREPQVYTLPPSRDELTKNQVSLWCLV | mutations |
| | KGFYPSDIAVEWESNGQPENNYKTTPPVLDSDGSFFLYSKLTVDKSR | |
| | WQQGNVFSCSVMHEALHNHYTQKSLSLSPGK | |
| 397 | APELLGGPSVFLFPPKPKDTLMISRTPEVTCVVVDVSHEDPEVKFNW | Fc sequence with hole |
| | YVDGVEVHNAKTKPREEQYNSTYRVVSVLTVLHQDWLNGKEYKCK | mutations |
| | VSNKALPAPIEKTISKAKGQPREPQVYTLPPSRDELTKNQVSLSCAVK | |
| | GFYPSDIAVEWESNGQPENNYKTTPPVLDSDGSFFLVSKLTVDKSRW | |
| | QQGNVFSCSVMHEALHNHYTQKSLSLSPGK | |
| 398 | APEAAGGPSVFLFPPKPKDTLMISRTPEVTCVVVDVSHEDPEVKFNW | Fc sequence with hole and |
| | YVDGVEVHNAKTKPREEQYNSTYRVVSVLTVLHQDWLNGKEYKCK | LALA mutations |
| | VSNKALPAPIEKTISKAKGQPREPQVYTLPPSRDELTKNQVSLSCAVK | |
| | GFYPSDIAVEWESNGQPENNYKTTPPVLDSDGSFFLVSKLTVDKSRW | |
| | QQGNVFSCSVMHEALHNHYTQKSLSLSPGK | |
| 399 | APEAAGGPSVFLFPPKPKDTLMISRTPEVTCVVVDVSHEDPEVKFNW | Fc sequence with hole and |
| | YVDGVEVHNAKTKPREEQYNSTYRVVSVLTVLHQDWLNGKEYKCK | LALAPG mutations |
| | VSNKALGAPIEKTISKAKGQPREPQVYTLPPSRDELTKNQVSLSCAVK | |
| | GFYPSDIAVEWESNGQPENNYKTTPPVLDSDGSFFLVSKLTVDKSRW | |
| | QQGNVFSCSVMHEALHNHYTQKSLSLSPGK | |
| 400 | APELLGGPSVFLFPPKPKDTLYITREPEVTCVVVDVSHEDPEVKFNWY | Fc sequence with hole and |
| | VDGVEVHNAKTKPREEQYNSTYRVVSVLTVLHQDWLNGKEYKCKV | YTE mutations |
| | SNKALPAPIEKTISKAKGQPREPQVYTLPPSRDELTKNQVSLSCAVKG | |
| | FYPSDIAVEWESNGQPENNYKTTPPVLDSDGSFFLVSKLTVDKSRWQ | |
| | QGNVFSCSVMHEALHNHYTQKSLSLSPGK | |
| 401 | APEAAGGPSVFLFPPKPKDTLYITREPEVTCVVVDVSHEDPEVKFNW | Fc sequence with hole, |
| | YVDGVEVHNAKTKPREEQYNSTYRVVSVLTVLHQDWLNGKEYKCK | LALA, and YTE |
| | VSNKALPAPIEKTISKAKGQPREPQVYTLPPSRDELTKNQVSLSCAVK | mutations |
| | GFYPSDIAVEWESNGQPENNYKTTPPVLDSDGSFFLVSKLTVDKSRW | |
| | QQGNVFSCSVMHEALHNHYTQKSLSLSPGK | |
| 402 | APEAAGGPSVFLFPPKPKDTLYITREPEVTCVVVDVSHEDPEVKFNW | Fc sequence with hole, |
| | YVDGVEVHNAKTKPREEQYNSTYRVVSVLTVLHQDWLNGKEYKCK | LALAPG, and YTE |
| | VSNKALGAPIEKTISKAKGQPREPQVYTLPPSRDELTKNQVSLSCAVK | mutations |
| | GFYPSDIAVEWESNGQPENNYKTTPPVLDSDGSFFLVSKLTVDKSRW | |
| | QQGNVFSCSVMHEALHNHYTQKSLSLSPGK | |
| 403 | APELLGGPSVFLFPPKPKDTLMISRTPEVTCVVVDVSHEDPEVKFNW | Fc sequence with M198L |
| | YVDGVEVHNAKTKPREEQYNSTYRVVSVLTVLHQDWLNGKEYKCK | and N204S mutations |
| | VSNKALPAPIEKTISKAKGQPREPQVYTLPPSRDELTKNQVSLTCLVK | |
| | GFYPSDIAVEWESNGQPENNYKTTPPVLDSDGSFFLYSKLTVDKSRW | |
| | QQGNVFSCSVLHEALHSHYTQKSLSLSPGK | |
| 404 | APELLGGPSVFLFPPKPKDTLMISRTPEVTCVVVDVSHEDPEVKFNW | Fc sequence with knob and |
| | YVDGVEVHNAKTKPREEQYNSTYRVVSVLTVLHQDWLNGKEYKCK | M198L and N204S |
| | VSNKALPAPIEKTISKAKGQPREPQVYTLPPSRDELTKNQVSLWCLVK | mutations |
| | GFYPSDIAVEWESNGQPENNYKTTPPVLDSDGSFFLYSKLTVDKSRW | |
| | QQGNVFSCSVLHEALHSHYTQKSLSLSPGK | |
| 405 | APEAAGGPSVFLFPPKPKDTLMISRTPEVTCVVVDVSHEDPEVKFNW | Fc sequence with knob, |
| | YVDGVEVHNAKTKPREEQYNSTYRVVSVLTVLHQDWLNGKEYKCK | LALA, and M198L and |
| | VSNKALPAPIEKTISKAKGQPREPQVYTLPPSRDELTKNQVSLWCLVK | N204S mutations |
| | GFYPSDIAVEWESNGQPENNYKTTPPVLDSDGSFFLYSKLTVDKSRW | |
| | QQGNVFSCSVLHEALHSHYTQKSLSLSPGK | |
| 406 | APEAAGGPSVFLFPPKPKDTLMISRTPEVTCVVVDVSHEDPEVKFNW | Fc sequence with knob, |
| | YVDGVEVHNAKTKPREEQYNSTYRVVSVLTVLHQDWLNGKEYKCK | LALAPG, and M198L and |
| | VSNKALGAPIEKTISKAKGQPREPQVYTLPPSRDELTKNQVSLWCLV | N204S mutations |
| | KGFYPSDIAVEWESNGQPENNYKTTPPVLDSDGSFFLYSKLTVDKSR | |
| | WQQGNVFSCSVLHEALHSHYTQKSLSLSPGK | |
| 407 | APELLGGPSVFLFPPKPKDTLMISRTPEVTCVVVDVSHEDPEVKFNW | Fc sequence with hole and |
| | YVDGVEVHNAKTKPREEQYNSTYRVVSVLTVLHQDWLNGKEYKCK | M198L and N204S |
| | VSNKALPAPIEKTISKAKGQPREPQVYTLPPSRDELTKNQVSLSCAVK | mutations |
| | GFYPSDIAVEWESNGQPENNYKTTPPVLDSDGSFFLVSKLTVDKSRW | |
| | QQGNVFSCSVLHEALHSHYTQKSLSLSPGK | |
| 408 | APEAAGGPSVFLFPPKPKDTLMISRTPEVTCVVVDVSHEDPEVKFNW | Fc sequence with hole, |
| | YVDGVEVHNAKTKPREEQYNSTYRVVSVLTVLHQDWLNGKEYKCK | LALA, and M198L and |
| | VSNKALPAPIEKTISKAKGQPREPQVYTLPPSRDELTKNQVSLSCAVK | N204S mutations |
| | GFYPSDIAVEWESNGQPENNYKTTPPVLDSDGSFFLVSKLTVDKSRW | |
| | QQGNVFSCSVLHEALHSHYTQKSLSLSPGK | |
| 409 | APEAAGGPSVFLFPPKPKDTLMISRTPEVTCVVVDVSHEDPEVKFNW | Fc sequence with hole, |
| | YVDGVEVHNAKTKPREEQYNSTYRVVSVLTVLHQDWLNGKEYKCK | LALAPG, and M198L and |
| | VSNKALGAPIEKTISKAKGQPREPQVYTLPPSRDELTKNQVSLSCAVK | N204S mutations |
| | GFYPSDIAVEWESNGQPENNYKTTPPVLDSDGSFFLVSKLTVDKSRW | |
| | QQGNVFSCSVLHEALHSHYTQKSLSLSPGK | |
| 410 | QVQLQQPGAELVKPGASVKMSCKASGYTFTSYNMHWVKQTPGRGL | Heavy chain for anti- |
| | EWIGAIYPGNGDTSYNQKFKGKATLTADKSSSTAYMQLSSLTSEDSA | hCD20-3C.35.23 with |
| | VYYCARSTYYGGDWYFNVWGAGTTVTVSAASTKGPSVFPLAPSSKS | knob mutations |
| | TSGGTAALGCLVKDYFPEPVTVSWNSGALTSGVHTFPAVLQSSGLYS | |
| | LSSVVTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLWCLVK | |
| | GFYPSDIAVEWESYGTEWSNYKTTPPVLDSDGSFFLYSKLTVTKEEW | |
| | QQGFVFSCSVMHEALHNHYTQKSLSLSPGK | |
| 411 | QVQLQQPGAELVKPGASVKMSCKASGYTFTSYNMHWVKQTPGRGL | Heavy chain for anti- |
| | EWIGAIYPGNGDTSYNQKFKGKATLTADKSSSTAYMQLSSLTSEDSA | hCD20-3C.35.23 with |
| | VYYCARSTYYGGDWYFNVWGAGTTVTVSAASTKGPSVFPLAPSSKS | knob and LALA mutations |
| | TSGGTAALGCLVKDYFPEPVTVSWNSGALTSGVHTFPAVLQSSGLYS | |
| | LSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFN | |
| | WYVDGVEVHNAKTKPREEQYNSTYRVVSVLTVLHQDWLNGKEYK | |
| | CKVSNKALPAPIEKTISKAKGQPREPQVYTLPPSRDELTKNQVSLWCL | |
| | VKGFYPSDIAVEWESYGTEWSNYKTTPPVLDSDGSFFLYSKLTVTKE | |
| | EWQQGFVFSCSVMHEALHNHYTQKSLSLSPGK | |
| 412 | QVQLQQPGAELVRPGTSVKLSCKASGYTFTSYWMHWIKQRPGQGLE | Heavy chain for anti- |
| | WIGVIDPSDNYTKYNQKFKGKATLTVDTSSSTAYMQLSSLTSEDSAV | mCD20-3C.35.23 with |
| | YFCAREGYYGSSPWFAYWGQGTLVTVSSASTKGPSVFPLAPSSKSTS | knob mutations |
| | GGTAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPPCP | |
| | APELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLWCLVK | |
| | GFYPSDIAVEWESYGTEWSNYKTTPPVLDSDGSFFLYSKLTVTKEEW | |
| | QQGFVFSCSVMHEALHNHYTQKSLSLSPGK | |
| 413 | QVQLQQPGAELVRPGTSVKLSCKASGYTFTSYWMHWIKQRPGQGLE | Heavy chain for anti- |
| | WIGVIDPSDNYTKYNQKFKGKATLTVDTSSSTAYMQLSSLTSEDSAV | mCD20-3C.35.23 with |
| | YFCAREGYYGSSPWFAYWGQGTLVTVSSASTKGPSVFPLAPSSKSTS | knob and LALA mutations |
| | GGTAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPPCP | |
| | APEAAGGPSVFLFPPKPKDTLMISRTPEVTCVVVDVSHEDPEVKFNW | |
| | YVDGVEVHNAKTKPREEQYNSTYRVVSVLTVLHQDWLNGKEYKCK | |
| | VSNKALPAPIEKTISKAKGQPREPQVYTLPPSRDELTKNQVSLWCLVK | |
| | GFYPSDIAVEWESYGTEWSNYKTTPPVLDSDGSFFLYSKLTVTKEEW | |
| | QQGFVFSCSVMHEALHNHYTQKSLSLSPGK | |
| 414 | YxtEWSS | Consensus motif for |
| | | CH3C.35 |
| 415 | TxxExxxxF | Consensus motif for |
| | | CH3C.35 |
| 416 | QVQLVESGGGVVQPGRSLRLSCAASGFAFSSYGMHWVRQAPGKGLE | Heavy chain for anti-Aβ- |
| | WVAVIWFDGTKKYYTDSVKGRFTISRDNSKNTLYLQMNTLRAEDTA | Fc polypeptide |
| | VYYCARDRGIGARRGPYYMDVWGKGTTVTVSSASTKGPSVFPLAPS | |
| | SKSTSGGTAALGCLVKDYFPEPVTVSWNSGALTSGVHTFPAVLQSSG | |
| | LYSLSSVVTVPSSSLGTQTYICNVNHKPSNTKVDKKVEPKSCDKTHT | |
| | CPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKE | |
| | YKCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTVD | |
| | KSRWQQGNVFSCSVMHEALHNHYTQKSLSLSPGK | |
| 417 | QVQLVESGGGVVQPGRSLRLSCAASGFAFSSYGMHWVRQAPGKGLE | Heavy chain for anti-Aβ- |
| | WVAVIWFDGTKKYYTDSVKGRFTISRDNSKNTLYLQMNTLRAEDTA | 3C.35.23.4 with knob and |
| | VYYCARDRGIGARRGPYYMDVWGKGTTVTVSSASTKGPSVFPLAPS | LALA mutations |
| | SKSTSGGTAALGCLVKDYFPEPVTVSWNSGALTSGVHTFPAVLQSSG | |
| | LYSLSSVVTVPSSSLGTQTYICNVNHKPSNTKVDKKVEPKSCDKTHT | |
| | CPPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNGKE | |
| | YKCKVSNKALPAPIEKTISKAKGQPREPQVYTLPPSRDELTKNQVSL | |
| | WCLVKGFYPSDIAVEWESYGTEWSNYKTTPPVLDSDGSFFLYSKLTV | |
| | SKEEWQQGFVFSCSVMHEALHNHYTQKSLSLSPGK | |
| 418 | QVQLVESGGGVVQPGRSLRLSCAASGFAFSSYGMHWVRQAPGKGLE | Heavy chain for anti-Aβ- |
| | WVAVIWFDGTKKYYTDSVKGRFTISRDNSKNTLYLQMNTLRAEDTA | Fc with hole and LALA |
| | VYYCARDRGIGARRGPYYMDVWGKGTTVTVSSASTKGPSVFPLAPS | mutations |
| | SKSTSGGTAALGCLVKDYFPEPVTVSWNSGALTSGVHTFPAVLQSSG | |
| | LYSLSSVVTVPSSSLGTQTYICNVNHKPSNTKVDKKVEPKSCDKTHT | |
| | CPPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNGKE | |
| | YKCKVSNKALPAPIEKTISKAKGQPREPQVYTLPPSRDELTKNQVSLS | |
| | CAVKGFYPSDIAVEWESNGQPENNYKTTPPVLDSDGSFFLVSKLTVD | |
| | KSRWQQGNVFSCSVMHEALHNHYTQKSLSLSPGK | |
| 419 | QVQLVESGGGVVQPGRSLRLSCAASGFAFSSYGMHWVRQAPGKGLE | Heavy chain for anti-Aβ- |
| | WVAVIWFDGTKKYYTDSVKGRFTISRDNSKNTLYLQMNTLRAEDTA | Fc with hole mutations |
| | VYYCARDRGIGARRGPYYMDVWGKGTTVTVSSASTKGPSVFPLAPS | |
| | SKSTSGGTAALGCLVKDYFPEPVTVSWNSGALTSGVHTFPAVLQSSG | |
| | LYSLSSVVTVPSSSLGTQTYICNVNHKPSNTKVDKKVEPKSCDKTHT | |
| | CPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKE | |
| | YKCKVSNKALPAPIEKTISKAKGQPREPQVYTLPPSRDELTKNQVSLS | |
| | CAVKGFYPSDIAVEWESNGQPENNYKTTPPVLDSDGSFFLVSKLTVD | |
| | KSRWQQGNVFSCSVMHEALHNHYTQKSLSLSPGK | |
| 420 | DIQMTQSPSSLSASVGDRVTITCRASQSISSYLNWYQQKPGKAPKLLI | Light chain for anti-Aβ |
| | YAASSLQSGVPSRFSGSGSGTDFTLTISSLQPEDFATYYCQQSYSTPLT | fusion |
| | FGGGTKVEIKRTVAAPSVFIFPPSDEQLKSGTASVVCLLNNFYPREAK | |
| | VQWKVDNALQSGNSQESVTEQDSKDSTYSLSSTLTLSKADYEKHKV | |
| | YACEVTHQGLSSPVTKSFNRGEC | |
| 421 | HHHHHH | 6XHis tag |

1. A modified Fc polypeptide dimer, or a dimeric fragment thereof, that:
(a) specifically binds TfR;
(b) is capable of binding an Fcγ receptor (FcγR); and
(c) does not substantially deplete reticulocytes in vivo.

2. A modified Fc polypeptide dimer, or a dimeric fragment thereof, comprising:
(a) a first Fc polypeptide that specifically binds TfR comprising (i) a TfR-binding site and (ii) one or more amino acid modifications that reduce FcγR binding when bound to TfR; and
(b) a second Fc polypeptide that does not contain a TfR-binding site or any modifications that reduce FcγR binding.

3. The modified Fc polypeptide dimer of claim 1, wherein the first Fc polypeptide comprises a modified CH3 domain comprising the TfR-binding site.

4. (canceled)

5. The modified Fc polypeptide dimer of claim 3, wherein the modified CH3 domain comprises five, six, seven, eight, or nine substitutions in a set of amino acid positions comprising 384, 386, 387, 388, 389, 390, 413, 416, and 421, according to EU numbering.

6. The modified Fc polypeptide dimer of claim 5, wherein the modified CH3 domain further comprises one, two, three, or four substitutions at positions comprising 380, 391, 392, and 415.

7. (canceled)

8. The modified Fc polypeptide dimer of claim 2, wherein the modified Fc polypeptide dimer binds to the apical domain of TfR, and/or
wherein the modified Fc polypeptide dimer binds to TfR without inhibiting binding of transferrin to TfR, and/or
wherein the modified Fc polypeptide dimer binds to an epitope that comprises amino acid 208 of TfR.

9-10. (canceled)

11. The modified Fc polypeptide dimer of claim 5, wherein the modified CH3 domain comprises Trp at position 388, and/or Trp or Phe at position 421.

12-14. (canceled)

15. The modified Fc polypeptide dimer of claim 5, wherein the modified CH3 domain comprises one, two, three, four, five, six, seven, or eight positions selected from the following: position 384 is Leu, Tyr, Met, or Val; position 386 is Leu, Thr, His, or Pro; position 387 is Val, Pro, or an acidic amino acid; position 388 is Trp; position 389 is Val, Ser, or Ala; position 413 is Glu, Ala, Ser, Leu, Thr, or Pro; position 416 is Thr or an acidic amino acid; and position 421 is Trp, Tyr, His, or Phe.

16-21. (canceled)

22. The modified Fc polypeptide dimer of claim 5, wherein:
(a) the modified CH3 domain comprises Tyr at position 384, Thr at position 386, Glu or Val and position 387, Trp at position 388, Ser at position 389, Ser or Thr at position 413, Glu at position 416, and/or Phe at position 421; or
(b) the modified CH3 domain comprises Tyr at position 384, Thr at position 386, Glu or Val and position 387, Trp at position 388, Ser at position 389, Ser or Thr at position 413, Glu at position 415, Glu at position 416, and/or Phe at position 421, or
(c) the modified CH3 domain comprises Tyr at position 384, Thr at position 386, Glu or Val and position 387, Trp at position 388, Ser at position 389, Asn at position 390, Ser or Thr at position 413, Glu at position 416, and/or Phe at position 421, or
(d) the modified CH3 domain comprises Tyr at position 384, Thr at position 386, Glu or Val and position 387, Trp at position 388, Ser at position 389, Asn at position 390, Ser or Thr at position 413, Glu at position 415, Glu at position 416, and/or Phe at position 421.

23-27. (canceled)

28. The modified Fc polypeptide dimer of claim 5, wherein the modified CH3 domain has at least 85% identity to amino acids 111-217 of any one of SEQ ID NOS:13, 24-29, 64-69, and 76-127.

29. The modified Fc polypeptide dimer of claim 28, wherein the modified CH3 domain has at least 85% identity to amino acids 111-217 of any one of SEQ ID NOS:66, 68, 94, 107-109, 119, and 268-270.

30. The modified Fc polypeptide dimer of claim 5, wherein the modified CH3 domain has at least 85% identity to amino acids 111-217 of SEQ ID NO:1 with the proviso that the percent identity does not include the set of positions 384, 386, 387, 388, 389, 390, 413, 416, and 421, according to EU numbering.

31-32. (canceled)

33. The modified Fc polypeptide dimer of claim 6, wherein the modified CH3 domain comprises 1, 2, 3, 4, 5, 6, 7, 8, 9, 10, or 11 positions selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

34-40. (canceled)

41. The modified Fc polypeptide dimer of claim 2, wherein the amino acid modifications that reduce FcγR binding when bound to TfR comprise Ala at position 234 and at position 235, according to EU numbering scheme.

42. The modified Fc polypeptide dimer of claim 2, wherein the first Fc polypeptide and/or the second Fc polypeptide comprises amino acid modifications comprising (i) a Leu at position 428 and a Ser at position 434, or (ii) a Ser or Ala at position 434, according to EU numbering scheme.

43. (canceled)

44. The modified Fc polypeptide dimer of claim 2, wherein the first Fc polypeptide and/or the second Fc polypeptide is further fused to a Fab.

45. The modified Fc polypeptide dimer of claim 2, wherein the first Fc polypeptide comprises a knob mutation T366W and the second Fc polypeptide comprises hole mutations T366S, L368A, and Y407V, according to EU numbering scheme; or
wherein the first Fc polypeptide comprises hole mutations T366S, L368A, and Y407V and the second Fc polypeptide comprises a knob mutation T366W, according to EU numbering scheme.

46-70. (canceled)

71. The modified Fc polypeptide dimer of claim 2, wherein:
(A) the modified Fc polypeptide dimer does not substantially deplete reticulocytes;
(b) an amount of reticulocytes depleted after administering the modified Fc polypeptide dimer is less than an amount of reticulocytes depleted after administering a control;
(c) an amount of reticulocytes remaining after administering the modified Fc polypeptide dimer is more than an amount of reticulocytes remaining after administering a control;
(d) the modified Fc polypeptide dimer does not substantially deplete reticulocytes in bone marrow;
(e) an amount of reticulocytes depleted in bone marrow after administering the modified Fc polypeptide dimer is less than an amount of reticulocytes depleted in bone marrow after administering a control; and/or
(f) an amount of reticulocytes remaining in bone marrow after administering the modified Fc polypeptide dimer is more than an amount of reticulocytes remaining in bone marrow after administering a control.

72-80. (canceled)

81. The modified Fc polypeptide dimer of claim 71, wherein the control is a corresponding TfR-binding Fc dimer with full effector function and/or contains no mutations that reduce FcγR binding.

82. An Fc polypeptide dimer-Fab fusion protein that is capable of being actively transported across the BBB, the Fc polypeptide dimer-Fab fusion protein comprising:
(a) an antibody variable region that is capable of binding an antigen, or antigen-binding fragment thereof; and
(b) a modified Fc polypeptide dimer comprising (i) a first Fc polypeptide that specifically binds TfR comprising a TfR-binding site and one or more amino acid modifications that reduce FcγR binding when bound to TfR, and (ii) a second Fc polypeptide that does not contain a TfR-binding site or any modifications that reduce FcγR binding.

83. The Fc polypeptide dimer-Fab fusion protein of claim 82, wherein the amino acid modifications that reduce FcγR binding when bound to TfR comprise Ala at position 234 and at position 235, according to EU numbering scheme; and/or
wherein the first Fc polypeptide and/or the second Fc polypeptide comprises amino acid modifications comprising (i) a Leu at position 428 and a Ser at position 434, or (ii) a Ser or Ala at position 434, according to EU numbering scheme.

84-87. (canceled)

88. A pharmaceutical composition comprising the modified Fc polypeptide dimer of claim 2 and a pharmaceutically acceptable carrier.

89. (canceled)

90. A method of transcytosis of a composition across an endothelium, comprising contacting the endothelium with a composition comprising a modified Fc polypeptide dimer of claim 2.

91. (canceled)

92. The method of claim 90, wherein the endothelium is the BBB.

93. An Fc polypeptide dimer-Fab fusion protein that is capable of being actively transported across the BBB, the Fc polypeptide dimer-Fab fusion protein comprising:
(a) an antibody variable region that is capable of binding an antigen, or antigen-binding fragment thereof; and
(b) a modified Fc polypeptide dimer comprising (i) a first Fc polypeptide that specifically binds TfR comprising the sequence of SEQ ID NO:214, and (ii) a second Fc polypeptide comprising the sequence of SEQ ID NO:397.

94. An Fc polypeptide dimer-Fab fusion protein that is capable of being actively transported across the BBB, the Fc polypeptide dimer-Fab fusion protein comprising:
(a) an antibody variable region that is capable of binding an antigen, or antigen-binding fragment thereof; and
(b) a modified Fc polypeptide dimer comprising (i) a first Fc polypeptide that specifically binds TfR comprising the sequence of SEQ ID NO:344, and (ii) a second Fc polypeptide comprising the sequence of SEQ ID NO:407.

95. An Fc polypeptide dimer-Fab fusion protein that is capable of being actively transported across the BBB, the Fc polypeptide dimer-Fab fusion protein comprising:
(a) an antibody variable region that is capable of binding an antigen, or antigen-binding fragment thereof; and
(b) a modified Fc polypeptide dimer comprising (i) a first Fc polypeptide that specifically binds TfR comprising the sequence of SEQ ID NO:202, and (ii) a second Fc polypeptide comprising the sequence of SEQ ID NO:397.

96. An Fc polypeptide dimer-Fab fusion protein that is capable of being actively transported across the BBB, the Fc polypeptide dimer-Fab fusion protein comprising:
(a) an antibody variable region that is capable of binding an antigen, or antigen-binding fragment thereof; and
(b) a modified Fc polypeptide dimer comprising (i) a first Fc polypeptide that specifically binds TfR comprising the sequence of SEQ ID NO:337, and (ii) a second Fc polypeptide comprising the sequence of SEQ ID NO:407.
